(19) United States
(12) Patent Application Publication
(10) Pub. No.: US 2023/0270540 A1
(43) Pub. Date: Aug. 31, 2023

(54) PROSTHETIC HEART VALVES WITH HERMETIC LAYERS OR VALVULAR STRUCTURES TO REDUCE THROMBOSIS RISK

(71) Applicant: EDWARDS LIFESCIENCES CORPORATION, Irvine, CA (US)

(72) Inventors: Evan T. Schwartz, Huntington Beach, CA (US); Darshin S. Patel, San Juan Capistrano, CA (US); Jocelyn Chau, Buena Park, CA (US); Hannah Reed Bettencourt, Huntington Beach, CA (US)

(21) Appl. No.: 18/314,759

(22) Filed: May 9, 2023

Related U.S. Application Data
(63) Continuation of application No. PCT/US2021/058608, filed on Nov. 9, 2021.
(60) Provisional application No. 63/240,766, filed on Sep. 3, 2021.
(60) Provisional application No. 63/112,080, filed on Nov. 10, 2020.

(51) Int. Cl. A61F 2/24

(52) U.S. Cl. CPC A61F 2/2412; A61F 2/2409; A61F 2220/0075

(57) ABSTRACT

A prosthetic heart valve can have one or more hermetic layers. The inner skirt and/or the outer skirt can comprise one or more hermetic layers, or the entire valve frame can be encapsulated within one or more hermetic layers. Each hermetic layer can be substantially nonporous or can have pores therein that are sized to discourage cellular ingrowth. The hermetic layer can prevent ingrowth of surrounding native tissue, thereby reducing pannus formation on the prosthetic leaflets. Alternatively or additionally, shapes of the leaflets of the valvular structure of the prosthetic valve and/or the coupling of the leaflets to the valve frame can be selected to avoid the incidence of stasis when implanted at a relatively low pressure gradient hemodynamic location. Such a prosthetic heart valve may reduce the risk of thrombosis.

### CROSS-REFERENCE TO RELATED APPLICATIONS

This application is a continuation of PCT Application No. PCT/US2021/058608, filed Nov. 9, 2021, which application claims the benefit of U.S. Provisional Application No. 63/112,080 filed Nov. 10, 2020, and U.S. Provisional No. 63/240,766 filed Sep. 3, 2021, all of which applications are incorporated herein in their entireties by this specific reference.

### FIELD

The present disclosure relates to prosthetic heart valves, in particular, hermetic layers and/or valvular structures thereof that can reduce the risk of thrombosis from the implanted prosthetic heart valves.

### BACKGROUND

The human heart can suffer from various valvular diseases, which can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped configuration on the end of a delivery device and advanced through the patient's vasculature until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery device so that the prosthetic valve can self-expand to its functional size.

Such expandable, transcatheter heart valves have an annular metal frame, a valvular structure having a plurality of leaflets supported within the frame, an inner skirt coupled to an interior of the metal frame, and an outer skirt coupled to an exterior of the metal frame. The inner and outer skirts are often constructed of a porous material, for example, woven polyethylene terephthalate (PET). The porous nature of the skirt material (e.g., having pores greater than 30-50 μm) is designed to encourage cellular ingrowth of the surrounding native tissue or from surrounding overgrown devices. This tissue ingrowth into the skirts may integrate the implanted heart valve within the native anatomy of the patient and further reduce paravalvular leakage (PVL). However, such ingrowth may propagate from the porous skirts onto the leaflets of the implanted prosthetic heart valve. Indeed, growths of tissue and pannus have been observed on leaflets in implanted prosthetic heart valves, which can act as substrates onto which thrombi may later deposit.

Moreover, in certain implantation locations, the prosthetic heart valve may be exposed to a relatively-low pressure hemodynamic environment (e.g., where the driving pressure that causes the implanted valve to open is less than 30 mmHg, for example, in the mitral or tricuspid positions). In conventional prosthetic heart valves, the low pressure gradient may cause abnormal motion of the leaflets as it transitions between open and closed configurations of the valvular structure. The leaflet motion abnormalities can result in stasis, which in turn can make the leaflets more susceptible to chronic thrombosis, thickening (which further restricts the motion of the leaflets), or both.

Accordingly, a need exists for prosthetic heart valves that reduce the risk of thrombosis from implantation thereof, and methods for implanting and assembling such prosthetic heart valves.

### SUMMARY

In one aspect, a prosthetic heart valve can be summarized as comprising an annular frame, a valvular structure, and an inner skirt. The annular frame can be radially collapsible and expandable between a radially-compressed configuration and a radially-expanded configuration. The annular frame can have an inflow end and an outflow end separated from the inflow end along an axial direction of the frame. The valvular structure can be supported within the annular frame. The valvular structure can comprise a plurality of leaflets. Each leaflet can have a cusp edge portion and tabs on opposite sides with respect to a centerline of the leaflet. The cusp edge portion can be curved along at least a portion thereof to form an apex at the centerline of the leaflet. The valvular structure can be coupled to the frame via a plurality of commissure assemblies formed by paired tabs of adjacent leaflets. The inner skirt can be disposed on a radially-inner circumferential surface of the annular frame and coupled thereto. The inner skirt can comprise a hermetic layer constructed such that, when the prosthetic heart valve is implanted in a patient, ingrowth of cells from surrounding native tissue of the patient into the hermetic layer is prevented. The inner skirt can be disposed between the annular frame and the cusp edge portion of each leaflet along a radial direction of the annular frame. The inner skirt can extend along the axial direction of the frame from at least the apices of the cusp edge portions of the leaflets to at least the plurality of commissure assemblies.

In another aspect, a prosthetic heart valve can be summarized as comprising an annular frame and a valvular structure. The annular frame can be radially collapsible and expandable between a radially-compressed configuration and a radially-expanded configuration. The annular frame can have an inflow end and an outflow end separated from the inflow end along an axial direction of the frame. The valvular structure can be supported within the annular frame and can comprise a plurality of leaflets. Each leaflet can have a cusp edge portion and tabs on opposite sides with respect to a centerline of the leaflet. The cusp edge portion can be curved along at least a portion thereof to form an apex at the centerline of the leaflet. The valvular structure can be coupled to the frame via a plurality of commissure assemblies formed by paired tabs of adjacent leaflets. The annular frame can be encapsulated by a hermetic layer constructed such that, when the prosthetic heart valve is implanted in a patient, ingrowth of cells from surrounding native tissue of the patient into the hermetic layer is prevented.

In another aspect, a prosthetic heart valve can comprise an annular frame, a valvular structure, and means for preventing ingrowth of cells from surrounding native tissue of a patient onto leaflets of the valvular structure. The annular frame can have an inflow end and an outflow end separated from the inflow end along an axial direction of the frame. The valvular structure can be supported within the annular frame and can comprise a plurality of leaflets. Each leaflet can have a cusp edge portion and tabs on opposite sides with respect to a centerline of the leaflet. The cusp edge portion can be curved along at least a portion thereof. The valvular structure can be coupled to the frame via a plurality of commissure assemblies formed by paired tabs of adjacent leaflets.

In another aspect, a prosthetic heart valve can be summarized as comprising a frame, a valvular structure, and means for preventing ingrowth of cells from surrounding native tissue of a patient onto leaflets of the valvular structure. The valvular structure can be coupled to the frame and can comprise a plurality of leaflets.

In another aspect, an assembly can be summarized as comprising a delivery apparatus and a prosthetic heart valve. The delivery apparatus can comprise an elongated shaft. The prosthetic heart valve can be according to any of the above noted examples. The prosthetic heart valve can be mounted on the elongated shaft in the radially-compressed configuration for delivery into a patient's body.

In another aspect, a method of implanting a prosthetic heart valve in a patient's body can be summarized as comprising inserting a distal end of a delivery apparatus into vasculature of a patient. The delivery apparatus can comprise an elongated shaft. The prosthetic heart valve can be according to any of the above noted examples and can be releasably mounted in the radially-compressed configuration on the elongated shaft of the delivery apparatus.

In another aspect, a method of assembling a prosthetic heart valve can be summarized as comprising providing an inner skirt on a radially-inner circumferential surface of an annular frame. The annular frame can be radially collapsible and expandable between a radially-compressed configuration and a radially-expanded configuration. The annular frame can have an inflow end and an outflow end separated from the inflow end along an axial direction of the frame. The inner skirt can comprise a hermetic layer. The hermetic layer can comprise a layer formed directly on the radially-inner circumferential surface of the annular frame. The hermetic layer can be constructed such that, when the prosthetic heart valve is implanted in a patient, ingrowth of cells from surrounding native tissue of the patient into the hermetic layer is prevented.

In another aspect, a method of assembling a prosthetic heart valve can be summarized as comprising coupling an inner skirt to a radially-inner circumferential surface of an annular frame. The annular frame can be radially collapsible and expandable between a radially-compressed configuration and a radially-expanded configuration. The annular frame can have an inflow end and an outflow end separated from the inflow end along an axial direction of the frame. The inner skirt can comprise a hermetic layer constructed such that, when the prosthetic heart valve is implanted in a patient, ingrowth of cells from surrounding native tissue of the patient into the hermetic layer is prevented.

In another aspect, a method of assembling a prosthetic heart valve can be summarized as comprising encapsulating an annular frame with a hermetic layer. The annular frame can be radially collapsible and expandable between a radially-compressed configuration and a radially-expanded configuration. The annular frame can have an inflow end and an outflow end separated from the inflow end along an axial direction of the frame. The hermetic layer can be constructed such that, when the prosthetic heart valve is implanted in a patient, ingrowth of cells from surrounding native tissue of the patient into the hermetic layer is prevented.

In another aspect, a leaflet for a valvular structure of a prosthetic heart valve can be summarized as comprising a first portion, a second portion, and first and second tabs. The first and second tabs can be on opposite sides of the first portion with respect to a centerline of the first portion. Each of the tabs can have a base edge and an outer edge. The outer edges of the first and second tabs can be substantially parallel to each other. The second portion can have a half-elliptical or semi-elliptical shape that defines a cusp edge. The cusp edge can extend from the base edge of the first tab to the base edge of the second tab. The cusp edge can be curved along its entire length between the base edges of the first and second tabs.

In another aspect, a prosthetic heart valve can be summarized as comprising an annular frame, a valvular structure, and an inner skirt. The annular frame can be radially collapsible and expandable between a radially-compressed configuration and a radially-expanded configuration. The annular frame can have an inflow end and an outflow end separated from the inflow end along an axial direction of the frame. The valvular structure can be supported within the annular frame and can comprise a plurality of leaflets. Each leaflet can be according to any of the above noted examples. The valvular structure can be coupled to the frame via a plurality of commissure assemblies formed by paired ones of the tabs from adjacent leaflets. The inner skirt can be disposed on a radially-inner circumferential surface of the annular frame and can be coupled thereto. A cusp edge portion of the second portion of each leaflet at the respective cusp edge can be coupled to the inner skirt. One or more sutures can couple the cusp edge portion of each leaflet to the inner skirt, and a suture line formed by the one or more sutures can follow a curvature of the cusp edge. The suture line formed by the one or more sutures can be continuous from apices of the cusp edges substantially to the commissure assemblies.

In another aspect, a prosthetic heart valve can be summarized as comprising one or more frames and a valvular structure supported within the one or more frames. The one or more frames can be radially collapsible and expandable between a radially-compressed configuration and a radially-expanded configuration. The one or more frames can define an inflow end of the prosthetic heart valve and an outflow end of the prosthetic heart valve, the outflow end being separated from the inflow end along an axial direction of the one or more frames. The valvular structure can comprise a plurality of leaflets. Each leaflet can have a first portion, a second portion, a first tab, and a second tab. The first and second tabs can be on opposite sides of the first portion with respect to a centerline of the first portion. Each of the tabs can have a base edge and an outer edge. The outer edges of the first and second tabs can be substantially parallel to each other (for example, when in a flat plan view prior to attachment to the one or more frames, when the tabs are attached to the one or more frames, or both). The second portion can have a half-elliptical or semi-elliptical shape that defines a cusp edge. The cusp edge can extend from the base edge of the first tab to the base edge of the second tab. The cusp edge can be curved along its entire length between the base edges of the first and second tabs. The valvular structure can be coupled to the frame via a plurality of commissure assemblies formed by paired ones of the tabs from adjacent leaflets. A cusp edge portion of the second portion of each leaflet at the respective cusp edge can be directly or indirectly coupled to one or more frames via one or more sutures, and a suture line formed by the one or more sutures can follow a curvature of the cusp edge. The suture line formed by the one or more sutures can be continuous from apices of the cusp edges substantially to the commissure assemblies.

In another aspect, a prosthetic heart valve can be summarized as comprising an annular frame and valvular means for regulating blood flow through the prosthetic heart valve in a hemodynamic situation at an implanted location within a patient that experiences a relatively-low pressure gradient. The annular frame can have an inflow end and an outflow end separated from the inflow end along an axial direction of the frame.

In another aspect, an assembly can be summarized as comprising a delivery apparatus and a prosthetic heart valve. The delivery apparatus can comprise an elongated shaft. The prosthetic heart valve can be according to any of the above noted examples. The prosthetic heart valve can be mounted on the elongated shaft in the radially-compressed configuration for delivery into a patient's body.

In another aspect, a method of implanting a prosthetic heart valve in a patient's body can be summarized as comprising inserting a distal end of a delivery apparatus into vasculature of a patient. The delivery apparatus can comprise an elongated shaft. The prosthetic heart valve can be according to any of the above noted examples and can be releasably mounted in the radially-compressed configuration on the elongated shaft of the delivery apparatus.

In another aspect, a method of assembling a prosthetic heart valve can be summarized as comprising providing an inner skirt over a radially-inner circumferential surface of an annular frame. The annular frame can be radially collapsible and expandable between a radially-compressed configuration and a radially-expanded configuration. The annular frame can have an inflow end and an outflow end separated from the inflow end along an axial direction of the frame. The method can further comprise forming a plurality of commissure assemblies with a plurality of leaflets. Each leaflet can have a first portion, first and second tabs, and a second portion. The first and second tabs can be on opposite sides of the first portion with respect to a centerline of the first portion. Each of the tabs can have a base edge and an outer edge. The outer edges of the tabs can be substantially parallel to each other. The second portion of each leaflet can have a half-elliptical or semi-elliptical shape that defines a cusp edge. The cusp edge of each leaflet can extend from the base edge of the first tab to the base edge of the second tab. The cusp edge of each leaflet can be curved along its entire length between the base edges of the first and second tabs. Each commissure assembly can be formed by paired tabs of adjacent leaflets. The method can also include coupling each commissure assembly to the annular frame, and coupling a cusp edge portion of the second portion of each leaflet at the respective cusp edge to the inner skirt via one or more sutures. A suture line formed by the one or more sutures can follow a curvature of the cusp edge.

In another aspect, a prosthetic heart valve can be summarized as comprising an annular frame, a valvular structure, an inner skirt, and an outer skirt. The annular frame can be radially collapsible and expandable between a radially-compressed configuration and a radially-expanded configuration. The annular frame can have an inflow end and an outflow end separated from the inflow end along an axial direction of the frame. The valvular structure can be supported within the annular frame and can comprise a plurality of leaflets. Each leaflet can have a first portion, a second portion, and first and second tabs. The first and second tabs can be on opposite sides of the first portion with respect to a centerline of the first portion. Each of the tabs can have a base edge and an outer edge. The outer edges of the first and second tabs can be substantially parallel to each other. The second portion can have a half-elliptical or semi-elliptical shape that defines a cusp edge extending from the base edge of the first tab to the base edge of the second tab. The cusp edge can be curved along its entire length between the base edges of the first and second tabs. The inner skirt can be disposed on a radially-inner circumferential surface of the annular frame and coupled thereto. The inner skirt can comprise a hermetic layer constructed such that, when the prosthetic heart valve is implanted in a patient, ingrowth of cells from surrounding native tissue of the patient into the hermetic layer is prevented. The outer skirt can be disposed over a radially-outer circumferential surface of the annular frame. The outer skirt can cover substantially all of the radially-outer circumferential surface of the annular frame between the inflow and outflow ends. The valvular structure can be coupled to the frame via a plurality of commissure assemblies formed by paired ones of the tabs from adjacent leaflets. The inner skirt can be disposed between the annular frame and the second portion of each leaflet along a radial direction of the annular frame. The inner skirt can extend along the axial direction of the frame from at least apices of the cusp edges of the leaflets to at least the plurality of commissure assemblies. The cusp edge portion of the second portion of each leaflet at the respective cusp edge can be coupled to the inner skirt by one or more sutures. A suture line formed by the one or more sutures can follow a curvature of the cusp edge. The suture line can be substantially continuous or piecewise-continuous from the apices of the cusp edges substantially to the commissure assemblies.

In another aspect, a prosthetic heart valve can be summarized as comprising an annular frame and a valvular structure. The annular frame can be radially collapsible and expandable between a radially-compressed configuration and a radially-expanded configuration. The annular frame can have an inflow end and an outflow end separated from the inflow end along an axial direction of the frame. The valvular structure can be supported within the annular frame and can comprise a plurality of leaflets. Each leaflet can have a first portion, a second portion, and first and second tabs. The first and second tabs can be on opposite sides of the first portion with respect to a centerline of the first portion. Each of the tabs can have a base edge and an outer edge. The outer edges of the first and second tabs can be substantially parallel to each other. The second portion can have a half-elliptical or semi-elliptical shape that defines a cusp edge extending from the base edge of the first tab to the base edge of the second tab. The cusp edge can be curved along its entire length between the base edges of the first and second tabs. The annular frame can be encapsulated by a hermetic layer constructed such that, when the prosthetic heart valve is implanted in a patient, ingrowth of cells from surrounding native tissue of the patient into the hermetic layer is prevented. The valvular structure can be coupled to the frame via a plurality of commissure assemblies formed by paired ones of the tabs from adjacent leaflets. A cusp edge portion of the second portion of each leaflet at the respective cusp edge can be coupled to the hermetic layer by one or more sutures. A suture line formed by the one or more sutures can follow a curvature of the cusp edge. The suture line can be substantially continuous or piecewise-continuous from apices of the cusp edges substantially to the commissure assemblies.

In another aspect, a prosthetic heart valve can be summarized as comprising an annular frame, valvular means for regulating blood flow through the prosthetic heart valve in a hemodynamic situation at an implanted location within a patient that experiences a relatively-low pressure gradient, and means for preventing ingrowth of cells from surrounding native tissue of a patient onto leaflets of the valvular means. The annular frame can be radially collapsible and expandable between a radially-compressed configuration and a radially-expanded configuration. The annular frame can have an inflow end and an outflow end separated from the inflow end along an axial direction of the frame.

In another aspect, an assembly can be summarized as comprising a delivery apparatus and a prosthetic heart valve. The delivery apparatus can comprise an elongated shaft. The prosthetic heart valve can be according to any of the above noted examples. The prosthetic heart valve can be mounted on the elongated shaft in the radially-compressed configuration for delivery into a patient's body.

In another aspect, a method of implanting a prosthetic heart valve in a patient's body can be summarized as comprising inserting a distal end of a delivery apparatus into vasculature of a patient. The delivery apparatus can comprise an elongated shaft. The prosthetic heart valve can be according to any of the above noted examples and can be releasably mounted in the radially-compressed configuration on the elongated shaft of the delivery apparatus.

In another aspect, a method of assembling a prosthetic heart valve can be summarized as comprising providing an inner skirt on a radially-inner circumferential surface of an annular frame. The annular frame can be radially collapsible and expandable between a radially-compressed configuration and a radially-expanded configuration. The annular frame can have an inflow end and an outflow end separated from the inflow end along an axial direction of the frame. The inner skirt can comprise a hermetic layer constructed such that, when the prosthetic heart valve is implanted in a patient, ingrowth of cells from surrounding native tissue of the patient into the hermetic layer is prevented. The hermetic layer can comprise a layer formed directly on the radially-inner circumferential surface of the annular frame. The method can further comprise forming a plurality of commissure assemblies with a plurality of leaflets. Each leaflet can have a first portion, first and second tabs, and a second portion. The first and second tabs can be on opposite sides of the first portion with respect to a centerline of the first portion. Each of the tabs can have a base edge and an outer edge. The outer edges of the tabs can be substantially parallel to each other. The second portion of each leaflet can have a half-elliptical or semi-elliptical shape that defines a cusp edge. The cusp edge of each leaflet can extend from the base edge of the first tab to the base edge of the second tab. The cusp edge of each leaflet can be curved along its entire length between the base edges of the first and second tabs. Each commissure assembly can be formed by paired tabs of adjacent leaflets. The method can further comprise coupling each commissure assembly to the annular frame. The inner skirt can be disposed between the annular frame and the second portion of each leaflet along a radial direction of the annular frame, and the inner skirt can extend along the axial direction of the frame from at least apices of the cusp edges of the leaflets to at least the plurality of commissure assemblies. The method can also comprise coupling a cusp edge portion of the second portion of each leaflet at the respective cusp edge to the inner skirt via one or more sutures. A suture line formed by the one or more sutures can follow a curvature of the cusp edge. The suture line can be substantially continuous or piecewise-continuous from apices of the cusp edges substantially to the commissure assemblies.

In another aspect, a method of assembling a prosthetic heart valve can be summarized as comprising coupling an inner skirt to a radially-inner circumferential surface of an annular frame. The annular frame can be radially collapsible and expandable between a radially-compressed configuration and a radially-expanded configuration. The annular frame can have an inflow end and an outflow end separated from the inflow end along an axial direction of the frame. The inner skirt can comprise a hermetic layer constructed such that, when the prosthetic heart valve is implanted in a patient, ingrowth of cells from surrounding native tissue of the patient into the hermetic layer is prevented. The method can further comprise forming a plurality of commissure assemblies with a plurality of leaflets. Each leaflet can have a first portion, first and second tabs, and a second portion. The first and second tabs can be on opposite sides of the first portion with respect to a centerline of the first portion. Each of the tabs can have a base edge and an outer edge. The outer edges of the tabs can be substantially parallel to each other. The second portion of each leaflet can have a half-elliptical or semi-elliptical shape that defines a cusp edge. The cusp edge of each leaflet can extend from the base edge of the first tab to the base edge of the second tab. The cusp edge of each leaflet can be curved along its entire length between the base edges of the first and second tabs. Each commissure assembly can be formed by paired tabs of adjacent leaflets. The method can also comprise coupling each commissure assembly to the annular frame. The inner skirt can be disposed between the annular frame and the second portion of each leaflet along a radial direction of the annular frame, and the inner skirt can extend along the axial direction of the frame from at least apices of the cusp edges of the leaflets to at least the plurality of commissure assemblies. The method can further comprise coupling a cusp edge portion of the second portion of each leaflet at the respective cusp edge to the inner skirt via one or more sutures. A suture line formed by the one or more sutures can follow a curvature of the cusp edge, and the suture line can be substantially continuous or piecewise-continuous from apices of the cusp edges substantially to the commissure assemblies.

In another aspect, a method of assembling a prosthetic heart valve can be summarized as comprising encapsulating an annular frame with a hermetic layer. The annular frame can be radially collapsible and expandable between a radially-compressed configuration and a radially-expanded configuration. The annular frame can have an inflow end and an outflow end separated from the inflow end along an axial direction of the frame. The hermetic layer can be constructed such that, when the prosthetic heart valve is implanted in a patient, ingrowth of cells from surrounding native tissue of the patient into the hermetic layer is prevented. The method can further comprise forming a plurality of commissure assemblies with a plurality of leaflets. Each leaflet can have a first portion, first and second tabs, and a second portion. The first and second tabs can be on opposite sides of the first portion with respect to a centerline of the first portion. Each of the tabs can have a base edge and an outer edge. The outer edges of the tabs can be substantially parallel to each other. The second portion of each leaflet can have a half-elliptical or semi-elliptical shape that defines a cusp edge. The cusp edge of each leaflet can extend from the base edge of the first tab to the base edge of the second tab. The cusp edge of each leaflet can be curved along its entire length between the base edges of the first and second tabs. Each commissure assembly can be formed by paired tabs of adjacent leaflets. The method can further comprise coupling each commissure assembly to the annular frame, and coupling a cusp edge portion of the second portion of each leaflet at the respective cusp edge to the hermetic layer via one or more sutures. A suture line formed by the one or more sutures can follow a curvature of the cusp edge. The suture line can be substantially continuous or piecewise-continuous from apices of the cusp edges substantially to the commissure assemblies.

### BRIEF DESCRIPTION OF THE DRAWINGS

FIG. 1 shows a schematic cross-sectional view of a human heart in which a prosthetic heart valve may be installed, according to one or more examples of the disclosed subject matter.

FIG. 2A shows a partial cross-sectional view of an exemplary prosthetic heart valve installed in the aortic position of a human heart.

FIGS. 2B-2C show a valvular structure of the implanted prosthetic heart valve of FIG. 2A in closed and open configurations, respectively, as viewed from an outflow end of the prosthetic heart valve.

FIG. 2D is a simplified cross-sectional view of the implanted prosthetic heart valve of FIG. 2C.

FIGS. 3A-3D are simplified cross-sectional views of implanted prosthetic heart valves employing inner hermetic layers for preventing ingrowth of cells from surrounding native tissue, according to one or more examples of the disclosed subject matter.

FIG. 3E is a simplified close-up perspective view illustrating an exemplary coupling of a commissure assembly to an inner skirt, corresponding to the configuration of FIG. 3D.

FIGS. 4A-4D are simplified cross-sectional views of implanted prosthetic heart valves employing inner and outer hermetic layers for preventing ingrowth of cells from surrounding native tissue, according to one or more examples of the disclosed subject matter.

FIG. 5A is a side view of a first exemplary prosthetic heart valve having an inner skirt comprising a hermetic layer for preventing ingrowth of cells from surrounding native tissue, according to one or more examples of the disclosed subject matter.

FIG. 5B is a perspective view of an interior of the prosthetic heart valve of FIG. 5A as viewed from an inflow end of the valve.

FIG. 5C is a perspective view from an outflow end of the first exemplary prosthetic heart valve of FIG. 5A with an outer skirt installed.

FIG. 5D is a perspective view from an outflow end of an annular frame for the first exemplary prosthetic heart valve of FIG. 5A.

FIG. 5E is a close-up perspective view of a commissure attached to the annular frame of the first exemplary prosthetic heart valve of FIG. 5A.

FIGS. 6A-6B are a side view and a perspective view from an outflow end, respectively, of a second exemplary prosthetic heart valve having inner and outer skirts comprising hermetic layers for preventing ingrowth of cells from surrounding native tissue, according to one or more examples of the disclosed subject matter.

FIG. 6C is a simplified cross-sectional view of a commissure attached to the annular frame of the second exemplary prosthetic heart valve of FIG. 6A.

FIG. 7A is a side view of a third exemplary prosthetic heart valve having an inner skirt comprising a hermetic layer for preventing ingrowth of cells from surrounding native tissue, according to one or more examples of the disclosed subject matter.

FIG. 7B is a perspective view of an interior of the prosthetic heart valve of FIG. 7A as viewed from an inflow end of the valve.

FIG. 7C is a simplified cross-sectional view illustrating arrangement of the inner and outer skirts of the third exemplary prosthetic heart valve of FIG. 7A.

FIG. 8A is a perspective view from an outflow end of a fourth exemplary prosthetic heart valve having an inner skirt comprising a hermetic layer for preventing ingrowth of cells from surrounding native tissue, according to one or more examples of the disclosed subject matter.

FIG. 8B is a semi-transparent view of the fourth exemplary prosthetic heart valve of FIG. 8A illustrating the underlying structural features of the valve frame.

FIGS. 9A-9B are a side view and a perspective view from the outflow end, respectively, of a fifth exemplary prosthetic heart valve having a frame encapsulated in a hermetic layer for preventing ingrowth of cells from surrounding native tissue, according to one or more examples of the disclosed subject matter.

FIGS. 10A-10B is a side view and a perspective view from an outflow end, respectively, of a sixth exemplary prosthetic heart valve having hermetic layers for preventing ingrowth of cells from surrounding native tissue, according to one or more examples of the disclosed subject matter.

FIG. 10C is a simplified cross-sectional view of the sixth exemplary prosthetic heart valve of FIG. 10A.

FIG. 11A is a perspective view from an outflow end of a seventh exemplary prosthetic heart valve having hermetic layers for preventing ingrowth of cells from surrounding native tissue, according to one or more examples of the disclosed subject matter.

FIG. 11B is a partial cross-sectional view of the seventh exemplary prosthetic heart valve of FIG. 11A illustrating the underlying structural features of the valve frame.

FIGS. 12A-12B are a simplified cross-sectional view and a view from an outflow end, respectively, of an implanted prosthetic heart valve at a low-pressure gradient location.

FIG. 13A is a simplified plan view of a leaflet for use in an exemplary valvular structure for regulating blood flow in a low-pressure gradient hemodynamic situation, according to one or more examples of the disclosed subject matter.

FIGS. 13B-13C are close-up exterior and interior views, respectively, of the exemplary valvular structure coupled to an annular frame and inner skirts of a prosthetic heart valve, according to one or more examples of the disclosed subject matter.

FIG. 14A is a side view of a first exemplary prosthetic heart valve having a valvular structure for regulating blood flow in a low-pressure gradient hemodynamic situation, according to one or more examples of the disclosed subject matter.

FIG. 14B is a perspective view from an outflow end of an annular frame for the first exemplary prosthetic heart valve of FIG. 14A.

FIG. 14C is a simplified plan view of a single leaflet from the valvular structure of the first exemplary prosthetic heart valve of FIG. 14A.

FIG. 14D is a close-up perspective view of a commissure attached to the annular frame of the first exemplary prosthetic heart valve of FIG. 14A.

FIG. 14E is a view from an outflow end of the valvular structure of the first exemplary prosthetic heart valve of FIG. 14A in an open configuration.

FIG. 15A is a simplified plan view of a single leaflet from a valvular structure from a comparative example of a prosthetic heart valve.

FIG. 15B is a view from an outflow end of the valvular structure of the comparative example of a prosthetic heart valve of FIG. 15A in an open configuration.

FIG. 16A is a side view of an exemplary docking station for a prosthetic heart valve having a valvular structure for regulating blood flow in a low-pressure gradient hemodynamic situation, according to one or more examples of the disclosed subject matter.

FIG. 16B is a partial cross-sectional view of the docking station of FIG. 16A with prosthetic heart valve installed therein.

FIG. 17A is a simplified cross-sectional view of an exemplary prosthetic mitral valve that has an annular frame encapsulated in a hermetic layer for preventing ingrowth of cells from surrounding native tissue, according to one or more examples of the disclosed subject matter.

FIG. 17B is a perspective view from an outflow end of the exemplary prosthetic mitral valve of FIG. 17A.

FIGS. 17C-17D are a semi-transparent side view and semi-transparent perspective view from an outflow end, respectively, of the exemplary prosthetic mitral valve of FIG. 17B with outer skirt removed for illustration of the underlying features.

FIG. 18 is a simplified view of an exemplary delivery system for implanting any of the exemplary prosthetic heart valves within a patient, according to one or more examples of the disclosed subject matter.

FIG. 19 is a simplified view of an exemplary docking station into which the exemplary prosthetic mitral valve can be mounted within a patient, according to one or more examples of the disclosed subject matter.

FIGS. 20A-20B are a partial cross-sectional view and a view from an inflow end, respectively, of an initial stage of an exemplary implantation of the docking station of FIG. 19 within a native mitral valve of a patient.

FIGS. 20C-20D are a partial cross-sectional view and a view from an inflow end, respectively, of a subsequent stage of the exemplary implantation of the docking station of FIG. 19 within a native mitral valve of a patient.

FIG. 21A is a partial cross-sectional view of an exemplary implantation of the exemplary prosthetic mitral valve of FIG. 17B at the previously implanted docking station.

FIG. 21B is a simplified view of the exemplary prosthetic mitral valve of FIG. 17B after implantation within the native mitral valve of the patient as viewed from the left ventricle.

FIG. 22 is a perspective view of an outer skirt for a prosthetic heart valve, depicted in an axially elongated state.

FIG. 23 illustrates the outer skirt of FIG. 22 in a radially expanded state.

FIG. 24A is a plan view of an exemplary woven fabric for a first fabric layer of the outer skirt of FIGS. 22 and 23, depicted in an elongated state.

FIG. 24B is a detail of circled region 24B in FIG. 24A.

FIG. 25 illustrates the woven fabric of FIG. 24 in a radially expanded state.

FIG. 26 illustrates a basic leno weave structure.

FIGS. 27A and 27B illustrate forming of a first fabric layer of the outer skirt of FIGS. 22 and 23.

FIG. 28 illustrates a basic plain weave structure.

FIGS. 29A, 29B, and 30 illustrate forming of a second fabric layer of the outer skirt of FIGS. 22 and 23.

FIG. 31 is an elevated view showing the outer skirt of FIG. 23 disposed around a radially expanded prosthetic heart valve.

FIG. 32 is an elevated view showing the inner fabric layer of the outer skirt of FIG. 23 disposed around a frame of a prosthetic heart valve.

FIG. 33 is a cross-sectional view of the outer skirt of FIG. 23 disposed around a prosthetic heart valve.

FIG. 34 illustrates the outer skirt of FIG. 23 disposed around a prosthetic heart valve with flaps of the inner fabric layer folded around the outer layer.

### DETAILED DESCRIPTION

General Considerations

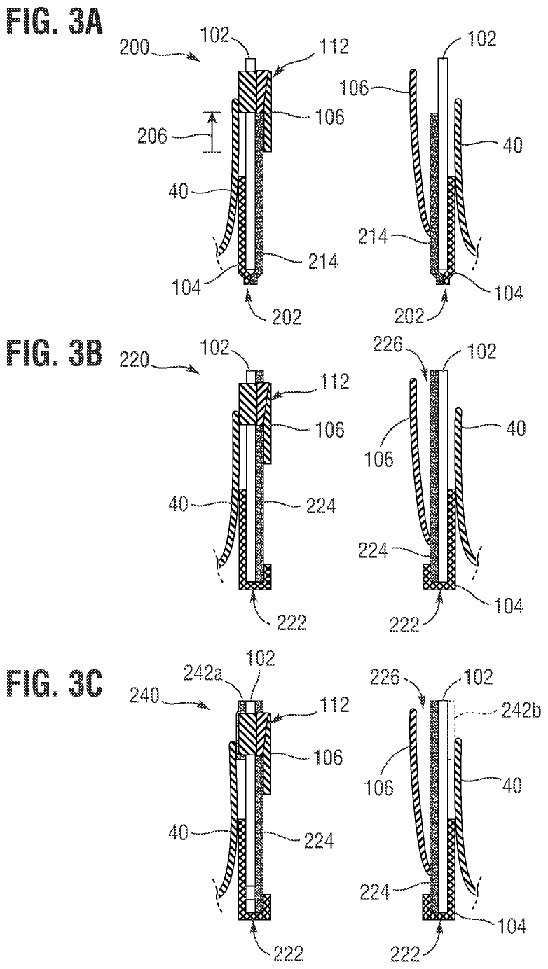

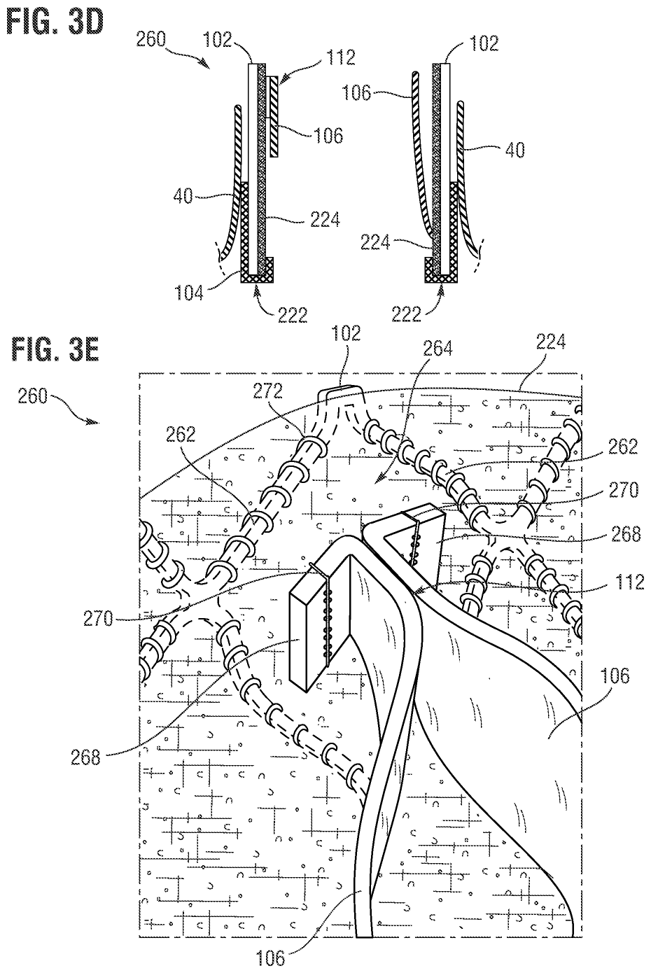

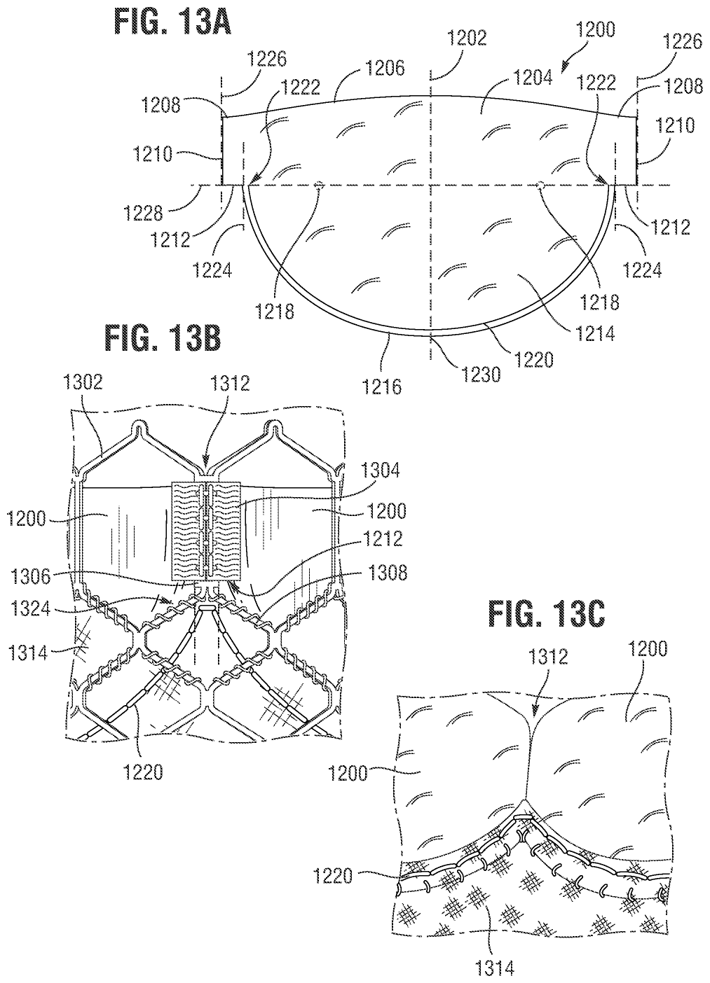

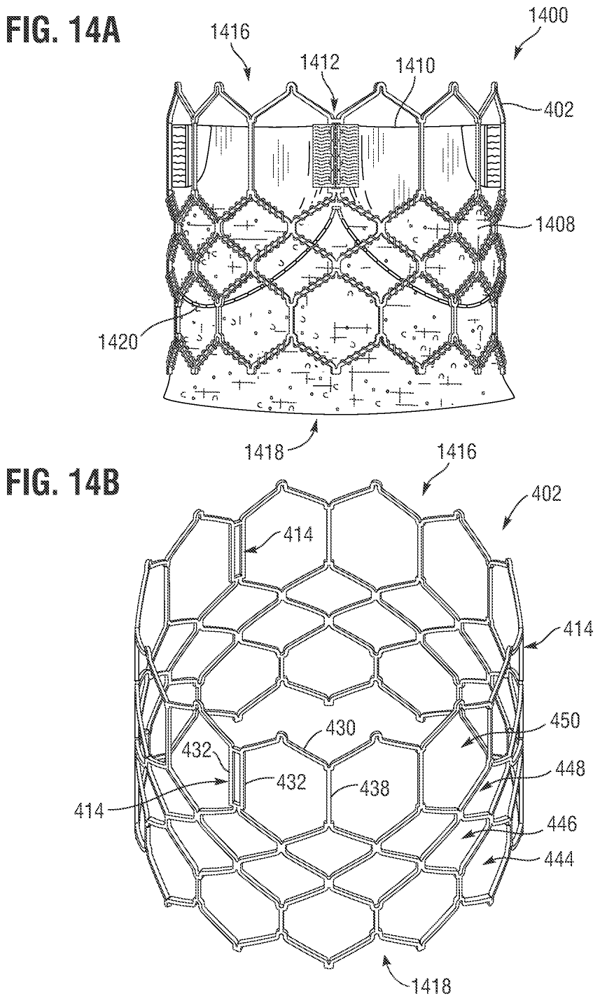

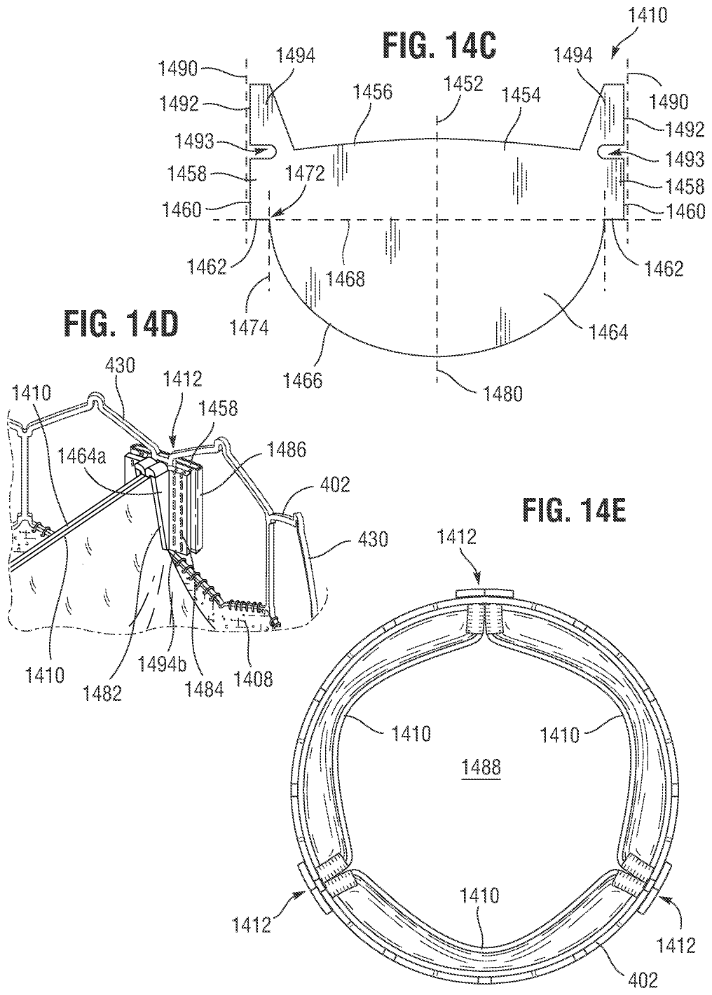

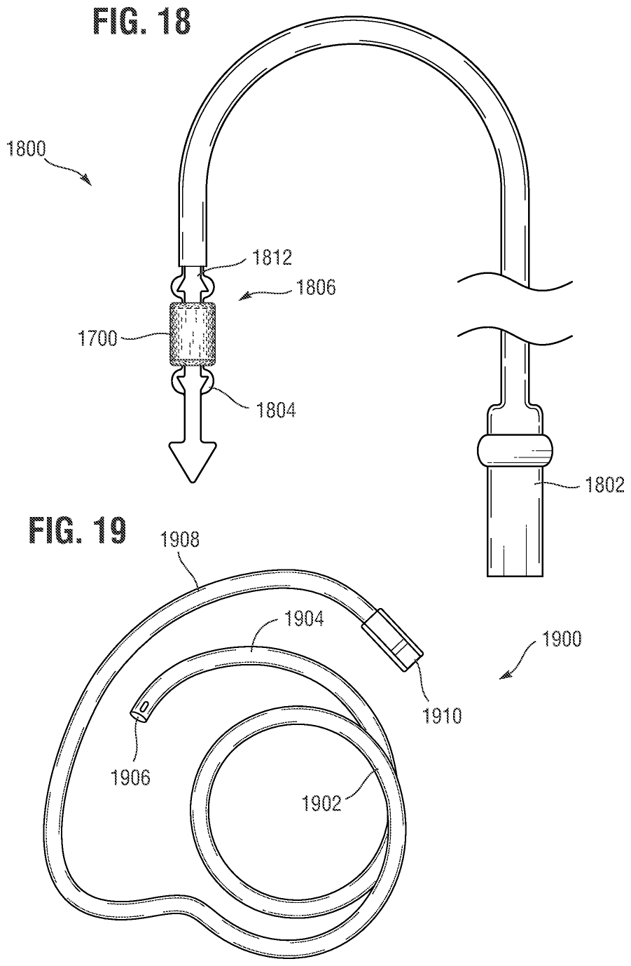

All features described herein are independent of one another and, except where structurally impossible, can be used in combination with any other feature described herein. For example, a delivery apparatus 1800 as shown in FIG. 18 can be used in combination with any of the prosthetic heart valves described herein. In another example, the valvular structures, shown in and described with respect to FIGS. 13A-14E, can be used in combination with any of the prosthetic valves shown in FIGS. 3A-9B. In still another example, the various exemplary configurations of hermetic layers for the prosthetic heart valve frames, protective coverings, and/or coupling members, as discussed with respect to FIGS. 3A-11B can be used with any of the disclosed prosthetic valves or variations thereof.

For purposes of this description, certain aspects, advantages, and novel features of the subject matter are described herein. The disclosed methods, apparatus, and systems should not be construed as being limiting in any way. Instead, the present disclosure is directed toward all novel and nonobvious features and aspects of the various disclosed examples and implementations, alone and in various combinations and sub-combinations with one another. The methods, apparatus, and systems are not limited to any specific aspect or feature or combination thereof, nor do the disclosed examples and implementations require that any one or more specific advantages be present, or problems be solved. The technologies from any example or implementation can be combined with the technologies described in any one or more of the other examples or implementations.

Although the operations of some of the disclosed examples and implementations are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used herein with reference to the prosthetic heart valve assembly and implantation and structures of the prosthetic heart valve, “proximal” refers to a position, direction, or portion of a component that is closer to the user and a handle of the delivery system or apparatus that is outside the patient, while “distal” refers to a position, direction, or portion of a component that is further away from the user and the handle, and closer to the implantation site. The terms “longitudinal” and “axial” refer to an axis extending in the proximal and distal directions, unless otherwise expressly defined.

The terms “axial direction,” “radial direction,” and “circumferential direction” have been used herein to describe the arrangement and assembly of components relative to the geometry of the frame of the prosthetic heart valve. Such terms have been used for convenient description, but the disclosed examples and implementations are not strictly limited to the description. In particular, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

As used herein, the terms “integrally formed” and “unitary construction” refer to a construction that does not include any welds, fasteners, or other means for securing separately formed pieces of material to each other.

As used herein, operations that occur “simultaneously” or “concurrently” occur generally at the same time as one another, although delays in the occurrence of operation relative to the other due to, for example, spacing between components, are expressly within the scope of the above terms, absent specific contrary language.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language. As used herein, “and/or” means “and” or “or,” as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,” “top,” “bottom,” “interior,” “exterior,” “left,” “right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

Introduction to the Disclosed Technology

Described herein are prosthetic heart valves designed to reduce the risk of thrombosis. In some implementations, the prosthetic heart valve can have one or more hermetic layers constructed to prevent ingrowth of surrounding native tissue. For example, the inner skirt and/or the outer skirt can comprise one or more hermetic layers, or the entire valve frame can be encapsulated within one or more hermetic layers. Whereas conventional materials employed for inner and outer skirts can have pores therein that allow ingrowth of surrounding native tissue, the hermetic layers disclosed herein can be formed of a hydrophobic material and can be substantially nonporous or otherwise have pores of sufficiently small size that discourage cellular ingrowth. The disclosed hermetic layers can thus prevent, or at least reduce, the ingrowth of the surrounding native tissue and thereby avoid, or at least reduce, pannus formation on the prosthetic valve leaflets that may have otherwise resulted from such ingrowth. Alternatively or additionally, in some implementations, shapes of the leaflets of the valvular structure of the prosthetic valve and/or the coupling of the leaflets to the frame can be selected to avoid, or at least reduce, the incidence of stasis when implanted at a relatively-low pressure gradient hemodynamic location.

Examples of the Disclosed Technology

### Exemplary Hermetic Layers to Prevent Tissue Ingrowth

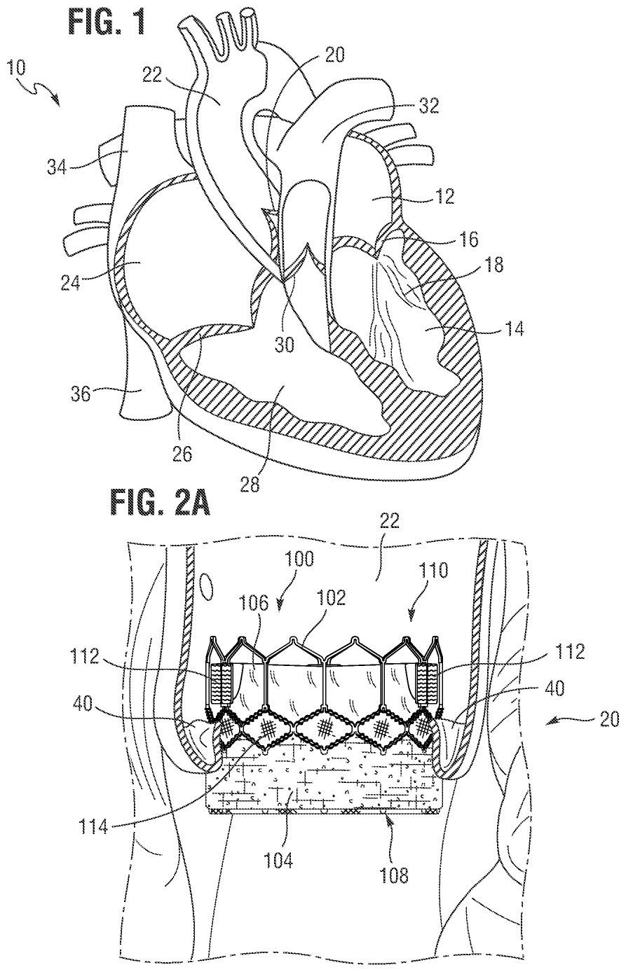

Referring to FIG. 1, a schematic cross-sectional view of a human heart 10 is shown. The mitral valve 16 separates the left ventricle 14 from the left atrium 12, and the tricuspid valve 26 separates the right ventricle 28 from the right atrium 24. The aortic valve 20 further separates the left ventricle 14 from the ascending aorta 22, and the pulmonary valve 30 further separates the right ventricle 28 from the pulmonary artery 32. Deoxygenated blood is delivered to the right atrium 24 by the superior vena cava 34, the inferior vena cava 36, and the coronary sinus. During the diastolic phase, as the right ventricle 28 expands, deoxygenated blood in the right atrium 24 is directed through the tricuspid valve 26 into the right ventricle 28. In the subsequent systolic phase, contraction by the right ventricle 28 forces the deoxygenated blood therein through the pulmonary valve 30 into the pulmonary artery 32. In addition to forcing blood through the one-way pulmonary valve 30, the pressure of the contraction by the right ventricle 28 also urges the one-way tricuspid valve 26 closed, thereby preventing blood in the right ventricle 28 from re-entering the right atrium 24.

Oxygenated blood is delivered to the left atrium 12 by the pulmonary veins. During the diastolic phase, as the left ventricle 14 expands, the oxygenated blood in the left atrium 12 is directed through the mitral valve 16 into the left ventricle 14. In the subsequent systolic phase, contraction by the left ventricle 14 forces the oxygenated blood through the aortic valve 20 into the ascending aorta 22 for circulation through the body. In addition, forcing the blood through the one-way aortic valve 20, the pressure of the contraction by the left ventricle 14 also urges the one-way mitral valve 16 closed, thereby preventing blood in the left ventricle 14 from re-entering the left atrium 12. The contraction by the left ventricle 14 generates a significant pressure differential between the left ventricle 14 and the left atrium 12. A series of chordae tendineae 18 connect leaflets of the mitral valve 16 to papillary muscles located on the walls of the left ventricle 14. During the diastolic phase, both the chordae tendineae 18 and the papillary muscles are tensioned to hold the leaflets of the mitral valve 16 in the closed position and to prevent the leaflets from extending backward into the left atrium 12.

Any of the above noted native heart valves may fail to operate properly, for example, by allowing blood to backflow therethrough or regurgitate into an upstream heart chamber or blood vessel. In some implementations, a prosthetic heart valve can be implanted within the native heart valve to help prevent or inhibit such regurgitation and/or to address any other insufficiency of the native heart valve. In other implementations, a prosthetic heart valve can be implanted in a blood vessel that leads to a heart chamber, for example, the inferior vena cava or the superior vena cava, to prevent or inhibit backflow therein during the systolic phase. In general, prosthetic heart valves implanted at the aortic position (e.g., within the native aortic valve 20) can normally experience relatively-high pressure gradients (e.g., driving pressure of about 125 mmHg). In contrast, prosthetic heart valves implanted at the pulmonary position (e.g., within the native pulmonary valve 30), the tricuspid position (e.g., within the native tricuspid valve 26), the mitral position (e.g., within the native mitral valve 16), or blood vessels leading to heart chambers (e.g., within the inferior vena cava 36 or superior vena cava 34) can normally experience relatively-low pressure gradients (e.g., driving pressure of about 30 mmHg or less).

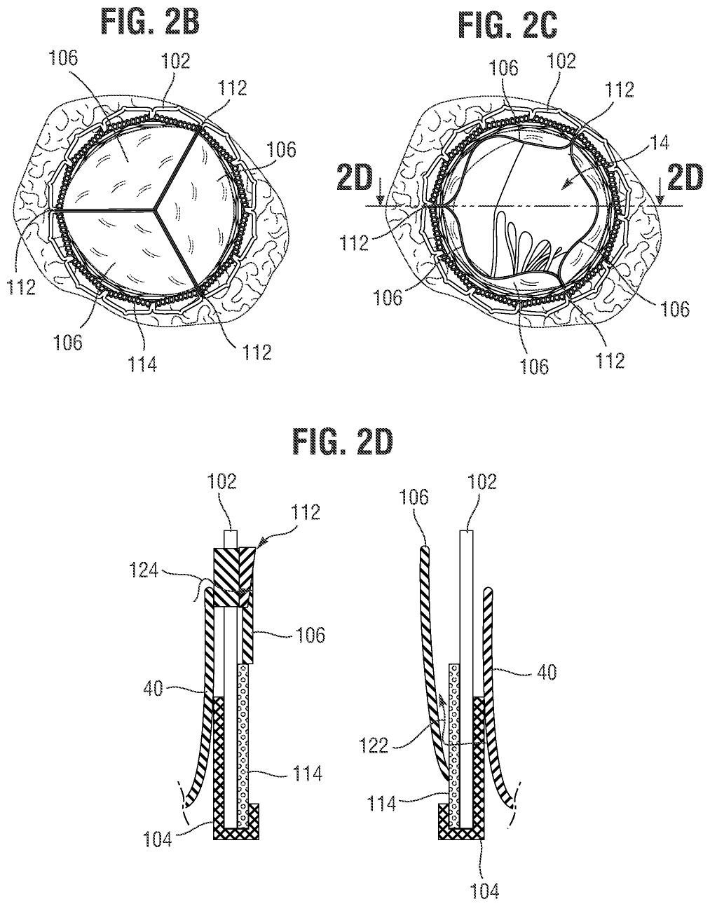

FIGS. 2A-2D illustrate a prosthetic heart valve 100 that has been implanted at the aortic position (e.g., within leaflets 40 of the native aortic valve 20). The prosthetic heart valve 100 includes an annular frame 102 and a valvular structure of three leaflets 106. The valvular structure is coupled to the annular frame 102 near an outflow end 110 of the prosthetic valve 100 by a plurality of commissures 112 formed by tabs of adjacent leaflets 106. The prosthetic heart valve 100 also includes an inner skirt 114 that covers an inner circumferential surface of the annular frame 102 near the inflow end 108 of the prosthetic valve 100. The prosthetic valve 100 further includes an outer skirt 104 that covers an outer circumferential surface of the annular frame 102 near the inflow end 108. Each leaflet 106 has a cusp edge that is attached to the inner skirt 114. Further details of the prosthetic heart valve 100 are disclosed in U.S. Publication No. 2019/0365530, which is incorporated herein by reference.

In conventional prosthetic heart valves, the inner skirt 114 and the outer skirt 104 are constructed of a porous material, such as woven, braided or knitted fabrics formed from synthetic fibers, such as polyethylene terephthalate (PET) fibers. The porous nature of the skirt material (e.g., having pores greater than 30-50 μm) is designed to encourage cellular ingrowth of the surrounding native tissue while otherwise being substantially impermeable to blood cells in the blood flow through the heart valve. For example, tissue or cells from leaflets 40 that are in contact with outer skirt 104 can grow into the outer skirt 104 and thereby to the inner skirt 114 on the opposite side of the annular frame 102. This native tissue ingrowth into skirts 104, 114 can further secure the implanted heart valve 100 within the native anatomy of the patient and reduce paravalvular leakage (PVL).

However, the ingrowth of the surrounding tissue into the prosthetic heart valve 100 may proceed beyond just the outer skirt 104 and the inner skirt 114. Since the leaflets 106 of the prosthetic valve 100 are attached to the inner skirt 114, any tissue ingrowth into the inner skirt 114 can migrate onto leaflet 106, as shown by route 122 in FIG. 2D. In addition, since the commissures 112 of the valvular structure of the prosthetic valve 100 have a portion disposed external to the annular frame 102, the commissures 112 are susceptible to contact with the native anatomy (e.g., leaflets 40), which can offer an additional route 124 for tissue growth onto the leaflets. The tissue infiltration into the valvular structure can lead to the formation of pannus on the surfaces of the leaflets 106, which may impede functionality of the valvular structure. Moreover, the pannus on the leaflets 106 can serve as substrates onto which thrombi may later deposit. When implanted at relatively-low pressure gradient locations, the lower flow conditions experienced by the prosthetic valve 100 may allow for easier propagation of the tissue onto the leaflets 106 via routes 122, 124.

Accordingly, in some implementations, a prosthetic heart valve is provided with one or more hermetic layers to prevent, or at least reduce, the ingrowth of native tissue via route 122 and/or route 124. As used herein, “hermetic layer” refers to a layer that is constructed such that, when implanted in a patient, ingrowth of cells into the layer is prevented or at least discouraged. In some implementations, such a hermetic layer is substantially nonporous or otherwise has pores therein of sufficiently small size to discourage cellular ingrowth. The size and characteristics of pores within the hermetic layer (e.g., porosity, tortuosity) can be adapted to prevent cellular ingrowth, which may also depend on implant location (e.g., pressure gradient, blood flow conditions), desired implant lifetime, hermetic layer thickness, and/or other factors.

In some implementations, a hermetic layer can have pores therein that are each no greater than, for example, 8 μm in size (e.g., in diameter or, if non-circular in shape, in a maximum lateral dimension). Alternatively, or additionally, in some implementations, a size of each pore in the hermetic layer can be, for example, 20 μm or less, 10 μm or less, or 5 μm or less. In some implementations, the hermetic layer may have a range of different pore sizes, and the distribution of pore sizes may be such that at least 90% of the pores have a size of 8 μm or less. Alternatively or additionally, in some implementations, the distribution of pore sizes in the hermetic layer may be such that at least 90% of the pores have a size of 20 or less, 10 μm or less, or 5 μm or less.

In some implementations, pores that open to an outer-diameter side of the hermetic layer (e.g., a side facing outward toward the surrounding native tissue) can have sizes greater than that of pores that open to an inner-diameter side of the hermetic layer (e.g., a side facing inward toward the valvular structure of the prosthetic valve). In such implementations, the pores opening to the inner-diameter side of the hermetic layer can each be no greater than, for example, 8 μm in size. Alternatively, or additionally, in some implementations, a size of each individual pore opening to the inner-diameter side of the hermetic layer can be, for example, 20 μm or less, 10 μm or less, or 5 μm or less. Alternatively, or additionally, in some implementations, the inner-diameter side of the hermetic layer may have a distribution of pore sizes with at least 90% of the pores having a size of 20 μm or less, 10 μm or less, 8 inn or less, or 5 μm or less.

In some implementations, the pore sizes of the hermetic layer can be characterized by imaging the pores in a portion of the hermetic layer or in an entirety of the hermetic layer. Alternatively or additionally, in some implementations, the pore sizes of the hermetic layer can be characterized by imaging the pores on at least a portion of the inner-diameter side of the hermetic layer. For example, the pore sizes of the hermetic layer can be characterized by optical microscopy, electron microscopy (e.g., scanning electron microscopy), or X-ray micro-computed-tomography (micro-CT) imaging (e.g., American Society for Testing and Materials (ASTM) F2450-18, Standard Guide for Assessing Microstructure of Polymeric Scaffolds for Use in Tissue-Engineered Medical Products, ASTM International, West Conshohocken, Pa., 2018, which is incorporated herein by reference). Alternatively, or additionally, in some implementations, the pore sizes of the hermetic layer can be characterized by performing one or more porometry or porosimetry tests on the hermetic layer. For example, the pore sizes of the hermetic layer can be characterized by capillary flow porometry, bubble point testing (e.g., ASTM F316-03(2019) Standard Test Methods for Pore Size Characteristics of Membrane Filters by Bubble Point and Mean Flow Pore Test, ASTM International, West Conshohocken, Pa., 2019, which is incorporated herein by reference), or mercury intrusion porosimetry (e.g., UOP578-11, Automated Pore Volume and Pore Size Distribution of Porous Substances by Mercury Porosimetry, ASTM International, West Conshohocken, Pa., 2011, or U.S. Pharmacopeial Convention for Micromeritics and Particulate Systems Instruments <267>, Porosimetry by Mercury Intrusion, U.S. Pharmacopeial Convention, Rockville, Md., 2012, both of which are incorporated herein by reference).

In some implementations, the pore size of the hermetic layer can be characterized by the size of particles restricted from passing through the hermetic layer. A nominal pore size of the hermetic layer can be defined as a particle size (e.g., cross-sectional dimension) for which 90% of particles are restricted from passing through the hermetic layer, while an absolute pore size of the hermetic layer can be defined as a maximum particle size for which no particles are able to pass through the hermetic layer for a given testing condition (e.g., a pressure induced across the hermetic layer). In some implementations, the hermetic layer can have a nominal pore size of 20 μm or less, 10 μm or less, 8 μm or less, or even 5 μm or less. Alternatively, or additionally, in some implementations, the hermetic layer can have an absolute pore size of 20 μm or less, 10 μm or less, 8 μm or less, or even 5 μm or less, when subjected to a pressure differential similar to that experienced at the desired implantation location (e.g., in a range of 20-250 mmHg).

In some implementations, the hermetic layer comprises a hydrophobic polymer material. Exemplary materials for the hydrophobic polymer material can include, but are not limited to, polytetrafluoroethylene (PTFE), expanded PTFE (ePTFE), urethane, polyurethane (PU), thermoplastic PU (TPU), silicone, or combinations or copolymers thereof. In one example, the hydrophobic polymer material comprises electrospun urethane layers with ePTFE (such as Bioweb™, sold by Zeus Industrial Products Inc., Orangeburg, S.C.). In another example, the hydrophobic polymer material comprises a copolymer of silicone and TPU (such as Quadrasil™, sold by Biomerics, Salt Lake City, Utah). Other hydrophobic polymer materials or combinations thereof that are not specifically listed above, but are otherwise capable of forming a hermetic later that is substantially nonporous or has sufficiently small size pores that discourage cellular ingrowth, are also possible in some implementations.

In some implementations, the hermetic layer can be a single layer of the hydrophobic polymer material. Alternatively, the hermetic layer can be a laminated structure of multiple sublayers, one or more of which is formed of the hydrophobic polymer material. In one example, the hermetic layer can have a hydrophobic polymer material layer coupled to or formed on a base material layer (e.g., a woven or knitted material, such as PET). In another example, the hydrophobic polymer material may cover the base material layer on at least two sides (e.g., using separate sublayers of hydrophobic polymer material that sandwich the base material layer therebetween, or by encapsulating the base material layer within the hydrophobic polymer material). Alternatively, the hydrophobic polymer material may be provided on only one side of the base material layer, for example, over a surface of the base material layer that faces the frame of the prosthetic heart valve. Although the base material layer may be porous or otherwise have a structure that allows tissue ingrowth, the addition of the hydrophobic polymer material can impart to the base material layer properties that discourage cellular ingrowth, for example, by at least partially filling pores of the base material layer or otherwise obstructing cellular ingress to or egress from the base material layer, thereby allowing the combination to function as a hermetic layer.

In some implementations, the hermetic layer can be directly formed on or over (e.g., with one or more intervening layers) a target barrier surface (e.g., a surface of the prosthetic valve frame or a surface of the base material layer). For example, the hermetic layer can be formed on or over the target barrier surface of the frame by electrospinning, dip coating, or spray coating. In dip coating or spray coating, a hydrophobic polymer material, or a precursor thereof, can be dissolved in a reagent or melted to form a liquid. The resulting liquid is coated on or over the target barrier surface of the frame (e.g., by dipping the frame or base material layer into the liquid or by spraying the liquid onto the frame or base material). By drying the coating or otherwise allowing it to solidify, the hermetic layer can be formed in situ. In electrospinning, the hydrophobic polymer material, or a precursor thereof, is melted or provided in solution. The melt or solution is then charged and ejected through a spinneret under a high-voltage electric field. The ejected melt or solution solidifies or coagulates to form an ultrafine filament, which can be directly deposited onto or over the target barrier surface. In some implementations, the hermetic layer can be formed and then attached to or over (e.g., with one or more intervening layers) the target barrier surface. For example, the hermetic layer can be formed by extrusion or casting and then attached to or over the target barrier surface (e.g., using one or more sutures, by partially embedding the frame or base material layer within the hermetic layer, or by any other attachment means).

Alternatively, or additionally, in some implementations, the annular frame can be encapsulated by the hermetic layer, such that inner and outer surfaces of the frame are covered by the hermetic layer. For example, the frame can be encapsulated by providing separate polymer layers on the radially-inner and radially-outer surfaces of the annular frame and then pressing or melting the polymer layers together to embed the frame therein. In another example, the frame can be encapsulated by coating (e.g., dip or spray) or electrospinning the hermetic layer on all surfaces of the annular frame. Alternatively, or additionally, in some implementations, the base material layer can be encapsulated by a hydrophobic polymer material to form the hermetic layer, such that opposite surfaces of the base material layer are covered by the hydrophobic polymer material. For example, the base material layer can be encapsulated by providing separate polymer layers on opposite surfaces of the base material layer and then pressing or melting the polymer layers together to embed the base material layer therein. In another example, the base material layer can be encapsulated by coating (e.g., dip or spray) or electrospinning the hydrophobic polymer material on all surfaces of the base material layer. Further details regarding options and techniques for fabrication of layers that can be used to form the hermetic layer can be found in U.S. Patent Application Publication No. 2020/0155306 and U.S. Pat. No. 10,232,564, both of which are incorporated by reference herein in their entireties.

In some implementations, at least the inner skirt of the prosthetic heart valve comprises a hermetic layer. For example, FIG. 3A shows an exemplary configuration of a prosthetic heart valve 200 where the inner skirt 214 comprises a hermetic layer. Leaflets 106 of the valvular structure of the prosthetic heart valve 200 can be coupled to the inner skirt 214 (e.g., via one or more sutures), which in turn can be coupled to the frame 102 (e.g., by being formed on the frame or via one or more sutures). The inner skirt 214 thus assists in securing the valvular structure to the frame and provides a seal between the valve and the native annulus by blocking the flow of blood through the open cells of the frame below the lower edge of the leaflets 106. The outer skirt 104 can also be coupled to the frame 102 and may optionally be coupled to the inner skirt 214, for example, via facing portions at the inflow end 202 of the valve 200 or as described for other exemplary configurations below.

In some implementations, the inner skirt 214 with hermetic layer is extended (as shown at 206) to at least the commissures 112, thereby providing a barrier on a radially-inner side of the annular frame where ingrowth of the surrounding native tissue is likely to occur due to contact with the native leaflets 40. In the illustrated example of FIG. 3A, the outer skirt 104 may be formed of a conventional material (e.g., PET), and thus tissue ingrowth may occur into the outer skirt 104. However, the provision of the hermetic layer on the radially-inner side of the annular frame 102 can prevent, or at least inhibit, tissue growth from reaching the leaflets 106 of the prosthetic heart valve 200.

In some implementations, the inner skirt comprising the hermetic layer can be further extended to the outflow end of the prosthetic heart valve. For example, FIG. 3B shows an exemplary configuration of a prosthetic heart valve 220 where the inner skirt 224 with hermetic layer extends from the inflow end 222 to the outflow end 226 of the valve, thereby providing a continuous barrier to tissue ingrowth over an entire radially-inner circumferential surface of annular frame 102. In order to mount the commissures 112 to the annular frame 102, one or more openings can be formed in the inner skirt 224 at locations corresponding to commissure windows of the annular frame 102. The commissures 112 can extend radially through the openings in the inner skirt 224 and through the commissure windows for coupling (e.g., via one or more sutures) on a radially-outer side of the annular frame 102.

Similar to the configuration of FIG. 3A, the prosthetic heart valve 220 of FIG. 3B can include an outer skirt 104 formed of conventional material and attached to a radially-outer circumferential surface of the annular frame 102. The outer skirt 104 may also be coupled to the inner skirt 224, for example, by wrapping the outer skirt 104 around the inflow end 222 of the frame 102 into contact with the inner skirt 224, as shown in FIG. 3B, and overlapping portions of the skirts can be sutured together. Alternatively, the inner skirt 224 and the outer skirt 104 can be coupled together at the inflow end 222 in a manner similar to that illustrated in FIG. 3A. Alternatively, the inner skirt 224 can be wrapped around the inflow end 222 of the frame 102 into contact with the outer skirt 104, and overlapping portions of the skirts can be sutured together.

In some implementations, a prosthetic heart valve can include one or more protective covers on a radially-outer surface of the annular frame, for example, to protect commissures 112 of the valvular structure that extend radially outward of the respective inner skirts. For example, FIG. 3C shows an exemplary configuration of a prosthetic heart valve 240 where a protective cover 242a is disposed over commissures 112 on the radially-outer circumferential surface of the annular frame 102. The protective cover 242a comprises a hermetic layer that prevents, or at least reduces, tissue ingrowth into the portions of leaflet 106 forming commissures 112, for example, via native leaflet 40. In some implementations, the protective cover 242a can be provided only in regions where the commissure 112 are disposed. For example, the protective cover 242a can be integrated with the commissure 112, such as a coupling member that wraps around otherwise exposed surfaces of tabs of the leaflets. Optionally, the protective cover can be configured as an annular member that wraps around a radially-outer circumference of the annular frame 102, thereby also providing protective cover portions 242b in regions without commissures 112.

Alternatively, in some implementations, a prosthetic heart valve can mount the commissures 112 radially inward of the hermetic layer. In such configurations, a separate protective cover 242a may not be necessary. For example, FIG. 3D shows an exemplary configuration of a prosthetic heart valve 260 where the commissures 112 are coupled directly to the inner skirt 224, for example, using one or more sutures. Along the radial direction of the annular frame 102, the hermetic layer of the inner skirt 224 is provided between surrounding native tissue (e.g., leaflets 40) and the leaflets 106 of the prosthetic valve 100, thereby isolating the leaflets 106 from potential tissue ingrowth. FIG. 3E illustrates an exemplary attachment of a commissure assembly 112 to inner skirt 224, as viewed from a radially-inner side of the frame 102 of prosthetic heart valve 260. The inner skirt 224 may be attached to struts 262 of the frame 102 via one or more sutures 272. The interconnected struts 262 of the frame 102 can form an open cell 264 covered by the inner skirt 224 to which the commissure 112 can be attached. The tabs 268 of adjacent leaflets 106 can be splayed in opposite directions along the circumferential direction to form a T-shape and attached to the inner skirt 224 using one or more sutures 270. In this way, the commissure tab assemblies can be mounted to the frame without requiring separate commissure windows.

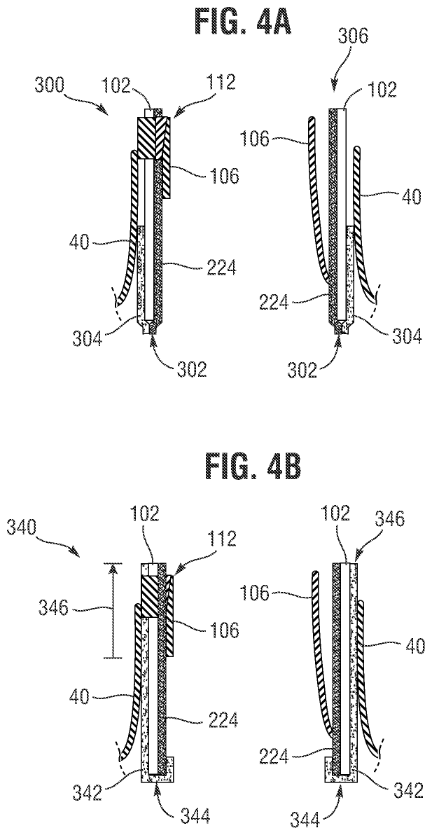

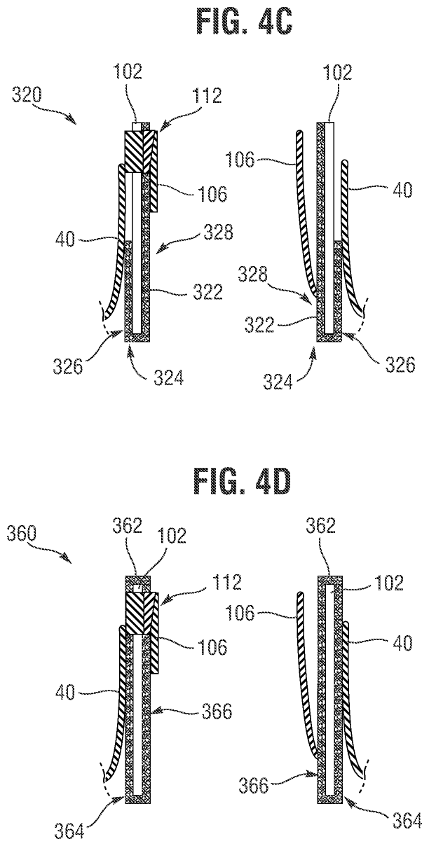

In some implementations, the inner skirt and the outer skirt can both comprise a hermetic layer. For example, FIG. 4A shows an exemplary configuration of a prosthetic heart valve 300 with inner skirt 224 and outer skirt 304 each comprising a hermetic layer. Similar to the configuration in FIG. 3B, the inner skirt 224 can extend from the inflow end 302 to the outflow end 306 of the valve, thereby providing a continuous barrier to tissue ingrowth over an entire radially-inner circumferential surface of annular frame 102. Alternatively, in some implementations, the inner skirt can extend along the axial direction of the frame 102 from the inflow end 302 to commissures 112, similar to the configuration illustrated in FIG. 3A. Leaflets 106 of the valvular structure of the prosthetic heart valve 200 can be coupled to the inner skirt 224 (e.g., via one or more sutures), and the commissures 112 can extend through respective openings in the inner skirt 224 to mount to the annular frame. In contrast to the configurations illustrated in FIGS. 3A-3D, the outer skirt 304 with a hermetic layer provides an additional barrier against ingrowth, thereby further inhibiting the native tissue from reaching the leaflets 106 of the prosthetic valve 300. The outer skirt 304 can be coupled to the frame 102 (e.g., via one or more sutures) and may optionally be coupled to the inner skirt 224, for example, via facing portions at the inflow end 302 (e.g., by one or more sutures, or by fusing, melting, or otherwise joining the hermetic layers of skirts 224, 304 together) or as described for other exemplary configurations above. Alternatively, or additionally, the inner skirt 224 can be fused or melted to the outer skirt 304 with the annular frame 102 therebetween in order to mount the skirts 224, 304 to the frame 102.

In some implementations, the outer skirt comprising the hermetic layer can be further extended to the outflow end of the prosthetic heart valve. For example, FIG. 4B shows an exemplary configuration of a prosthetic heart valve 340 where both the inner skirt 224 with hermetic layer and the outer skirt 342 with hermetic layer extend from the inflow end 344 to the outflow end 346, thereby providing a further continuous barrier to tis sue ingrowth over an entire radially-outer circumferential surface of annular frame 102. In order to mount the commissures 112 to the annular frame 102, one or more openings can be formed in the inner skirt 224 at locations corresponding to commissure windows of the annular frame 102. Corresponding openings may also be formed in the outer skirt 342. The commissures 112 can extend radially through the openings in the inner skirt 224, through the commissure windows, and through the openings in the outer skirt 342 for coupling (e.g., via one or more sutures) on a radially-outer side of the outer skirt 342.

Both the inner skirt 224 and the outer skirt 342 can be coupled to the frame 102, for example, using one or more sutures. The outer skirt 342 may also be coupled to the inner skirt 224, for example, by wrapping the outer skirt 342 around the inflow end 344 of the frame 102 into contact with the inner skirt 224, as shown in FIG. 4B, and overlapping portions of the skirts can be coupled together (e.g., by suturing, fusing, melting, or otherwise joining). Alternatively, the inner skirt 224 and the outer skirt 342 can be coupled together at the inflow end 344 in a manner similar to that illustrated in FIG. 4A. Alternatively, the inner skirt 224 can be wrapped around the inflow end 344 of the frame 102 into contact with the outer skirt 342, and overlapping portions of the skirts can be coupled together (e.g., by suturing, fusing, melting, or otherwise joining). Alternatively, or additionally, the inner skirt 224 can be fused or melted to the outer skirt 342 with the annular frame 102 therebetween in order to mount the skirts 224, 342 to the frame 102.

In some implementations, the inner skirt and the outer skirt can share the same hermetic layer. For example, FIG. 4C shows an exemplary configuration of a prosthetic heart valve 320 with a continuous skirt layer 322 comprising a hermetic layer. The continuous skirt layer 322 can be disposed over an entire radially-inner circumferential surface of the annular frame 102, thereby providing an inner skirt portion 328. Alternatively, in some implementations, the skirt layer 322 can extend over the radially-inner circumferential surface of the annular frame 102 from the inflow end 324 to commissures 112, similar to the configuration illustrated in FIG. 3A. A portion of the skirt layer 322 can be wrapped around the inflow end 324 and disposed over at least part (and optionally an entirety) of radially-outer circumferential surface of the annular frame 102, thereby providing an outer skirt portion 326. Similar to the above described configurations, leaflets 106 of the valvular structure of the prosthetic heart valve 320 can be coupled to the inner skirt portion 328 (e.g., via one or more sutures), and the commissures 112 can extend through respective openings in the inner skirt portion 328 to mount to the annular frame. In some implementations, the continuous skirt layer 322 can be secured to the annular frame using one or more sutures. Alternatively, or additionally, the inner skirt portion 328 can be fused or melted to the outer skirt portion 326 with the annular frame 102 therebetween in order to secure the continuous skirt layer 322 to the frame 102.

In some implementations, the inner and outer skirts sharing the same hermetic layer can further be extended to cover all surfaces of the frame of the prosthetic heart valve, thereby encapsulating the frame within the hermetic layer. For example, FIG. 4D shows an exemplary configuration of a prosthetic heart valve 360 having an annular frame 102 encapsulated within a hermetic layer 362. The encapsulating hermetic layer 362 can be disposed over all surfaces of the annular frame 102, thereby providing both an inner skirt portion 366 and an outer skirt portion 364 that acts as a barrier to tissue ingrowth. In some implementations, the encapsulating hermetic layer 362 is formed by placing separate hermetic sublayers on radially-inner and radially-outer circumferential surfaces of the annular frame 102, and then fusing, melting, or otherwise joining the hermetic sublayers together with the annular frame 102 therebetween. Alternatively, In some implementations, the encapsulating hermetic layer 362 if formed directly on the annular frame 102, for example, via dip coating, spray coating, electrospinning, or the like. Further details regarding materials and techniques for encapsulation and for attaching skirts to a prosthetic valve frame can be found in U.S. Pat. No. 8,945,209 and U.S. Patent Application Publication No. 2020/0155306, both of which are incorporated by reference in their entireties. In addition to providing the desired barrier to tissue ingrowth, the encapsulation process may also avoid, or at least reduce, time-consuming assembly associated with suturing skirts to the annular frame.

In some implementations, the hermetic layers of the inner skirt, the outer skirt, and/or the encapsulating layer can have sufficient strength and resiliency to avoid yielding or tearing (e.g., at suture holes), especially during transitions of the prosthetic valve between fully expanded and crimped configurations where the valve may be subjected to changes in longitudinal dimension of as much as 30%. Alternatively, or additionally, the inner skirt can include a scrim layer (e.g., a woven fabric or cloth, such as a PET fabric) in addition to the hermetic layer. The scrim layer can be disposed along an axial direction of the frame where the leaflets of the valvular structure are attached to the inner skirt. For example, in any of the illustrated examples of FIGS. 3A-4C, the inner skirt can have a scrim layer either between the hermetic layer and the annular frame or on a radially-inner side of the hermetic layer. Alternatively, or additionally, a scrim layer can be encapsulated with the frame by the hermetic layer. The scrim layer can be disposed along an axial direction of the frame where the leaflets of the valvular structure are attached to the encapsulating hermetic layer. For example, in the illustrated example of FIG. 4D, a scrim layer can be encapsulated with frame 102 by the encapsulating skirt layer 322. The scrim layer can improve the suture retention strength of the inner skirt or the encapsulating layer.

### Exemplary Prosthetic Heart Valves with Hermetic Layers

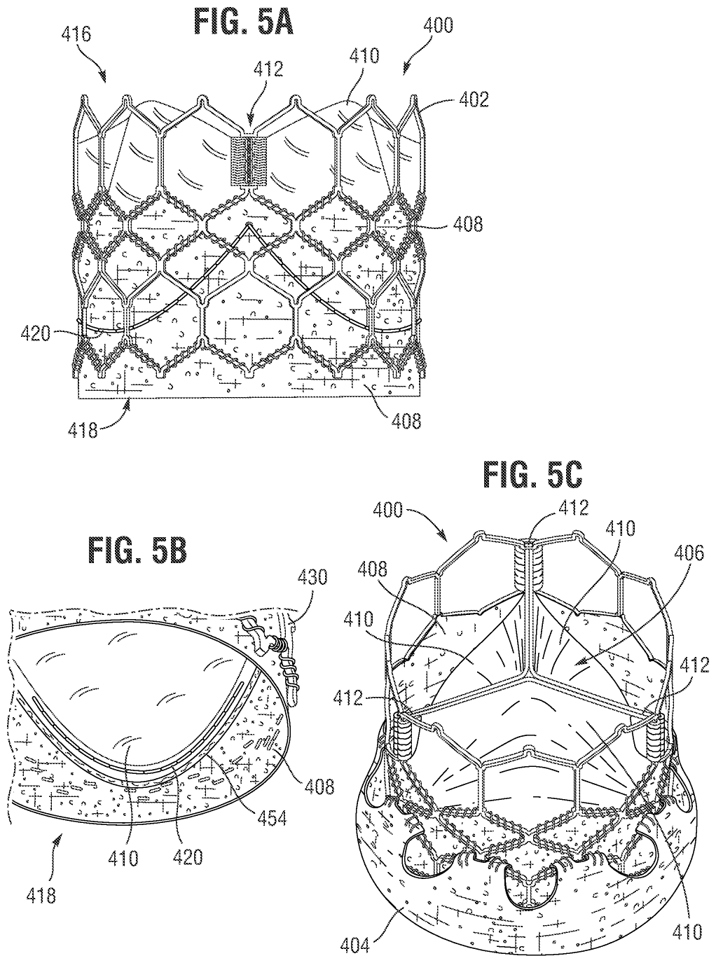

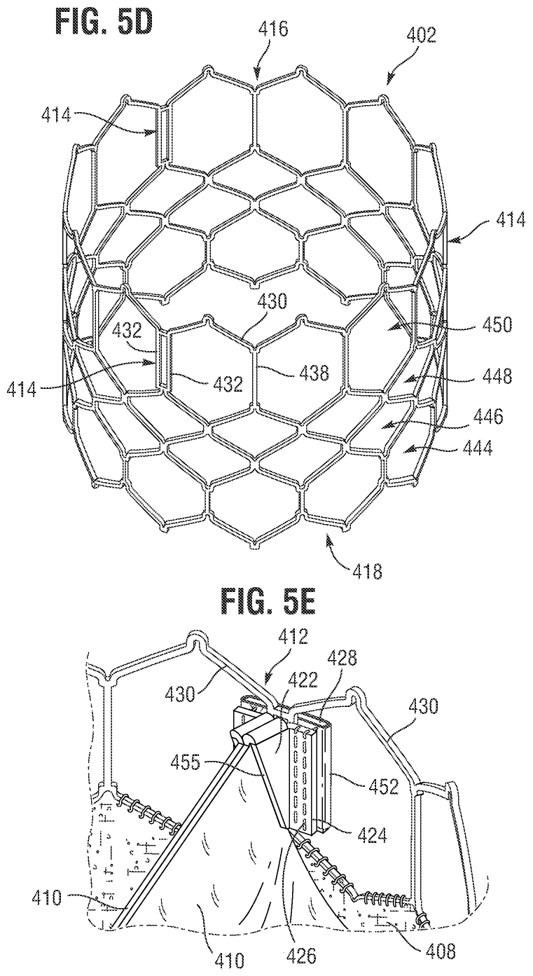

FIGS. 5A-5E illustrate various features of an exemplary prosthetic heart valve 400 that has an inner skirt comprising a hermetic layer. The prosthetic heart valve 400 can be crimped on or retained by an implant delivery apparatus in the radially-compressed configuration while the prosthetic heart valve is routed through the anatomy of a patient to the patient's heart, and then expanded to the radially-expanded configuration once the prosthetic heart valve reaches a desired implantation site within the heart. In particular examples, the prosthetic heart valve 400 can be implanted within the native aortic annulus, although it also can be implanted at other locations in the heart, including within the native mitral valve (e.g., mitral valve 16 in FIG. 1), the native pulmonary valve (e.g., pulmonary valve 30 in FIG. 1), or the native tricuspid valve (e.g., tricuspid valve 26 in FIG. 1). Prosthetic heart valves 400 can be implanted using any known delivery apparatus, for example, the delivery apparatus illustrated in FIG. 18.

The prosthetic heart valve 400 can include an annular stent or frame 402, which has a first axial end 416 and a second axial end 418. In the depicted example, the first axial end 416 can be an outflow end, and the second axial end 418 can be an inflow end. The outflow end 416 is the proximal-most end of the prosthetic valve when mounted on a delivery apparatus for delivering and implanting the prosthetic heart valve 400 within the native aortic valve using a transfemoral, retrograde delivery approach. In other implementations, the inflow end 418 can instead be the proximal-most end of the prosthetic valve when mounted on the delivery apparatus, depending on the particular native valve being replaced and the delivery technique that is used (e.g., trans-septal, transapical, etc.).

In some implementations, the frame 402, or components thereof (e.g., struts 430), can be made of any of various suitable plastically-expandable materials or self-expanding materials, as known in the art. Plastically-expandable materials that can be used to form the frame 402 can include, but are not limited to, stainless steel, biocompatible high-strength alloys (e.g., a cobalt-chromium or a nickel-cobalt-chromium alloys), polymers, or combinations thereof. In particular examples, frame 402 is made of a nickel-cobalt-chromium-molybdenum alloy, such as MP35N® alloy (SPS Technologies, Jenkintown, Pa.), which is equivalent to UNS R30035 alloy (covered by ASTM F562-13, Standard Specification for Wrought 35Cobalt-35Nickel-20Chromium-10Molybdenum Alloy for Surgical Implant Applications (UNS R30035), ASTM International, West Conshohocken, Pa., 2013, which is incorporated herein by reference). MP35N® alloy/UNS R30035 alloy comprises 35% nickel, 35% cobalt, 20% chromium, and 10% molybdenum, by weight. Self-expanding materials that can be used to form the frame 402 can include, but are not limited to, nickel titanium alloy (NiTi), such as nitinol.

When constructed of a plastically-expandable material, the frame 402 (and thus the prosthetic heart valve 400) can be crimped to the radially-compressed configuration on a delivery catheter and then expanded inside a patient by an inflatable balloon or equivalent expansion mechanism. Delivery apparatuses that have inflatable balloons for delivering prosthetic valves having plastically-expandable frames are disclosed in U.S. Patent Application Publication No. 2013/0030519, which is incorporated herein by reference. Alternatively, when constructed of a self-expanding material, the frame 402 (and thus the prosthetic heart valve 400) can be crimped to the radially-compressed configuration and restrained in the compressed configuration by insertion into a sheath or equivalent mechanism of a delivery catheter. Once advanced to the desired implantation site, the prosthetic heart valve can be advanced from the delivery sheath, thereby allowing the prosthetic heart valve to expand to its functional size. Further details of delivery apparatuses that can be used to deliver and implant self-expandable prosthetic valves (including any of the prosthetic valves disclosed herein when the frames are constructed of a self-expandable material such as nitinol) are disclosed in U.S. Patent Application Publication Nos. 2014/0343670 and 2010/0049313, which are incorporated herein by reference.

In some implementations, struts 430 of the frame 402 are pivotable or bendable relative to each other to permit radial expansion and contraction of the frame 102. For example, the frame 402 can be formed (e.g., via laser cutting, electroforming or physical vapor deposition) from a single piece of material (e.g., a metal tube). In other implementations, the frame 402 can be constructed by forming individual components (e.g., the struts and fasteners of the frame) and then mechanically assembling and connecting the individual components together. For example, instead of the strut structure illustrated in FIGS. 5A-5E, the frame can have individual diagonally-extending struts pivotably coupled to one another at one or more pivot joints along the length of each strut, as described in U.S. Patent Application Publication Nos. 2018/0153689, 2018/0344456, and 2019/0060057, all of which are incorporated herein by reference.

As best seen in FIG. 5D, the frame 402 can be formed with a plurality of circumferentially-spaced commissure windows 414. A valvular structure 406 can be coupled to the frame 402 at the commissure windows 414. For example, the valvular structure 406 can have a plurality of commissure assemblies 412, each corresponding to a respective one of the commissure windows 414 of the frame 402. In the illustrated example of FIG. 5A-5C, the valvular structure 406 comprises three leaflets 410 (e.g., a tricuspid structure), and the commissure windows 414 are equally spaced at 120° intervals (i.e., 0°, 120°, and 240°) along the circumference of the frame 402. However, other spacings and numbers of commissure windows 414 are also possible. For example, in some implementations, the valvular structure comprises two leaflets (e.g., a bicuspid structure), and the commissure windows are disposed on opposite sides of the frame (e.g., aligned on a same diameter of the frame).

As shown in FIGS. 5A-5C, the prosthetic heart valve 400 include an inner skirt 408, which comprises one or more hermetic layers. The inner skirt 408 can be mounted on an interior of the frame 402 (e.g., radially-inner circumferential wall formed by the lattice structure of the struts of the frame). The inner skirt 408 can span an entire circumference of the interior of the frame 402 and can extend along an axial direction of the frame 402, for example, from a location adjacent commissure windows 414 to a location at or just beyond the inflow end 418 of the frame 402. The inner skirt 408 can function as a sealing member to prevent, or at least reduce, perivalvular leakage (e.g., when the valve is placed at the implantation site) and as an attachment surface to anchor a portion of the leaflets 410 to the frame 402. For example, the cusp edge portions of the leaflets 410 can be attached to the inner skirt 408 via one or more sutures along suture line 420. The inner skirt can, in turn, be attached to selected struts of the frame 402, as illustrated in FIGS. 5A, 5C. In some implementations, the leaflets 410 can have a reinforcing member (e.g., fabric strip 454) along the inner surfaces of the cusp edge portions of the leaflets where the leaflets 410 are attached to the inner skirt 408, as shown in FIG. 5B.

As shown in FIG. 5C, the prosthetic heart valve 400 can also include an outer skirt 404. The outer skirt 404 can be mounted on an exterior of the frame 402 (e.g., radially-outer circumferential wall formed by the lattice structure of the struts of the frame). The outer skirt 404 can span an entire circumference of the exterior of the frame 402 and can extend along an axial direction of the frame, for example, from a location at or just beyond the inflow end 418 of the frame to a location about half of an axial height of the frame 402. The outer skirt 404 can function as a sealing member by sealing against the tissue of the native valve annulus and can help to reduce paravalvular leakage past the prosthetic heart valve 400.

The inner and outer skirts 408, 404 can be coupled to the frame 402 using sutures, adhesive, welding, and/or other means for attaching the skirts to the frame. Further details regarding frame construction, inner and outer skirts, techniques for assembling the leaflets to the inner skirt, and techniques for assembling the skirts on the frame, which may be employed in valve 400 or any other exemplary valve, are disclosed in U.S. Pat. No. 9,393,110, U.S. Patent Application Publication No. 2019/0192296, International Publication No. WO/2020/159783, and International Patent Application No. PCT/US2020/024559, each of which is incorporated herein by reference.

The outer skirt 404 can be formed of any of various suitable biocompatible materials, including any of various synthetic materials (e.g., polyethylene terephthalate (PET)) or natural tissue (e.g., pericardial tissue). Because the outer skirt 404 does not include a hermetic layer, the native tissue can grow into the outer skirt 404 and potentially invade the leaflets of the valvular structure. However, as discussed in detail above, the hermetic layer(s) of the inner skirt 408 can act as a barrier to further ingrowth of the native tissue onto the leaflets, thereby avoiding, or at least reducing the incidence of, pannus formation on leaflets 410 and reducing the concomitant risk of thrombosis.

The valvular structure 406 can be configured to allow blood flow through the frame 402 in only one direction, for example, to regulate the flow of blood through the prosthetic heart valve 400 from the inflow end 418 to the outflow end 416. The valvular structure 406 can include, for example, a leaflet assembly formed by a plurality of leaflets 410, each made of a flexible material. The leaflets 410 can transition between an open configuration, where blood flows through the valve 400 via a flow channel formed by the leaflets, and a closed configuration, where the leaflets occlude blood flow through the valve 400. The leaflets 410 can be made from in whole or part, biological materials, bio-compatible synthetic materials, or other such materials. Suitable biological materials can include, for example, bovine pericardium (or pericardium from other sources).

As shown in FIG. 5D, each commissure window 414 can be formed within or part of a lattice structure formed by axial struts 438 and angled struts 430. The struts 430, 438 of the frame can form circumferentially-extending rows of open cells 444, 446, 448, 450, with the row of cells 450 closest to the outflow end 416 have an open area greater than the other cells. In the illustrated example of FIG. 5D, each commissure window 414 can have a rectangular construction, with a central opening defined by a pair of side struts 432 (e.g., extending primarily along an axial direction of the frame 402) and a pair of cross-bars (e.g., extending primarily along a circumferential direction of the frame 402 at opposite ends of the struts 432). Other shapes and configurations for commissure window 414 are also possible. For example, instead of a rectangular opening, the commissure window can define an opening that is square, oval, square-oval, triangular, L-shaped, T-shaped, C-shaped, H-shaped, or any other shape.

FIG. 5E shows an exemplary approach for securing a commissure assembly 412 of the valvular structure to commissure window 414 of the annular frame 402. The commissure assembly 412 can include first tab portions 428 that extend through window 414 and are splayed along a circumferential direction of the frame 402 to form a T-shape. The first tab portions 428 can wrapped in, or at least partially covered by, a coupling member 452 (e.g., flexible cloth). The commissure assembly 412 can also include second tab portions 422 folded against the inner surface of the respective leaflet 410 and third tab portions 424, which extend along the circumferential direction of the frame 402 and face the first tab portions 428. The third tab portions 424 can coupled to the coupling member 452 and/or the respective first tab portions 428 via one or more sutures 426. The second tab portions 422 can form a multi-layer structure of leaflet material just inside the commissure window 414. The multi-layer structure can be more resistant to bending or articulating than the leaflet portions radially inward, thereby causing the leaflets 410 to articulate primarily at inner edges 455 of the second tab portions 422. The second tab portions 422 can thus help the leaflets to avoid contact with or damage from the frame 402 during normal operation of the valvular structure 406.

After all three commissure tab assemblies are secured to respective window frame 414, the lower edges of the leaflets 410 between the commissure assemblies 412 can be sutured to the inner skirt 408. For example, as shown in FIGS. 5A-5B, each leaflet 410 can be sutured to the inner skirt 408 along suture line 420 using, for example, Ethibond thread. The sutures can be in-and-out sutures extending through each leaflet 410, the skirt 408 and an optional reinforcing strip 454. In this manner, the lower edges of the leaflets 410 are secured to the frame 402 via the inner skirt 408. Further details regarding frame 402, valvular structure 406, and exemplary techniques for coupling the valvular structure to the frame are described in U.S. Pat. No. 9,393,110, which is incorporated herein by reference.

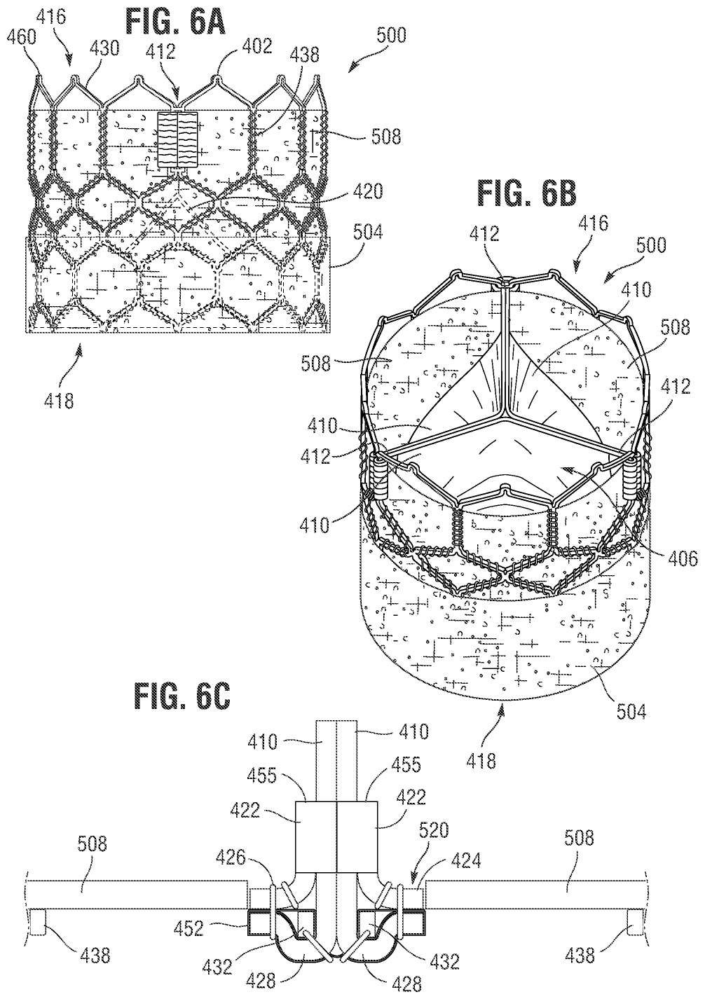

Although the above discussion of FIGS. 5A-5E specifies a particular configuration for prosthetic heart valve 400, other examples of a prosthetic heart valve, disclosed herein or otherwise, can include any of the innovations and variations discussed above with respect to FIGS. 3A-4D or later discussed below with respect to FIGS. 6A-21B.

FIGS. 6A-6C illustrate various features of another exemplary prosthetic heart valve 500. Similar to the prosthetic heart valve 400 illustrated in FIGS. 5A-5E, the exemplary prosthetic heart valve 500 of FIGS. 6A-6C has an annular frame 402, a valvular structure 406 comprising a plurality of leaflets 410, an inner skirt 508 comprising one or more hermetic layers, and an outer skirt 504. Similar to valve 400, the prosthetic heart valve 500 can be crimped on or retained by an implant delivery apparatus (e.g., the delivery apparatus illustrated in FIG. 18 or any other delivery apparatus) in the radially-compressed configuration while the prosthetic heart valve is routed through the anatomy of a patient to the patient's heart, and then expanded to the radially-expanded configuration once the prosthetic heart valve reaches a desired implantation site within the heart. In particular examples, the prosthetic heart valve 500 can be implanted within the native aortic annulus, although it also can be implanted at other locations in the heart, including within the native mitral valve (e.g., mitral valve 16 in FIG. 1), the native pulmonary valve (e.g., pulmonary valve 30 in FIG. 1), or the native tricuspid valve (e.g., tricuspid valve 26 in FIG. 1).

However, in contrast to the configuration illustrated in FIGS. 5A-5E, the inner skirt 508 of the valve 500 of FIGS. 6A-6C extends over the radially-inner circumferential surface of the annular frame 402 along the axial direction thereof from the inflow end 418 to the outflow end 416, and the outer skirt 504 also comprises one or more hermetic layers. The extension of the inner skirt 508 to the outflow end 416 can offer additional protection, for example, from native tissue ingrowth that may propagate via portions of the commissure assemblies 412, which portions are disposed on a radially-outer side of the annular frame and may contact surrounding native tissue (e.g., the native leaflets) of the patient. The outer skirt 504 can extend over the radially-outer circumferential surface of the frame 402 along the axial direction thereof from the inflow end 418 to an intermediate portion remote from the outflow end 416. In addition to the barrier offered by the hermetic layer(s) of the inner skirt 508, the provision of a hermetic layer on the exterior of the frame 402 (e.g., as part of outer skirt 504) offers an additional barrier to native tissue ingrowth, thereby further reducing the likelihood of pannus formation and the risk of thrombosis resulting therefrom.

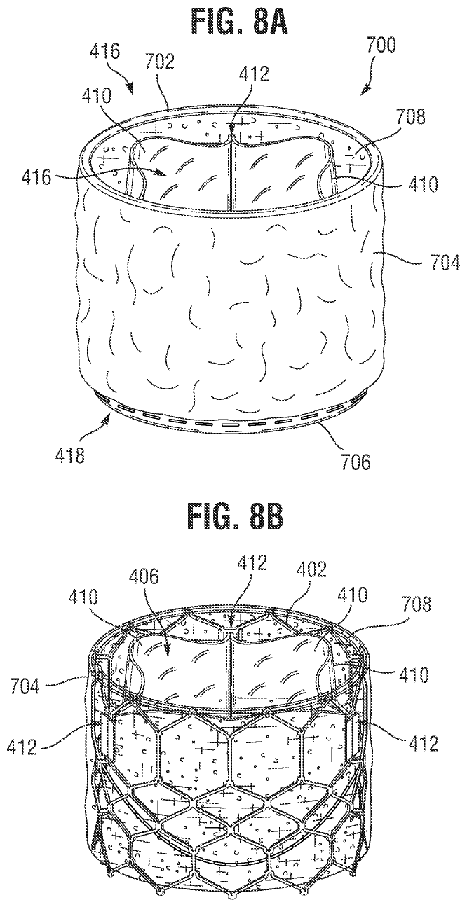

Although FIGS. 6A-6B show the inner skirt 508 as terminating at an end of axial struts 438, it is also possible for the inner skirt 508 to be extended along the axial direction of the frame 402 to the apices 460 formed by the angled struts 430 at the inflow end 418 of the frame 402 (for example, as shown in FIG. 8A). In either configuration, the one or more hermetic layers of the inner skirt 508 can provide a substantially continuous barrier to tissue ingrowth over an entire radially-inner circumferential surface of the annular frame 402. Although FIGS. 6A-6B show the outer skirt 504 as terminating at apices formed by angled struts 430 bordering open cells 444, it is also possible for the outer skirt to be extended along the axial direction of the frame 402 to a location closer to the outflow end 416, for example, to a location that covers suture line 420, to a location adjacent to and exposing commissure window 414, or to a location that covers commissure window 414 (for example, as shown in FIG. 8A).

The inner and outer skirts 508, 504 can be coupled to the frame 402 using sutures, adhesive, welding, and/or other means for attaching the skirts to the frame. For example, a portion of the outer skirt 504 can be wrapped around the inflow end 418 of the frame 402 into contact with the inner skirt 508, and the contacting portions of the skirts 504, 508 can be coupled together using one or more sutures. Alternatively or additionally, the inner and outer skirts 508, 504 can be coupled together with struts of the frame 402 captured therebetween, for example, by melting or fusing together portions of the skirts extending through open cells 444, 446. Similar to valve 400, the lower edges of leaflets 410 of valve 500 can be coupled to the inner skirt 508, for example, by one or more sutures along suture line 420. Further details regarding inner and outer skirts, techniques for assembling leaflets to the inner skirt, and techniques for assembling the skirts on the frame, which may be employed in valve 500 or any other exemplary valve, are disclosed in U.S. Pat. No. 9,393,110, U.S. Patent Application Publication Nos. 2019/0192296 and 2019/0365530, International Publication No. WO/2020/159783, and International Patent Application No. PCT/US2020/024559, each of which is incorporated herein by reference.

To allow the commissure assemblies 412 of the valvular structure 406 to pass through and mount to commissure windows 414, an opening 520 can be created in the inner skirt 508 at locations corresponding to the windows 414, for example, as illustrated in FIG. 6C. The opening 520 can have a dimension along the circumferential direction of the frame slightly larger than a width of the commissure assembly 412 attached to the window 414. The first tab portions 428 and the third tab portions 424 of the commissure assembly 412 can thus be coupled to each other, for example, using sutures 426, in a manner similar to that illustrated in FIG. 5E. Alternatively, the opening 520 can have a dimension along the circumferential direction of the frame smaller than a width of the commissure assembly 412. For example, the opening in the inner skirt 508 can be slightly smaller than the widths of the paired first tab portions 428 passing through window 414. In such a configuration, the sutures 426 for the first tab portions 428 and the third tab portions 424 may also pass through portions of the inner skirt 508 adjacent opening 520, or sutures can be provided to separately couple each of the first tab portions 428 and the third tab portions 424 to the portions of the inner skirt 508 adjacent opening 520.

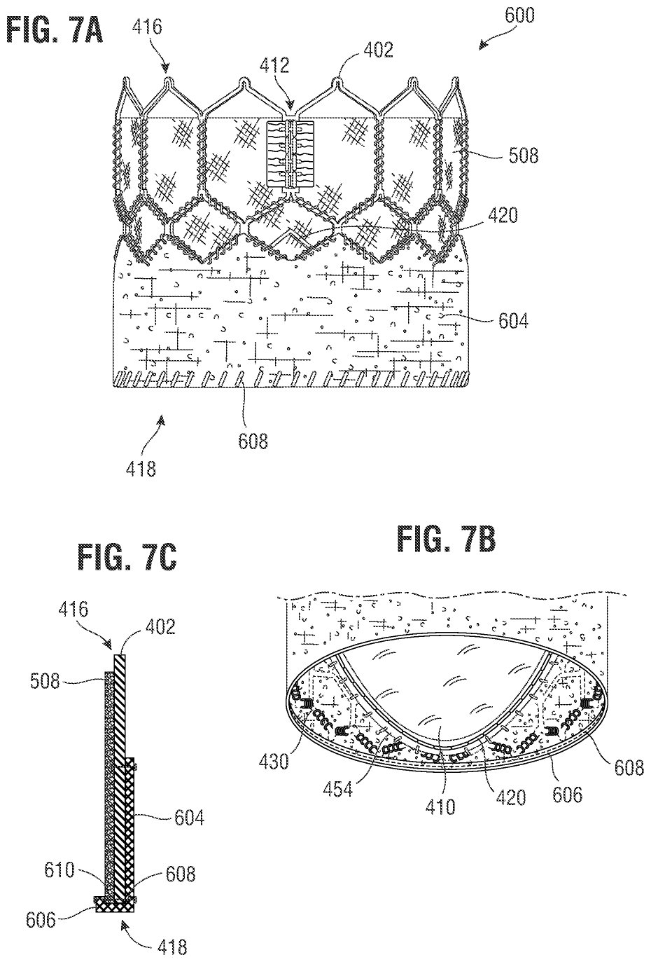

Although the above discussion of FIGS. 6A-6C specifies a particular configuration for prosthetic heart valve 500, other examples of a prosthetic heart valve, disclosed herein or otherwise, can include any of the innovations and variations discussed above with respect to FIGS. 3A-5E or later discussed below with respect to FIGS. 7A-21B.

FIGS. 7A-7C illustrate various features of another exemplary prosthetic heart valve 600. Similar to the prosthetic heart valve 500 illustrated in FIGS. 6A-6C, the exemplary prosthetic heart valve 600 of FIGS. 7A-7C has an annular frame 402, a valvular structure 406 comprising a plurality of leaflets 410, an inner skirt 508 comprising one or more hermetic layers, and an outer skirt 604. Similar to valve 500, the prosthetic heart valve 600 can be crimped on or retained by an implant delivery apparatus (e.g., the delivery apparatus illustrated in FIG. 18 or any other delivery apparatus) in the radially-compressed configuration while the prosthetic heart valve is routed through the anatomy of a patient to the patient's heart, and then expanded to the radially-expanded configuration once the prosthetic heart valve reaches a desired implantation site within the heart. In particular examples, the prosthetic heart valve 600 can be implanted within the native aortic annulus, although it also can be implanted at other locations in the heart, including within the native mitral valve (e.g., mitral valve 16 in FIG. 1), the native pulmonary valve (e.g., pulmonary valve 30 in FIG. 1), or the native tricuspid valve (e.g., tricuspid valve 26 in FIG. 1).

However, in contrast to the configuration illustrated in FIGS. 6A-6C, the outer skirt 604 can be formed of a non-hermetic material and can extend over the radially-outer circumferential surface of the frame 402 along the axial direction thereof from the inflow end 418 to apices formed by angled struts 430 bordering open cells 446. Alternatively, it is also possible for the outer skirt 604 to be extended along the axial direction of the frame 402 to a location closer to the outflow end 416, for example, to a location that covers suture line 420, to a location adjacent to and exposing commissure window 414, or to a location that covers commissure window 414. Because the outer skirt 604 does not include a hermetic layer, the native tissue can grow into the outer skirt 404 and potentially invade the leaflets of the valvular structure. However, as discussed in detail above, the hermetic layer(s) of the inner skirt 508 can act as a barrier to further ingrowth of the native tissue onto the leaflets, thereby avoiding, or at least reducing the incidence of, pannus formation on leaflets 410 and reducing the concomitant risk of thrombosis.

The outer skirt 604 can comprise at least one soft, plush surface oriented radially outward so as to cushion and seal against native tissues surrounding the valve 600. For example, the outer skirt 604 can be made from any of a variety of woven, knitted, or crocheted fabrics with the radially outer surface being a plush nap or pile of the fabric. Exemplary fabrics having a pile include velour, velvet, velveteen, corduroy, terrycloth, fleece, etc. Alternatively or additionally, the outer skirt 604 can comprise a non-woven fabric (e.g., felt) or fibers (e.g., non-woven cotton fibers). Alternatively, or additionally, the outer skirt 604 can be formed as or constructed from porous or spongey materials such as, for example, any of a variety of compliant polymeric foam materials, or woven fabrics, such as woven PET. In some implementations, the materials selected for the outer skirt 604 can contribute to improved compressibility and shape memory properties of the outer skirt. For example, a pile layer can be compliant such that is compresses under load (e.g., when in contact with native tissue, other implants, or the like) but otherwise returns to its original size and/or shape when the load is removed.

Various techniques and configurations can be used to secure the outer skirt 604 to the frame 402 and/or the inner skirt 508. For example, as shown in FIGS. 7B-7C, an first edge portion 606 of the outer skirt 604 can be wrapped around the inflow end 418 of the frame 402, and the first edge portion 606 of the outer skirt 604 can be attached to a contacting edge portion 610 of the inner skirt 508 and/or the frame 402, such as with one or more sutures 608 and/or an adhesive. In lieu of or in addition to sutures, the outer skirt 604 can be attached to the inner skirt 508, for example, by ultrasonic welding or any other coupling means. Alternatively, an edge portion of the inner skirt 508 can be wrapped around the inflow end 418 of the frame 402 into contact with a radially-outer surface of the outer skirt 604, and the contacting portions of the skirts 508, 604 attached together using one or more sutures, adhesive, welding, or any other coupling means. Further details regarding outer skirts and techniques for assembling the skirts on the frame, which may be employed in valve 600 or any other exemplary valve, are disclosed in U.S. Pat. No. 9,393,110, U.S. Patent Application Publication Nos. 2019/0192296 and 2019/0365530, International Publication No. WO/2020/159783, and International Patent Application No. PCT/US2020/024559, each of which is incorporated herein by reference.

Although the above discussion of FIGS. 7A-7C specifies a particular configuration for prosthetic heart valve 600, other examples of a prosthetic heart valve, disclosed herein or otherwise, can include any of the innovations and variations discussed above with respect to FIGS. 3A-6C or later discussed below with respect to FIGS. 8A-21B.

FIGS. 8A-8B illustrate features of another exemplary prosthetic heart valve 700. Similar to the prosthetic heart valve 500 illustrated in FIGS. 6A-6C, the exemplary prosthetic heart valve 700 of FIGS. 8A-8B has an annular frame 402, a valvular structure 406 comprising a plurality of leaflets 410, an inner skirt 708 comprising one or more hermetic layers, and an outer skirt 704 comprising one or more hermetic layers. Similar to valve 500, the prosthetic heart valve 700 can be crimped on or retained by an implant delivery apparatus (e.g., the delivery apparatus illustrated in FIG. 18 or any other delivery apparatus) in the radially-compressed configuration while the prosthetic heart valve is routed through the anatomy of a patient to the patient's heart, and then expanded to the radially-expanded configuration once the prosthetic heart valve reaches a desired implantation site within the heart. In particular examples, the prosthetic heart valve 700 can be implanted within the native mitral valve, although it also can be implanted at other locations in the heart, including within the native aortic valve (e.g., aortic valve 20 in FIG. 1), the native pulmonary valve (e.g., pulmonary valve 30 in FIG. 1), or the native tricuspid valve (e.g., tricuspid valve 26 in FIG. 1).

However, in contrast to the configuration illustrated in FIGS. 6A-6C, the inner skirt 708 of the valve 700 of FIGS. 8A-8B extends over an entirety of the radially-inner circumferential surface of the annular frame 402 from the inflow end 418 to the outflow end 416, and the outer skirt 704 extends over an entirety of the radially-outer circumferential surface of the annular frame 402 from the inflow end 418 to the outflow end. In some implementations, in addition to the one or more hermetic layers therein, the outer skirt 704 can further comprise a radially-outer layer that provides a soft, plush surface, is formed of a non-woven fabric or fibers, and/or is formed of a porous or spongey material. The outer skirt 704 may thus retain the function of cushioning and sealing against native tissues surrounding the valve (similar to that described above for outer skirt 604 in FIGS. 7A-7C), while at the same time offering a barrier against tissue ingrowth (similar to that described above for outer skirt 504 in FIGS. 6A-6C).

Additionally, or alternatively, the radially-outer layer can be designed and/or configured to prevent paravalvular leakage between the prosthetic valve 700 and the native valve (e.g., for when the valve frame is smaller in size than the corresponding native annulus into which it is implanted), to protect the native anatomy (e.g., to allow for smooth coaptation of the native leaflets against the valve), and/or to promote tissue ingrowth. Although such a radially-outer layer of the outer skirt 704 may allow for ingrowth, the hermetic layer(s) of the outer skirt 704 act as a barrier to further ingrowth. Moreover, the extension of the hermetic layers of the inner skirt 708 and outer skirt 704 over the entire interior and exterior surfaces of the frame can eliminate, or at least further reduce, potential avenues for tissue ingrowth, thereby further reducing the likelihood of pannus formation and the risk of thrombosis resulting therefrom.

In some examples, an inflow protective cap 706 can be formed from or disposed over the inner skirt 708 and/or the outer skirt 704 at the inflow end 418 of the valve 700, and/or an outflow protective cap 702 can be formed from or disposed over the inner skirt 708 and/or the outer skirt 704 at the outflow end 416 of the valve 700. When formed from the inner or outer skirts, the protective caps 702, 706 can be formed of a same material as one or more layers of the constituent skirt. For example, the protective cap 702 and/or 706 can be an extension of the hermetic layer of the outer skirt 704 that is wrapped over respective ends of the annular frame 402. Alternatively, when formed separately and disposed over the inner or outer skirts, the protective caps 702, 706 can comprise another hermetic layer, for example, to act as an additional barrier to tissue ingrowth. Alternatively, when formed separately and disposed over the inner or outer skirts, the protective caps 702, 706 can comprise biocompatible thermoplastic polymers such as PET, nylon, ePTFE, etc., or other suitable natural or synthetic fibers, or soft monolithic materials. In such configurations, the hermetic layers of the inner and outer skirts may act to otherwise isolate the protective caps 702, 706, thereby preventing any tissue ingrowth from propagating to the leaflets of the valvular structure 406.

The inner and outer skirts 708, 704 can be coupled to the frame 402 using sutures, adhesive, welding, and/or other means for attaching the skirts to the frame. For example, each of inner and outer skirts 708, 704 can be respectively sutured to facing struts of the frame 402. Alternatively, or additionally, the inner and outer skirts 708, 704 can be coupled together with struts of the frame 402 captured therebetween, for example, by suturing together or by melting or fusing together portions of the skirts extending through open cells 444-450 of the frame. Similar to valve 400, the lower edges of leaflets 410 of valve 500 can be coupled to the inner skirt 508, for example, by one or more sutures along suture line 420. Further details regarding construction of outer skirts, inflow/outflow protective portions, and techniques for assembling the skirts on the frame, which may be employed in valve 700 or any other exemplary valve, are disclosed in U.S. Pat. Nos. 9,393,110 and 10,195,025, U.S. Patent Application Publication Nos. 2018/0206982, 2019/0192296, 2019/0365530, 2019/0374337, and 2019/0046314, International Publication No. WO/2020/159783, and International Patent Application Nos. PCT/US2020/024559 and PCT/US2020/036577, each of which is incorporated herein by reference.

To allow commissure assemblies 412 of the valvular structure 406 to be coupled to commissure windows of the frame 402, openings can be formed in at least the inner skirt 708, and optionally in the outer skirt 704, at locations corresponding to windows 414. Alternatively, the outer skirt 704 can be attached to the valve frame 402 after mounting of the commissure assemblies 412 to the respective windows 414 of the frame 402, so as to avoid forming any openings for the commissure assemblies in the outer skirt 704. The commissure assemblies 412 can otherwise be attached to windows 414 in a manner similar to that described above for FIGS. 5E and 6C, and the leaflets can be sutured to the inner skirt 708 in a manner similar to that described above for FIGS. 5A-5B.

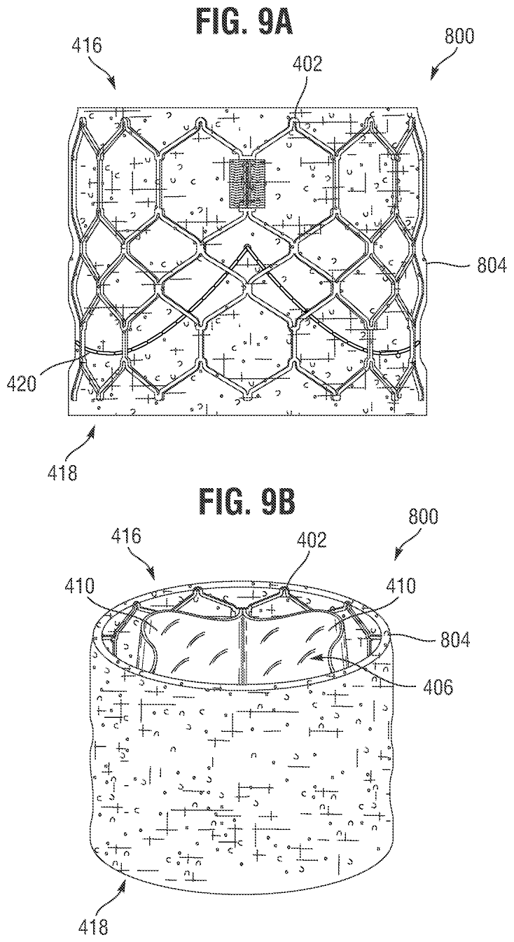

Although the above discussion of FIGS. 8A-8B specifies a particular configuration for prosthetic heart valve 700, other examples of a prosthetic heart valve, disclosed herein or otherwise, can include any of the innovations and variations discussed above with respect to FIGS. 3A-7C or later discussed below with respect to FIGS. 9A-21B.

FIGS. 9A-9B illustrate features of another exemplary prosthetic heart valve 800. Similar to the prosthetic heart valve 700 illustrated in FIGS. 8A-8B, the exemplary prosthetic heart valve 800 of FIGS. 9A-9B has an annular frame 402, a valvular structure 406 comprising a plurality of leaflets 410, and one or more hermetic layers. Similar to valve 700, the prosthetic heart valve 800 can be crimped on or retained by an implant delivery apparatus (e.g., the delivery apparatus illustrated in FIG. 18 or any other delivery apparatus) in the radially-compressed configuration while the prosthetic heart valve is routed through the anatomy of a patient to the patient's heart, and then expanded to the radially-expanded configuration once the prosthetic heart valve reaches a desired implantation site within the heart. In particular examples, the prosthetic heart valve 800 can be implanted within the native mitral valve, although it also can be implanted at other locations in the heart, including within the native aortic valve (e.g., aortic valve 20 in FIG. 1), the native pulmonary valve (e.g., pulmonary valve 30 in FIG. 1), or the native tricuspid valve (e.g., tricuspid valve 26 in FIG. 1).

However, in contrast to the configuration illustrated in FIGS. 8A-8B, the inner and outer skirts are replaced by an encapsulating layer 804 comprising one or more hermetic layers. The encapsulating layer 804 can fill the open cells 444-450 of the frame 402 and enclose the struts 430, 438 of the frame 402, such that the encapsulating layer 804 surrounds the entire annular frame 402 on all sides, thereby encapsulating the annular frame 402 within layer 804. The encapsulating layer 804 forms a radially-inner surface that acts as a hermetic inner skirt and a radially-outer surface that acts as a hermetic outer skirt. The encapsulating layer 804 thus provides both barriers both internal and external to the frame 402 against tissue ingrowth, thus reducing the likelihood of pannus formation.

The encapsulating layer 804 can be formed by pre-forming sublayers, disposing the sublayers on opposite sides of the frame 402, and then coupling the sublayers together to embed the annular frame 402 therein. For example, a first extruded sublayer can be disposed on a radially-inner circumferential surface of the annular frame 402 and a second extruded sublayer can be disposed on a radially-outer circumferential surface of the annular frame 402. The first and second sublayers can then be joined with the struts of the frame 402 therebetween, for example, by fusing, melting, welding, or the like. Alternatively, the encapsulating layer 804, or a portion thereof, can be formed directly on the frame, for example, by dip coating, spray coating, electrospinning, or the like. In addition to providing a barrier to tissue ingrowth, encapsulating layer 804 may also avoid, or at least reduce, time-consuming assembly associated with suturing skirts to the annular frame.

In some implementations, a separate outer skirt can be provided in addition to the encapsulating layer 804. For example, a separate outer skirt can be disposed on a radially-outer surface of the encapsulating layer 804 and coupled thereto (e.g., by sutures or any other coupling means). Similar to outer skirt 604 of FIGS. 7A-7C, the separate outer skirt coupled to the encapsulating layer 804 may comprise a radially-outer layer that provides a soft, plush surface, may be formed of a non-woven fabric or fibers, and/or may be formed of a porous or spongey material. The separate outer skirt can provide cushioning and sealing against native tissues surrounding the valve, while the encapsulating layer 804 acts as a barrier to prevent tissue ingrowth from reaching the leaflets 410 of the valvular structure 406.

In some implementations, a separate inner skirt or a scrim layer can be provided in addition to the encapsulating layer 804. For example, a separate inner skirt can be disposed on a radially-inner surface of the encapsulating layer 804 and coupled thereto (e.g., by sutures or any other coupling means). Alternatively, or additionally, a scrim layer (e.g., woven fabric or cloth) may be disposed on a radially-inner surface of the annular frame 402 prior to encapsulation, and the encapsulating layer 804 may enclose both the annular frame 402 and the scrim layer. The separate inner skirt and/or scrim layer can increase suture retention strength for attachment of the leaflets 410 thereto, while the encapsulating layer 804 acts as a barrier to prevent tissue ingrowth from reaching the leaflets 410 of the valvular structure 406.

To allow the commissure assemblies 412 of the valvular structure 406 to pass through and mount to commissure windows 414, openings can be created in the encapsulating layer 804, at locations corresponding to the windows 414. In some implementations, the openings are created after the encapsulation layer 804 is formed on the annular frame 402, for example, by cutting away the layer 804 in a region surrounding window 414 and/or by piercing through the layer 804 covering the opening of the window 414. Alternatively, In some implementations, the openings can be created during formation of the encapsulation layer 804 on the annular frame 402, for example, by covering the windows 414 during the encapsulation process, inserting a temporary sacrificial member within the window openings during the encapsulation process, or otherwise preventing material from forming over and obstructing windows 414 during the encapsulation process. Alternatively, in some implementations, the openings can be created in one or more of the sublayers used to form the encapsulation layer 804 prior to encapsulation of the annular frame 402. The commissure assemblies 412 can otherwise be attached to windows 414 in a manner similar to that described above for FIGS. 5E and 6C, and the leaflets can be sutured to the encapsulating layer 804 in a manner similar to that described above for FIGS. 5A-5B.

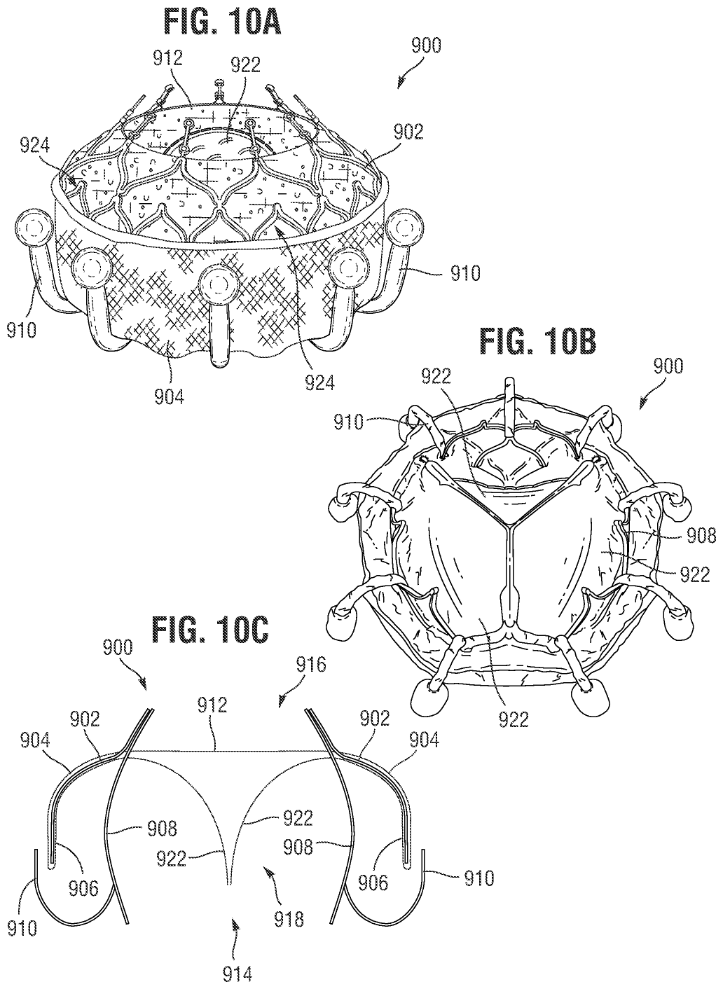

Further details regarding replacement of inner or outer skirts using an encapsulating layer, materials for encapsulating layers, and techniques for encapsulation and for attaching leaflets to the encapsulating layers, which may be employed in valve 800 or any other exemplary valve, can be found in U.S. Pat. No. 8,945,209 and U.S. Patent Application Publication No. 2020/0155306, both of which are incorporated by reference in their entireties. Although the above discussion of FIGS. 9A-9B specifies a particular configuration for prosthetic heart valve 800, other examples of a prosthetic heart valve, disclosed herein or otherwise, can include any of the innovations and variations discussed above with respect to FIGS. 3A-8B or later discussed below with respect to FIGS. 10A-21B.

FIGS. 10A-10C illustrate various features of another exemplary prosthetic heart valve 900 having a dual frame construction and primarily intended for implantation in the native mitral valve or native tricuspid valve. Referring initially to FIG. 10C, a cross-sectional view of the prosthetic heart valve 900 in an expanded configuration is shown. The prosthetic valve 900 can include an inner frame 908, an outer frame 902, a valvular structure 918 comprised of a plurality of leaflets 922, and one or more skirts, such as an outer skirt 904 and an inner skirt 906. The inner frame can have a generally bulbous shape such that the diameters of regions near the inflow end 916 and the outflow end 914 are less than the diameters of an intermediate region between the inflow and outflow end regions. The outer frame 902 can be coupled to the inner frame 908 using any suitable fastener and/or technique. Alternatively, the inner and outer frames 908, 902 can be formed as a unitary or monolithic structure.

The valvular structure 918 can include a plurality of leaflets 922, for example, three leaflets, which are joined at commissures. The valvular structure 918 can also include one or more intermediate components 912 (which can be made of fabric), which are positioned between a portion of, or the entirety of, the leaflets 922 and the inner frame 908. Accordingly, at least a portion of each leaflet 922 can be coupled to the inner frame 908 via the intermediate component 912, such that part of, or the entirety of, the portion of each leaflet 922 at the commissures and/or a cusp edge of the leaflet 922 is not directly coupled to the inner frame 908. Rather, the leaflets 922 may be considered to be indirectly coupled to, or floating within, the inner frame 908. For example, part of, or an entirety of, the portion of each leaflet 922 proximate the commissures and/or the cusp edge of the leaflet 922 can be spaced radially inward from an inner surface of the inner frame. Such a configuration may allow greater flexibility in selection of geometry for the valve frame (e.g., non-cylindrical frames to better fit the native annulus) and/or sizes for the valve frame (e.g., frames having a diameter greater than that of the valvular structure).

The outer skirt 904 of prosthetic heart valve 900 can be coupled to the inner frame 908 and/or the outer frame 902. In the illustrated example of FIG. 10C, the outer skirt 904 is positioned around and secured to an exterior of the outer frame 902. The outer skirt 904 can also be secured to a portion of the valvular structure 918, for example, at a portion of the intermediate components 912 near inflow end 916. The inner skirt 906 of the prosthetic heart valve 900 can be coupled to the valvular structure 918 and the outer skirt 904. In the illustrated example of FIG. 10C, a first end of inner skirt 906 is coupled to the valvular structure 918 along portions thereof proximate to the inner frame 908, and a second end of the inner skirt 906 is coupled to a lower region of the outer skirt 904. Accordingly, a smooth surface can be formed under each of the leaflets 922, which may beneficially enhance hemodynamics while reducing areas of stagnation.

The outer frame 902 can include a plurality of struts, with at least some of the struts forming respective cells 924. Any number and configuration of struts can be used, such as rings of undulating struts forming ellipses, ovals, rounded polygons, teardrops, chevrons, diamonds, curves, or any other shape. The outer frame 902 can be used to engage with a native valve annulus, native valve leaflets, or any other tissue or body cavity, while spacing an inflow end 916 of the valve 900 from the heart or vessel wall. The inner frame 908 can include one or more anchoring projections 910, which can be configured to contact or engage a native mitral valve annulus on a ventricular side, tissue beyond the native valve annulus on a ventricular side, native leaflets on a ventricular side, and/or other tissue at or around the implantation location during one or more phases of the cardiac cycle, such as systole and/or diastole. In particular examples, the anchoring projections 910 can extend behind the native leaflets (e.g., the native mitral valve leaflets or native tricuspid valve leaflets). When positioned within the native mitral valve (or tricuspid valve), the anchoring projections 910 can beneficially eliminate, inhibit, or limit movement of the implanted prosthetic valve 900, for example, when subjected to forces directed from the outflow end 914 toward the inflow end 916 during systole.

Any of the intermediate components 912, the inner skirt 906, and the outer skirt 904 can comprise one or more hermetic layers. For example, at least the intermediate components 912 comprise one or more hermetic layers, while the inner skirt 906 and outer skirt 904 are formed of an impermeable but porous material. In such a configuration, the native tissue can grow into the outer and inner skirts, but further ingrowth of the native tissue onto the leaflets 922 is blocked by the hermetic layer of the intermediate components 912, to which the leaflets 922 are coupled. Alternatively, at least the inner skirt 906 and the intermediate components 912 comprise one or more hermetic layers, thereby providing multiple barriers against native tissue ingrowth. In yet another alternative, each of the intermediate components 912, the inner skirt, 906, and the outer skirt 904 comprises one or more hermetic layers, thereby further insulating the leaflets 922 from potential ingrowth of native tissue and potential pannus formation. In some implementations, other components of prosthetic valve 900 in contact with the native tissue may also include one or more hermetic layers, such as part of, or the entirety of, anchoring projections 910. In some implementations, the hermetic layers of the intermediate components 912, the inner skirt 906, outer skirt 904, and/or anchoring projections 910 are separately formed (e.g., extrusion, casting, etc.) and subsequently coupled (e.g., by suturing, welding, fusing, etc.) to the inner frame 908 or outer frame 902. Alternatively, or additionally, the hermetic layers of the intermediate components 912, the inner skirt 906, outer skirt 904, and/or anchoring projections 910 can be formed directly on the inner frame 908 or outer frame 902, for example, by coating or encapsulating (e.g., electrospinning, dip coating, or spray coating).

In particular examples, the prosthetic heart valve 900 can be implanted within the native mitral valve, although it also can be implanted at other locations in the heart, including within the native aortic valve (e.g., aortic valve 20 in FIG. 1), the native pulmonary valve (e.g., pulmonary valve 30 in FIG. 1), or the native tricuspid valve (e.g., tricuspid valve 26 in FIG. 1). Further details regarding construction and operation of the inner and outer frames, configuration of intermediate portions and inner and outer skirts, implantation, and delivery systems for implantation, which may be employed with valve 900 or any other exemplary valve, are disclosed in U.S. Pat. No. 10,350,062 and U.S. Patent Application Publication Nos. 2018/0055629 and 2019/0262129, each of which is incorporated herein by reference.

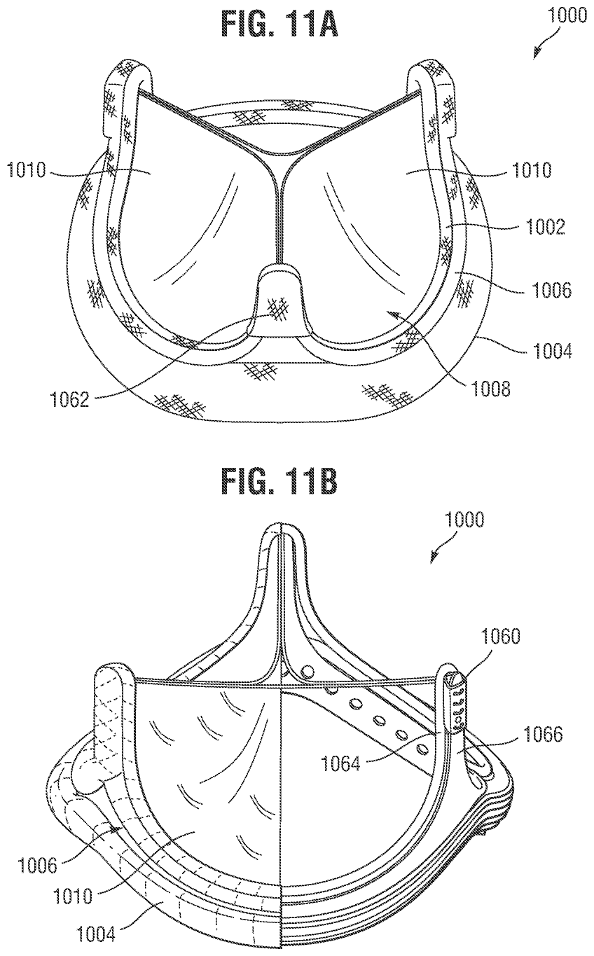

Although the above discussion of FIGS. 10A-10C specifies a particular configuration for prosthetic heart valve 900, other examples of a prosthetic heart valve, disclosed herein or otherwise, can include any of the innovations and variations discussed above with respect to FIGS. 3A-9B or later discussed below with respect to FIGS. 11A-21B.

FIGS. 11A-11B illustrate various features of another exemplary prosthetic heart valve, in particular, a surgical valve 1000 having components comprising one or more hermetic layers. The surgical valve 1000 generally comprises a wireform assembly 1002, a sewing ring assembly 1004, a stent assembly 1006, and a valvular structure 1008. The valvular structure 1008 can comprise three leaflets 1010 in a tricuspid arrangement. The wireform assembly 1002 can comprise a wireform, a cloth cover surrounding the wireform, and one or more encapsulating layers surrounding the cloth cover. The encapsulating layer comprises one or more hermetic layers, thereby providing a barrier to tissue ingrowth into the wireform assembly 1002. The wireform can be formed from one or more pieces of wire, such as stainless steel or an alloy of Co—Cr—Ni such as Elgiloy (e.g., 39-41% cobalt, 19-21% chromium, 14-16% nickel, 11.3-20.5% iron, 6-8% molybdenum, and 1.5-2.5% manganese). The cloth cover can be formed of any biocompatible fabric, such as, for example, PET. The cloth cover comprises an elongated strip of material having opposing ends that are brought together to form a butt joint. Opposing longitudinal edges of the cloth cover can then be wrapped around the wireform and coupled together (e.g., via stitching). Alternatively, the encapsulating layer formed around the cloth cover can be used to secure the cloth cover to the wireform instead of separate stitching together edges of the cloth cover.

The sewing ring assembly 1004 can comprise a sewing ring insert, a second cloth cover (e.g., PET) surrounding the insert, and one or more second encapsulating layers surrounding the second cloth cover. The second encapsulating layer comprises one or more hermetic layers, thereby providing a barrier to tissue ingrowth into the sewing ring assembly 1004. The sewing ring insert can have a conventional construction and can be made of a suture permeable material for suturing the valve to a native annulus. For example, the sewing ring insert can be formed of a silicone-based material, although other suture-permeable materials can be used. Similar to the wireform assembly 1002, the second encapsulating layer formed around the second cloth cover can also be used to secure the second cloth cover to the sewing ring insert instead of separate stitching together edges of the cloth cover.

The stent assembly 1006 can comprise an inner support and an outer band disposed around the inner support. The inner support can comprise cups portions extending between upstanding commissure portions. The outer band can be shaped to conform to the curvature of the cusp portions of the inner support. For example, the inner support can be formed of a polymeric material, such as polyester, and the outer band can be formed of a relatively rigid metal, such as a Co—Cr—Ni alloy (e.g., Elgiloy) or stainless steel. A third cloth cover can completely cover the inner support and the outer band. One or more third encapsulating layers can surround the third cloth cover. The third encapsulating layer comprises one or more hermetic layers, thereby providing a barrier to tissue ingrowth into the stent assembly 1006. Similar to the wireform assembly 1002, the third encapsulating layer formed around the third cloth cover can also be used to secure the third cloth cover to the inner support and outer band instead of separate stitching together edges of the cloth cover.

Once the wireform assembly 1002, sewing ring assembly 1004, and stent assembly 1006 are formed, these components can be assembled together with leaflets 1010 to form the assembled valve 1000. For example, three leaflets 1010 can be positioned with the wireform assembly 1002. Each leaflet 1010 can include two tabs positioned on opposing ends of the leaflet. Each respective tab can be aligned with a tab of an adjacent leaflet to form a commissure assembly 1060. The lower edge of each leaflet 1010 extending between the tabs can be sutured to the wireform assembly 1002, for example, to the encapsulating layer and/or cloth covering thereof. Each commissure assembly 1060 can be inserted between adjacent upright extensions 1064 and wrapped around a respective commissure post 1066 of the stent assembly 1006. The tabs of the commissure assembly 1060 can be sutured or otherwise coupled to each other and/or to the commissure post 1066.

The wireform assembly 1002 can be then be secured to an upper inner portion of the stent assembly 1006 and the sewing ring assembly 1004 can be secured to a lower outer portion of the stent assembly 1006. The stent assembly 1006 can mate with or engage a corresponding contour of the wireform assembly 1002. Thus, the commissure posts 1066 and the cusp portions extending between the commissure posts can be sized and shaped so as to correspond to the curvature of the wireform assembly. The wireform assembly 1002 can be secured to the stent assembly 1006 via sutures extending through the cloth covering of the wireform assembly and the apertures in the inner support and outer band of the stent assembly 1006. The sewing ring assembly 1004 can be secured to the stent assembly 1006 via sutures extending through the sewing ring assembly and apertures in the inner support and outer band of the stent assembly. Protective covers 1062 can be positioned over the exposed portions of commissure assembly 1060 (e.g., tabs of the leaflets 1010), and secured in place with sutures. In some implementations, the covers 1062 also comprise one or more hermetic layers.

As with the other examples discussed above, the provision of the encapsulating hermetic layers in the wireform assembly 1002, sewing ring assembly 1004, and stent assembly 1006 provides a barrier against tissue ingrowth reaching the leaflets 1010 of the valvular structure 1008. As a result, the incidence of pannus formation on the leaflets 1010 can be reduced, and the risk of thrombosis resulting from such pannus can be mitigated. In some implementations, some of the assemblies or parts thereof may be designed to encourage tissue ingrowth, while the remaining assemblies or parts include a hermetic layer to prevent the tissue ingrowth from reaching the leaflets 1010. For example, the sewing ring assembly 1004 may be constructed without a hermetic layer to allow for tissue ingrowth, while the wireform assembly 1002 and the stent assembly 1006, which are otherwise directly coupled to parts of the leaflets 1010, can have respective hermetic layers that inhibit further propagation of the tissue from the sewing ring assembly 1004 to the leaflets 1010. Further details regarding construction of the various assemblies and encapsulation layers thereof, which may be employed with valve 1000 or any other exemplary valve, are disclosed in U.S. Patent Application Publication No. 2020/0155306, which is incorporated herein by reference.

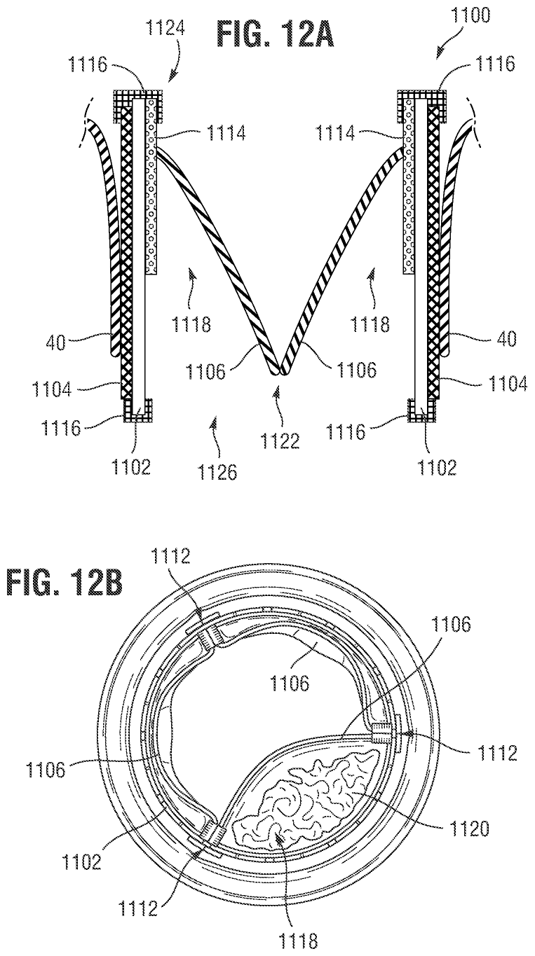

Although the above discussion of FIGS. 11A-11B specifies a particular configuration for prosthetic heart valve 1000, other examples of a prosthetic heart valve, disclosed herein or otherwise, can include any of the innovations and variations discussed above with respect to FIGS. 3A-10B or later discussed below with respect to FIGS. 12A-21B. Moreover, while specific examples of prosthetic heart valves are discussed above, the provision of one or more hermetic layers to prevent propagation of tissue ingrowth onto leaflets is applicable to a wide variety of prosthetic valves. For example, the existing inner or outer skirts in the prosthetic heart valves disclosed in any of U.S. Pat. Nos. 6,730,118, 7,101,396, 7,393,360, 7,510,575, 7,993,394, 8,652,202, 8,992,608, 9,339,382, and 10,603,165, U.S. Patent Application Publication Nos. 2018/0325665, 2018/0344456, and 2019/0060057, and International Publication No. WO/2020/081893, all of which are incorporated by reference herein, can be replaced with, or at least supplemented by, one or more hermetic layers. Alternatively or additionally, any of the prosthetic heart valves disclosed in any of U.S. Pat. Nos. 6,730,118, 7,101,396, 7,393,360, 7,510,575, 7,993,394, 8,652,202, 8,992,608, 9,339,382, and 10,603,165, U.S. Patent Application Publication Nos. 2018/0325665, 2018/0344456, and 2019/0060057, and International Publication No. WO/2020/081893, can be modified in accordance with the teachings of the present disclosure to include one or more hermetic layers between the surrounding native tissue and attachment points for the leaflets of the prosthetic heart valve, for example, between at least a radially-inner surface of the valve frame and the leaflets.

Low Opening Pressure Valvular Structures

As discussed above with respect to FIG. 1, prosthetic heart valves implanted at the aortic position (e.g., within the native aortic valve 20) can normally experience relatively-high pressure gradients (e.g., driving pressure of about 125 mmHg). In contrast, prosthetic heart valves implanted at the pulmonary position (e.g., within the native pulmonary valve 30), the tricuspid position (e.g., within the native tricuspid valve 26), the mitral position (e.g., within the native mitral valve 16), or blood vessels leading to heart chambers (e.g., within the inferior vena cava 36 or superior vena cava 34) can normally experience relatively-low pressure gradients (e.g., driving pressure of about 30 mmHg or less).

For examples, FIGS. 12A-12B illustrate an exemplary prosthetic heart valve 1100 implanted between leaflets 40 of the native mitral valve. The prosthetic heart valve 1100 can have a frame 1102, an inner skirt 1114, an outer skirt 1104, and a valvular structure 1122 comprising a plurality of leaflets 1106. The leaflets 1106 can be coupled to the frame via commissure assemblies 1112 mounted to respective windows of the frame 1102 and via cusp edge portions mounted to the inner skirt 1114 (e.g., via one or more sutures), as previously described. One or more protective cap or covering layers 1116 can be disposed over struts of the frame 1102 at its inflow end 1124 and/or at its outflow end 1126, as shown in FIG. 12A. The prosthetic heart valve 1100 can thus have a configuration similar to that described in any of U.S. Patent Application Publication Nos. 2018/0206982, 2019/0192296, and 2019/0374337, incorporated by reference above, and further details can be found therein. Alternatively or additionally, the prosthetic heart valve 110 can have a configuration similar to that described in any of U.S. Pat. No. 10,350,062 and U.S. Patent Application Publication Nos. 2018/0055629 and 2019/0262129, each of which is incorporated herein by reference.

Since motion of the leaflets of the valvular structure is driven by the pressure across the valve, such low-pressure implant locations may result in abnormal motion of the leaflets, for example, stasis and/or delayed or sluggish opening of the valvular structure, which may contribute to chronic thrombosis and/or thickening of the leaflets. Under low-pressure flow conditions associated with such implant locations, the neo-sinus region 1118, formed between the leaflets 1106 and the annular frame 1102, may experience inadequate washout, thereby making the valvular structure 1122 more susceptible to formation of thrombi 1120, as illustrated in FIG. 12B.

Accordingly, in some implementations, the valvular structure of the prosthetic heart valve can be modified to facilitate transition of the leaflets between open and closed configurations and to encourage adequate wash-out of the neo-sinus region when implanted at a low-pressure position. In some implementations, the shape of the leaflets can be modified, for example, the shape and arrangement of the curved cusp edge, which is coupled to the valve frame via the inner skirt, with respect to the leaflet tabs, which form commissure assemblies that attach to respective windows of the valve frame. In some implementations, the suture line used to attach the leaflet cusp edge to the inner skirt is extended to a location substantially at or adjacent to the mounted commissure assemblies. In some implementations, the shape of the curved cusp edge can be made more shallow than conventional leaflet designs, for example, to encourage washout of the neo-sinus region.

For example, FIGS. 13A-13C illustrate aspects of an exemplary prosthetic heart valve with modified valvular structure comprising a plurality of leaflets 1200. The leaflets 1200 of the valvular structure can be made from in whole or part, biological materials, bio-compatible synthetic materials, or other such materials. Suitable biological materials can include, for example, bovine pericardium (or pericardium from other sources). As shown in FIG. 13A, at a free edge (or coaptation edge) of the leaflet 1200 (the upper edge in the figure), a first portion 1204 can extend between a pair of tabs 1208 (also referred to herein as leaflet tabs or commissure tabs) on opposite ends of leaflet 1200 with respect to centerline 1202 of the first portion 1204. As used here, “upper” and “lower” may be relative to a central longitudinal axis of the prosthetic heart valve when the valvular structure is installed and coupled to frame 1302, with upper being closer to an outflow end of the valve and lower being closer to an inflow end of the valve. The first portion 1204 defines a first edge 1206 (also referred to as upper edge) that can extend between the tabs 1208. Each tab 1208 can have at least an outer edge 1210 and a base edge 1212. In some implementations, the outer edges 1210 of the tabs 1208 are substantially parallel to each other, e.g., both edges 1210 extend along direction 1226. In some implementations, outer edges 1210 (and direction 1226) can also be parallel to the centerline 1202 of the first portion 1204.

At the lower edge of the leaflet 1200, a second portion 1214 (also referred to herein as a cusp edge portion) is provided on a side of the first portion opposite the first edge 1206. The second portion 1214 can define a cusp edge 1216 that extends between base ends 1212 of tabs 1208 and that is curved along its entire length (or substantially along the entire length) between the base edges 1212 of the tabs 1208. The curvature of the cusp edge 1216 may thus continue to locations 1222 substantially at or adjacent to respective base edges 1212 of tabs 1208, as illustrated in FIG. 13A. The curvature of the cusp edge 1216 may also be such that a tangent 1224 of the cusp edge 1216 at location 1222 is substantially parallel to the outer edges 1210 of the tabs 1208 (e.g., parallel to direction 1226). Conversely, the curvature of the cusp edge 1216 may be such that a tangent at any other location besides 1222 along the cusp edge is not parallel to the outer edges 1210 of the tabs 1208 (e.g., the tangent crosses direction 1226). The curvature of the cusp edge 1216 can define an apex 1230, which, in some implementations, can coincide with the centerline 1202 of the first portion 1204

In some implementations, the second portion 1214 may have the shape of a half-ellipse or semi-ellipse, with the cusp edge 1216 following a curvature defined by the half-elliptical or semi-elliptical shape. The half-elliptical or semi-elliptical shape can be on a major axis 1228 (on which foci 1218 of the ellipse are disposed) that is substantially parallel to the base edges 1212 of the tabs 1208 and/or substantially perpendicular to the centerline 1202 of the first portion 1204. In some implementations, the major axis 1228 is substantially coincident with the base edges 1212, as illustrated in FIG. 13A.

Tabs 1208 of adjacent leaflets 1200 can be paired together to form commissure assemblies 1304 that are then coupled (directly or indirectly) to respective commissure windows 1312 of the frame 1302, as illustrated in FIG. 13B. For example, the coupling of the commissure assemblies to the frame window can be a manner similar to that described above with respect to FIG. 5E, or as otherwise described in U.S. Pat. No. 9,393,110, incorporated by reference above. The outer edges 1210 of the tabs 1208 are thus external to the valve frame 1302 and may be positioned to extend along a direction substantially parallel to an axial direction of the frame 1302. In some implementations, the suture line 1220 used to attach the second portion 1214 of each leaflet 1200 to the inner skirt 1314 can be extended to a location 1324 substantially at or adjacent to the mounted commissure assemblies 1304 (e.g., the base edges 1212 of the corresponding tabs 1208). In some implementations, due to the presence of struts 1308 of the frame 1302 and/or the cross-bar 1306 of the window 1312, location 1324 may be spaced away from the base edge 1212 along the axial direction of the frame 1302, for example, to allow access during suturing of the leaflets 1200 to the inner skirt 1314 and/or to avoid a region where the inner skirt 1314 is otherwise sutured to struts 1308 of the frame 1302. Nevertheless, the location 1324 of the suture line is preferably as close as possible to the base edge 1212.

The combination of the half-elliptical or semi-elliptical shape of the second portion 1214, the parallel arrangement of the outer edges 1210 of the tabs 1208 and the tangents 1224 of the cusp edge 1216, and the continuous suture line 1220 extending to (or as close as possible to) the base edges 1212 of the tabs 1208 can allow the valvular structure formed by the leaflets to transition between open and closed configurations more easily, thereby avoiding abnormal leaflet motion in low-pressure gradient implant locations. Moreover, the relatively-shallow neo-sinus region produced by the half-elliptical or semi-elliptical shape of the second portion 1214 can allow for more thorough wash-out of the neo-sinus, thereby minimizing the risk of thrombus forming therein. In some implementations, the above-described shape of the leaflets 1200 and attachment thereof to the frame 1302 can enable the valvular structure to provide a larger diameter outlet in the open configuration than what was offered with conventional valvular structures using the same annular frame. In the open configuration, the leaflets of the valvular structure can thus be positioned closer to the radially-inner circumferential surface of the frame, such that the centerline of the first portion is substantially parallel to the axial direction of the frame.

### Exemplary Prosthetic Heart Valves with Low Opening Pressure Valvular Structures

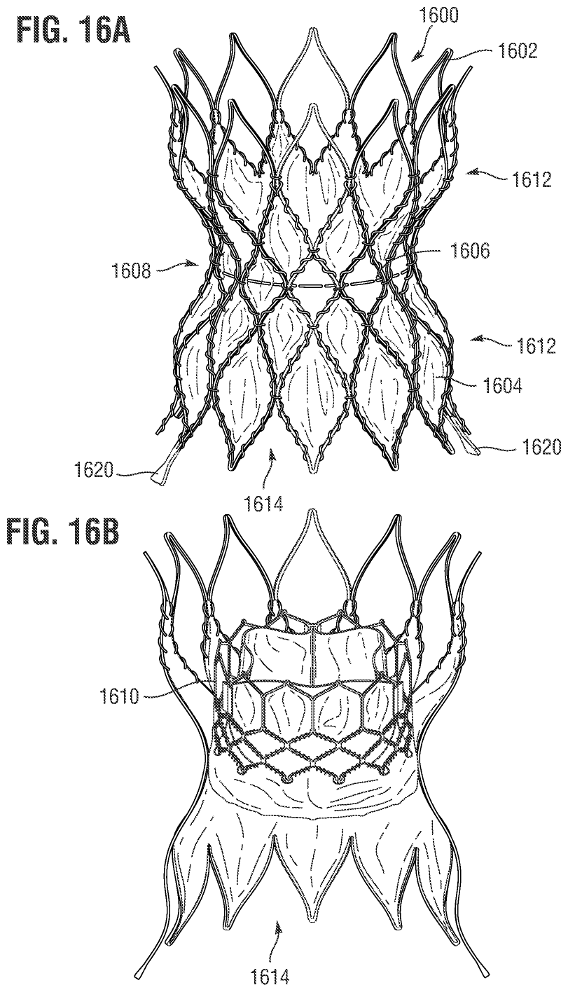

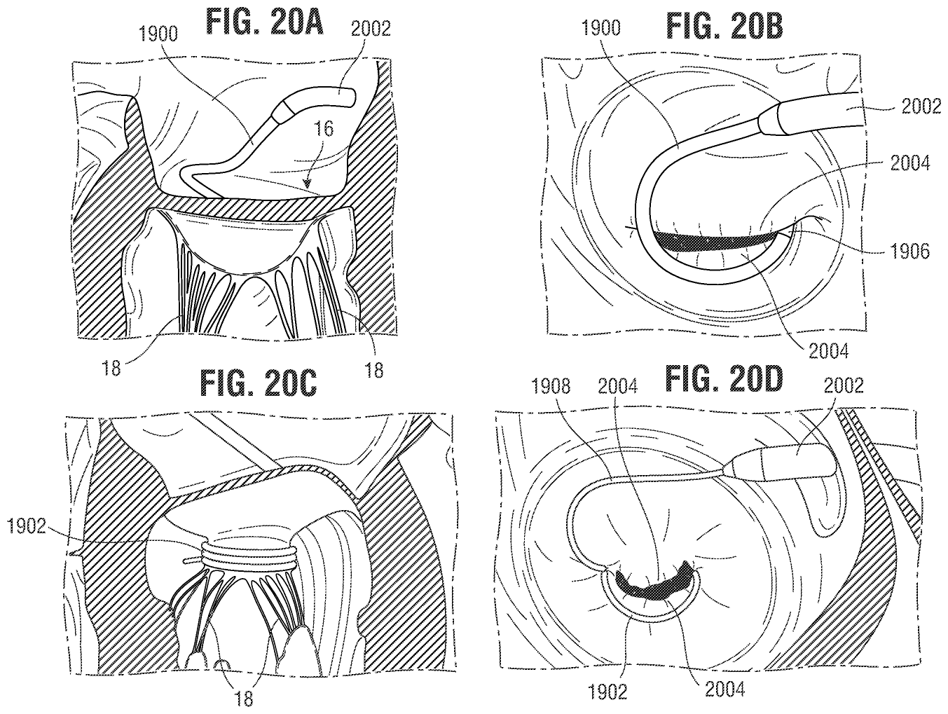

FIGS. 14A-14E illustrate various features of an exemplary prosthetic heart valve 1400 that has a low-opening-pressure valvular structure. The prosthetic heart valve 1400 can be crimped on or retained by an implant delivery apparatus in the radially-compressed configuration while the prosthetic heart valve is routed through the anatomy of a patient to the patient's heart, and then expanded to the radially-expanded configuration once the prosthetic heart valve reaches a desired implantation site within the heart. In particular examples, the prosthetic heart valve 1400 can be implanted within the native mitral valve (e.g., mitral valve 16 in FIG. 1), although it also can be implanted at other locations in the heart, including within the native pulmonary valve (e.g., pulmonary valve 30 in FIG. 1), the native tricuspid valve (e.g., tricuspid valve 26 in FIG. 1), or within a docking station (e.g., docking station 1600 in FIGS. 16A-16B) in a blood vessel leading to or from one of the heart chambers. For implantation within the native mitral valve or tricuspid valve, In some implementations, the prosthetic heart valve 1400 can be implanted within a docking station (e.g., docking station 1900 in FIGS. 19-21B) that is implanted within the native valve. Prosthetic heart valves 1400 can be implanted using any known delivery apparatus, for example, the delivery apparatus illustrated in FIG. 18.

The prosthetic heart valve 1400 can include an annular stent or frame 402, which has a first axial end 1416 and a second axial end 1418. In the depicted example, the first axial end 1416 can be an outflow end, and the second axial end 1418 can be an inflow end. The outflow end 1416 is the proximal-most end of the prosthetic valve when mounted on a delivery apparatus for delivering and implanting the prosthetic heart valve 1400 within the native aortic valve using a transfemoral, retrograde delivery approach. In other implementations, the inflow end 1418 can instead be the proximal-most end of the prosthetic valve when mounted on the delivery apparatus, depending on the particular native valve being replaced and the delivery technique that is used (e.g., trans-septal, transapical, etc.).

In some implementations, the frame 402, or components thereof (e.g., angled struts 430, axial struts 438, window 414), can be made of any of various suitable plastically-expandable materials or self-expanding materials, as known in the art. Plastically-expandable materials that can be used to form the frame 402 can include, but are not limited to, stainless steel, biocompatible high-strength alloys (e.g., a cobalt-chromium or a nickel-cobalt-chromium alloys), polymers, or combinations thereof. In particular examples, frame 402 is made of a nickel-cobalt-chromium-molybdenum alloy, such as MP35N® alloy (SPS Technologies, Jenkintown, Pa.), which is equivalent to UNS R30035 alloy (covered by ASTM F562-13, Standard Specification for Wrought 35Cobalt-35Nickel-20Chromium-10Molybdenum Alloy for Surgical Implant Applications (UNS R30035), ASTM International, West Conshohocken, Pa., 2013, which is incorporated herein by reference). MP35N® alloy/UNS R30035 alloy comprises 35% nickel, 35% cobalt, 20% chromium, and 10% molybdenum, by weight. Self-expanding materials that can be used to form the frame 402 can include, but are not limited to, nickel titanium alloy (NiTi), such as nitinol.

When constructed of a plastically-expandable material, the frame 402 (and thus the prosthetic heart valve 1400) can be crimped to the radially-compressed configuration on a delivery catheter and then expanded inside a patient by an inflatable balloon or equivalent expansion mechanism. Alternatively, when constructed of a self-expanding material, the frame 402 (and thus the prosthetic heart valve 1400) can be crimped to the radially-compressed configuration and restrained in the compressed configuration by insertion into a sheath or equivalent mechanism of a delivery catheter. Once advanced to the desired implantation site, the prosthetic heart valve can be advanced from the delivery sheath, thereby allowing the prosthetic heart valve to expand to its functional size. Further details of delivery apparatuses that can be used to deliver and implant self-expandable prosthetic valves (including any of the prosthetic valves disclosed herein when the frames are constructed of a self-expandable material such as nitinol) are disclosed in U.S. Patent Application Publication Nos. 2014/0343670 and 2010/0049313, which are incorporated herein by reference.

In some implementations, struts 430 of the frame 402 are pivotable or bendable relative to each other to permit radial expansion and contraction of the frame 102. For example, the frame 402 can be formed (e.g., via laser cutting, electroforming or physical vapor deposition) from a single piece of material (e.g., a metal tube). In other implementations, the frame 402 can be constructed by forming individual components (e.g., the struts and fasteners of the frame) and then mechanically assembling and connecting the individual components together. For example, instead of the strut structure illustrated in FIGS. 14A-14B, the frame can have individual diagonally-extending struts pivotably coupled to one another at one or more pivot joints along the length of each strut, as described in U.S. Patent Application Publication Nos. 2018/0153689, 2018/0344456, and 2019/0060057, all of which are incorporated herein by reference.

As best seen in FIG. 14B, the frame 402 can be formed with a plurality of circumferentially-spaced commissure windows 414. A valvular structure can be coupled to the frame 402 at the commissure windows 414. For example, the valvular structure can have a plurality of commissure assemblies 1412, each corresponding to a respective one of the commissure windows 414 of the frame 402. In the illustrated example of FIG. 14A-14E, the valvular structure comprises three leaflets 1410 (e.g., a tricuspid structure), and the commissure windows 414 are equally spaced at 120° intervals (i.e., 0°, 120°, and 240°) along the circumference of the frame 402. However, other spacings and numbers of commissure windows 414 are also possible. For example, In some implementations, the valvular structure comprises two leaflets (e.g., a bicuspid structure), and the commissure windows are disposed on opposite sides of the frame (e.g., aligned on a same diameter of the frame).

As shown in FIGS. 14A and 14D, prosthetic heart valve 1400 can also include one or more skirts or sealing members. For example, the prosthetic heart valve 1400 can include an inner skirt 1408 mounted on an interior of the frame 402 (e.g., radially-inward of a frustoconical wall formed by the lattice structure of the struts of the frame). The inner skirt 1408 can be a circumferential inner skirt that spans an entire circumference of the interior of the frame 402. The inner skirt 1408 can function as a sealing member to prevent, or at least reduce, perivalvular leakage (e.g., when the valve is placed at the implantation site) and as an attachment surface to anchor a portion of the leaflets 1410 to the frame 402.

Although not illustrated in FIGS. 14A-14E, the prosthetic heart valve 1400 can also include an outer skirt mounted on an exterior of the frame 402 (e.g., radially-outward of the frustoconical wall formed by the lattice structure of the struts of the frame), for example, in a manner similar to that described above for any of FIGS. 5A-9B. The outer skirt can function as a sealing member by sealing against the tissue of the native valve annulus and can help to reduce paravalvular leakage past the prosthetic heart valve 1400. The inner and outer skirts can be formed from any of various suitable biocompatible materials, including any of various synthetic materials (e.g., polyethylene terephthalate (PET)) or natural tissue (e.g., pericardial tissue). The inner skirt and/or outer skirt can be coupled to the frame 402 using sutures, adhesive, welding, and/or other means for attaching the skirts to the frame. Further details regarding the inner and outer skirts, techniques for assembling the leaflets to the inner skirt, and techniques for assembling the skirts on the frame are disclosed in U.S. Pat. No. 9,393,110, U.S. Patent Application Publication No. 2019/0192296, International Publication No. WO/2020/159783, and International Patent Application No. PCT/US2020/024559, each of which is incorporated herein by reference.

The valvular structure can be configured to allow blood flow through the frame 402 in only one direction, for example, to regulate the flow of blood through the prosthetic heart valve 1400 from the inflow end 1418 to the outflow end 1416. The valvular structure can include a plurality of leaflets 1410, each made of a flexible material. The leaflets 1410 can transition between an open configuration, where blood flows through the valve 1400 via a flow channel formed by the leaflets, and a closed configuration, where the leaflets occlude blood flow through the valve 1400. The leaflets 1410 can be made from in whole or part, biological materials, bio-compatible synthetic materials, or other such materials. Suitable biological materials can include, for example, bovine pericardium (or pericardium from other sources). In some implementations, the leaflets 1410 can have a reinforcing member (e.g., fabric strip) at the cusp edge 1466 where the leaflets 1410 are attached to the inner skirt 1408.

Similar to the configuration illustrated in FIG. 13A, leaflet 1410 has a first portion 1454, a pair of first tabs 1458 on opposite ends of the leaflet 1410 with respect to a centerline 1452 of the first portion 1454, and a second portion 1494, as best shown in FIG. 14C. The first portion 1454 defines a first edge 1456 that can extend between the first tabs 1458. Each first tab 1458 can have at least an outer edge 1460 and a base edge 1462. The outer edges 1460 of the first tabs 1458 can be substantially parallel to each other, e.g., both edges 1460 extend along direction 1490. The outer edges 1460 (and direction 1490) can also be parallel to the centerline 1452 of the first portion 1454. The leaflet 1410 can also have a pair of second tabs 1494 on opposite ends of the leaflet 1410 with respect to centerline 1452. Each second tab 1494 can be separated from a corresponding first tab 1458 by a gap or cutout 1493. The second tabs 1494 can also have outer edges 1492 that are substantially parallel to each other. The outer edges 1492 of the second tabs 1494 may also be substantially parallel to outer edges 1460 and/or centerline 1452.

The second portion 1464 defines a cusp edge 1466 that extends between base edges 1462 of first tabs 1458 and that is curved along its entire length (or substantially along the entire length) between the base edges 1462. The cusp edge 1466 can have an apex 1480 that coincides with centerline 1452 of the first portion 1454. The second portion 1464 has a half-elliptical shape, with the cusp edge 1466 following a curvature of a half-ellipse along its entire length from base edge 1462 of one of the first tabs 1458 to the corresponding base edge 1462 of the other of the first tabs 1458. A major axis 1468 of the half-ellipse is arranged to substantially coincide with the base edges 1462, as illustrated in FIG. 14C. Accordingly, a tangent 1474 to the cusp edge 1466 at location 1472 where the cusp edge 1466 intersects with the base edge 1462 of the first tabs 1458 is substantially parallel to outer edges 1460 of the tabs 1458 (e.g., parallel to direction 1490) and substantially perpendicular to the base edges 1462.

The cusp edges 1466 of each leaflet 1410 can be sutured to inner skirt 1408, for example, along suture line 1420. The sutures can be in-and-out sutures (e.g., using Ethibond thread or the like) extending through each leaflet 410, the skirt 408 and an optional reinforcing strip disposed over or adjacent to cusp edge 1466. As discussed above with respect to FIGS. 13A-13C, the suture line 1420 can extend continuously from the apex 1480 of the cusp edge to a location substantially at or adjacent to the base edges 1462 of the first tabs 1458 forming the commissure assembly 1412.

As shown in FIGS. 14B and 14D, each commissure window 414 can be formed within or part of a lattice structure formed by axial struts 438 and angled struts 430. The struts 430, 438 of the frame can form circumferentially-extending rows of open cells 444, 446, 448, 450, with the row of cells 450 closest to the outflow end 416 have an open area greater than the other cells. In the illustrated example of FIG. 14B, each commissure window 414 can have a rectangular construction, with a central opening defined by a pair of side struts 432 (e.g., extending primarily along an axial direction of the frame 402) and a pair of cross-bars (e.g., extending primarily along a circumferential direction of the frame 402 at opposite ends of the struts 432). Other shapes and configurations for commissure window 414 are also possible. For example, instead of a rectangular opening, the commissure window can define an opening that is square, oval, square-oval, triangular, L-shaped, T-shaped, C-shaped, H-shaped, or any other shape.

FIG. 14D shows an exemplary approach for securing a commissure assembly 1412 of the valvular structure to commissure window 414 of the annular frame 402. The commissure assembly 1412 include parts of the first tabs 1458 that extend through window 414 and are splayed along a circumferential direction of the frame 402 to form a T-shape. The first tabs 1458 can wrapped in, or at least partially covered by, a coupling member 1486 (e.g., flexible cloth). First parts 1494a of the second tabs 1494 folded against the inner surface of the respective leaflet 1410, and second parts 1494b of the second tabs 1494 can be folded to extend along the circumferential direction of the frame 402 and face the first tab portions 1458. The second parts 1494b can be coupled to the coupling member 1486 and/or the respective first tabs 1458 via one or more sutures 1484.

The first parts 1494a of the second tabs 1494 can form a multi-layer structure of leaflet material just inside the commissure window 414, which structure may be more resistant to bending or articulating than the leaflet portions radially inward, thereby causing the leaflets 410 to articulate primarily at inner edges 1482 of the first parts 1494a of the second tabs 1494. The first parts 1494a of the second tabs 1494 can thus help the leaflets to avoid contact with or damage from the frame 402 during normal operation of the valvular structure. Detailed instructions for folding the second tabs 1494 to yield the arrangement of first parts 1494a and second parts 1494b illustrated in FIG. 14D can be found in, for example, U.S. Pat. No. 9,393,110, which is incorporated herein by reference.

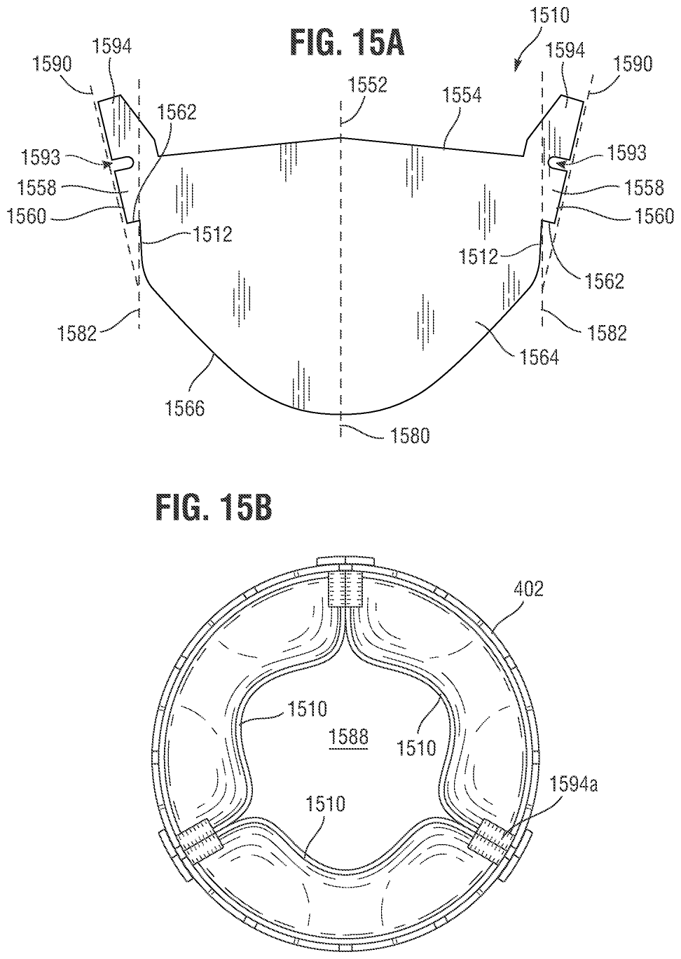

For comparison, FIG. 15A shows a design of a conventional leaflet 1510 having an upper free edge portion 1554 that extends between a pair of first tabs 1558 on opposite ends of the leaflet 1510 with respect to leaflet centerline 1552. At the lower edge of the leaflet 1510, a lower edge portion 1564 extends between the respective ends of tabs 1558. However, in contrast to the second portion 1464 of FIG. 14C, the lower edge portion 1564 in FIG. 15A is not curved along its entire length. Rather, the lower edge portion 1564 includes a substantially-straight edge portions 1512 extending from the base edges 1562 of the first tabs 1558 and a substantially V-shaped intermediate edge portion 1566 between the straight edge portions 1512. The substantially V-shaped intermediate edge portion can have a smooth curved apex portion 1580 and oblique portions connecting the apex portion 1580 to the respective straight edge portion 1512. The oblique portions can have a greater radius of curvature than the apex portion 1580.

Similar to the leaflet 1410 of FIG. 14C, the leaflet 1510 can also have a pair of second tabs 1594 separated from the first tabs 1558 by a gap or cutout 1593. However, outer edges 1560 of the first tabs 1558 extending along direction 1590 are not parallel to each other, nor to centerline 1552. Moreover, the tangent 1582 to the lower edge portion 1564 where the lower edge portion 1564 intersects with the base edge 1562 of the first tab 1558 is not parallel to the outer edge 1560 of the first tabs 1558, nor perpendicular to base edge 1562 of the first tab 1558. Finally, the suture line used to attach the lower edge portion 1564 to the inner skirt of the valve may extend from the apex 1580 to only the lower end of the straight edge portion 1512, with the straight edge portion 1512 being separately coupled to the straight edge portion 1512 of an adjacent leaflet, for example, using a comb stitch. As a result of the shape of leaflet 1510, construction and attachment of the commissure assembly to the frame 402, and the suture line coupling the lower edge portion 1564 to the inner skirt, the valvular structure may have a more difficult time transitioning between open and closed configurations in a low-pressure gradient position. The valvular structure formed by conventional leaflets 1510 may also result in an outlet open area 1588 in the open configuration that is significantly less than an inlet area of the frame.

In contrast, the example of FIGS. 14A-14E provides a valvular structure that transitions between open and closed configurations more easily, thereby avoiding abnormal leaflet motion in low-pressure gradient implant locations. For example, the combination of the half-elliptical shape of the second portion 1464, the parallel arrangement of outer edges 1460 of first tabs 1458 and the tangents 1474 of the cusp edge 1466, and the continuous suture line 1420 extending to (or as close as possible to) the base edges 1462 of the first tabs 1458 can allow can enable such easier transitions, thereby reducing the likelihood of abnormal leaflet motion due to the lower pressure gradient. As compared to the conventional leaflet 1510, the shallower and more rounded neo-sinus region formed by the half-elliptical shape of edge 1466 of the leaflet 1410 attached to the inner skirt can also aid in effective wash-out of the neo-sinus region. The neo-sinus wash-out together with the avoidance of leaflet stasis in low-pressure implantation environments can further reduce the risk of thrombosis. In some implementations, the easier transition to the open configuration may also result in a larger outlet open area 1488, as shown in FIG. 14E. As such, the size of inwardly projecting first part 1494a in FIG. 14D can be reduced as compared to the inwardly projecting first part 1594a in FIG. 15B or eliminated altogether, such that the valvular structure formed by leaflets 1410 can take full advantage of the larger outlet open area 1488. In some implementations, the thickness of the leaflets 1410 can be reduced as compared to conventional leaflets, for example, to allow the leaflets to be more compliant and thus transition between open and closed configurations more readily. For example, the conventional leaflet 1510 can have a thickness of 0.016-0.020 inches (406 μm to 508 μm), while the leaflets 1410 can have a thickness of about 0.012 inches (305 μm).

Although the above discussion of FIGS. 14A-14E specifies a particular configuration for prosthetic heart valve 1400, other examples of a prosthetic heart valve, disclosed herein or otherwise, can include any of the innovations and variations discussed above with respect to FIGS. 3A-13C or later discussed below with respect to FIGS. 16A-21B. For example, the modified valvular structure formed by leaflets 1410 can be adapted to replace valvular structure 918 in the prosthetic heart valve 900 of FIGS. 10A-10C, thereby avoiding abnormal leaflet motion when the prosthetic heart valve 900 is mounted in low-pressure gradient implant locations.

FIGS. 16A-16B illustrate various features of a docking station (also referred to herein as a valve dock or docking device) that can be used with a prosthetic heart valve, such as valve 1400, in a low-pressure implantation location. Docking station 1600 can be made from a resilient or compliant material and can be designed to accommodate large variations in the anatomy. For example, the docking station 1600 can be made from a highly flexible metal (e.g., nitinol), metal alloy, polymer, or an open cell foam. The docking station 1600 may be self-expanding (e.g., by being formed of a shape memory alloy), manually expandable (e.g., expandable via balloon), or mechanically expandable.

As illustrated in FIG. 16A, the docking station 1600 can include a frame 1602 forming one or more cells. A band 1606 can extend about a waist or narrow portion 1608, or can be integral to the waist to form a non-expandable or substantially non-expandable valve seat. The band 1606 can stiffen the waist and, once the docking station is deployed and expanded, makes the waist/valve seat relatively non-expandable in its deployed configuration. As shown in FIG. 16B, a valve 1610 (e.g., prosthetic heart valve 1400, or any other valve disclosed herein or known in art) can be secured to the docking station 1600 by expansion of the frame of the prosthetic valve 1610 into narrow portion 1608, which forms a valve seat. The band 1606 can be made of PET, one or more sutures, fabric, metal, polymer, a biocompatible tape, or any other relatively non-expandable material known in the art capable of restricting a shape of the valve seat and hold the installed valve 1610 therein.

The docking station 1600 can be implanted in a blood vessel within the patient's vascular system, for example, a blood vessel leading to or from a chamber of the heart (e.g., the pulmonary artery, the inferior vena cava, or the superior vena cava). The frame 1602 can include one or more retaining portions 1620, which can have an outwardly curving flare designed to help secure the docking station 1600 within a blood vessel. The docking station 1600 can thus provide a mounting location for the prosthetic heart valve within the blood vessel rather than within one of the native heart valves. The docking station can have an impermeable material 1604 coupled to the frame 1602 to form sealing portions. For example, the sealing portion can comprise radially-outward-extending portions 1612, and the impermeable material 1604 can extend from at least portions 1612 to the valve seat, thereby making the docking station 1600 impermeable to blood flow and directing blood flowing into the inflow end 1614 of the docking station to the valve 1610 installed in the valve seat.

While the impermeable material 1604 may be impermeable to blood flow, it may still allow tissue ingrowth from the surrounding native tissue of the blood vessel. In some implementations, the impermeable material 1604 can be replaced with a hermetic layer, for example, as described above with respect to FIGS. 3A-11B. Alternatively, or additionally, the inner skirt, the outer skirt, or both of the prosthetic heart valve 1610 can compromise one or more hermetic layer to act as a barrier to further tissue ingrowth from the docking station to the leaflets of the prosthetic valve 1610. Moreover, since any valve mounted in the docking station 1600 may experience a relatively low pressure gradient, prosthetic heart valve 1610 preferably comprises a valvular structure designed to more readily transition between the open and closed configurations, for example, as described above with respect to FIGS. 13A-14E.

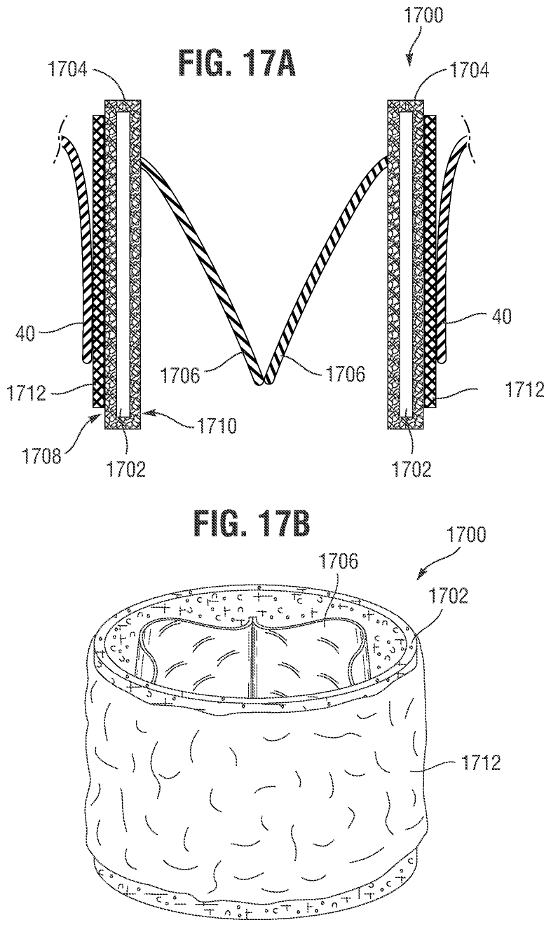

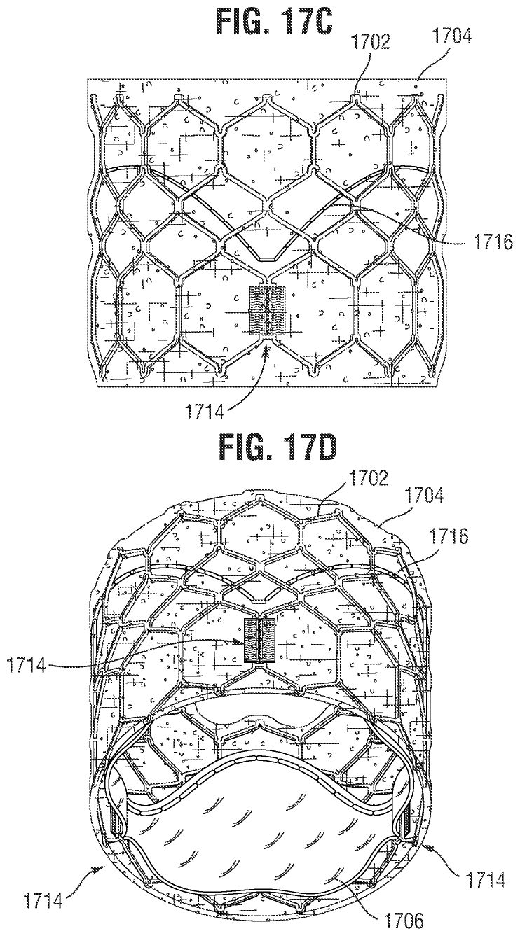

Further details regarding the construction and use of docking systems, which may be adapted to docking station 1600 and/or used with prosthetic valve 1610 or any other exemplary valve, are disclosed in U.S. Pat. No. 10,363,130 and U.S. Patent Application Publication No. 2019/0000615, each of which is incorporated herein by reference. Although the above discussion of FIGS. 16A-16B specifies a particular configuration for the docking station 1600 and prosthetic heart valve 1610, other examples of a docking station and/or a prosthetic heart valve, disclosed herein or otherwise, can include any of the innovations and variations discussed above with respect to FIGS. 3A-14E or later discussed below with respect to FIGS. 17A-21B.

Moreover, while specific examples of docking stations and prosthetic heart valves are discussed above, the provision of low pressure opening valvular structures is applicable to a wide variety of prosthetic valves and/or associated docking stations. For example, the existing valvular structures of the prosthetic heart valves disclosed in any of U.S. Pat. Nos. 6,730,118, 7,101,396, 7,393,360, 7,510,575, 7,993,394, 8,652,202, 8,992,608, 9,339,382, and 10,603,165, U.S. Patent Application Publication Nos. 2018/0325665, 2018/0344456, and 2019/0060057, and International Publication No. WO/2020/081893, all of which are incorporated by reference herein, can be replaced with the disclosed valvular structure. Alternatively, or additionally, any of the prosthetic heart valves disclosed in any of U.S. Pat. Nos. 6,730,118, 7,101,396, 7,393,360, 7,510,575, 7,993,394, 8,652,202, 8,992,608, 9,339,382, and 10,603,165, U.S. Patent Application Publication Nos. 2018/0325665, 2018/0344456, and 2019/0060057, and International Publication No. WO/2020/081893, can be modified for operating in low-pressure gradient implant locations in accordance with the teachings of the present disclosure.

### Exemplary Prosthetic Valve with Hermetic Layer and Low-Opening Pressure Valvular Structure

FIGS. 17A-17D illustrate various features of an exemplary prosthetic heart valve 1700 having one or more hermetic layers and a low-opening pressure valvular structure, for example, for implantation at the mitral location. Similar to the prosthetic heart valve 800 illustrated in FIGS. 9A-9B, the exemplary prosthetic heart valve 1700 of FIGS. 17A-17D has an annular frame 1702, a valvular structure comprising a plurality of leaflets 1706, and an encapsulating layer 1704 comprising one or more hermetic layers. The encapsulating layer 1704 can fill open cells of the frame 1702 and enclose struts of the frame 1702, such that the encapsulating layer 1704 surrounds the entire annular frame 1702 on all sides, thereby enclosing or embedding the annular frame 1702 within layer 1704. The encapsulating layer 1704 forms a radially-inner surface that acts as a hermetic inner skirt 1710 and a radially-outer surface that acts as a hermetic outer skirt 1708. The encapsulating layer 1704 thus provides both barriers both internal and external to the frame 1702 against tissue ingrowth, thus reducing the likelihood of pannus formation.

The encapsulating layer 1704 can be formed by pre-forming sublayers, disposing the sublayers on opposite sides of the frame 1702, and then coupling the sublayers together to embed the annular frame 1702 therein. For example, a first extruded sublayer can be disposed on a radially-inner circumferential surface of the annular frame 1702 and a second extruded sublayer can be disposed on a radially-outer circumferential surface of the annular frame 1702. The first and second sublayers can then be joined with the struts of the frame 1702 therebetween, for example, by fusing, melting, welding, or the like. Alternatively, the encapsulating layer 1704, or a portion thereof, can be formed directly on the frame 1702, for example, by dip coating, spray coating, electrospinning, or the like.

In some implementations, the encapsulating layer 1704 comprises a hermetic layer as described in detail in the sections above. For example, the encapsulating layer 1704 is formed of a hydrophobic polymer material and can be substantially non-porous, or can have pores therein of sufficiently small size that discourage cellular ingrowth (e.g., 20 μm or less in size, 10 μm or less in size, 8 μm or less in size, or even 5 μm or less in size). Exemplary materials for the hermetic layer include PTFE, ePTFE, urethane, PU, TPU, silicone, or combinations or copolymers thereof. For example, in an exemplary implementation, frame 1702 is encapsulated by urethane layers electrospun with ePTFE. In another exemplary implementation, frame 1702 is encapsulated by a copolymer of silicone and TPU, which copolymer may be coated on the frame 1702. Further details regarding materials for encapsulating layers, and techniques for encapsulation and for attaching leaflets to the encapsulating layers, which may be employed in valve 1700 or any other exemplary valve, can be found in U.S. Pat. No. 8,945,209 and U.S. Patent Application Publication No. 2020/0155306, both of which are incorporated by reference in their entireties.

In the illustrated example of FIGS. 17A-17B, a separate outer skirt 1712 is provided over encapsulating layer 1704. Outer skirt 1712 can be disposed on a radially-outer surface of the encapsulating layer 1704 and coupled thereto by sutures or any other coupling means. For example, the outer skirt 1712 can be made from any of a variety of woven, knitted, or crocheted fabrics, with the radially outer surface being a plush nap or pile of the fabric. Exemplary fabrics having a pile include velour, velvet, velveteen, corduroy, terrycloth, fleece, etc. Alternatively, or additionally, the outer skirt 1712 can comprise a non-woven fabric (e.g., felt) or fibers (e.g., non-woven cotton fibers). Alternatively, or additionally, the outer skirt 1712 can be formed as or constructed from porous or spongey materials such as, for example, any of a variety of compliant polymeric foam materials, or woven fabrics, such as woven PET. The materials selected for the outer skirt 1712 can contribute to improved compressibility and shape memory properties of the outer skirt. For example, a pile layer can be compliant such that is compresses under load (e.g., when in contact with native tissue, other implants, or the like) but otherwise returns to its original size and/or shape when the load is removed. Accordingly, the materials selected for the outer skirt 1712 may permit and even encourage tissue ingrowth. However, the encapsulating layer 1704 acts as a barrier to otherwise prevent any tissue ingrowth from reaching the leaflets 1706 of the valvular structure.

The leaflets 1706 of the valvular structure are coupled to the frame 1702 via commissure assemblies 1714 coupled to respective commissure windows of the frame. In the illustrated example of FIG. 17B-17D, the valvular structure comprises three leaflets 1706 (e.g., a tricuspid structure), and the commissure windows are equally spaced at 120° intervals (i.e., 0°, 120°, and 240°) along the circumference of the frame 1702. However, other spacings and numbers of commissure windows are also possible. The valvular structure can be configured to allow blood flow through the frame 1702 in only one direction, for example, to regulate the flow of blood through the prosthetic heart valve 1700 from the inflow end to the outflow end. The leaflets 1706 transition between an open configuration, where blood flows through the valve 1700 via a flow channel formed by the leaflets, and a closed configuration, where the leaflets 1706 occlude blood flow through the valve 1700. The leaflets 1706 can be made from in whole or part, biological materials, bio-compatible synthetic materials, or other such materials. Suitable biological materials can include, for example, bovine pericardium (or pericardium from other sources). The leaflets 1706 can have a shape and arrangement similar to leaflets 1410 described above with respect to FIGS. 14C-14E, for example, with outer edges of the tabs being substantially parallel, with the cusp edge having a half-elliptical or semi-elliptical shape, and with the cusp edge at the base edge of the tabs having a tangent substantially parallel to the outer edges of the tabs.

To allow the commissure assemblies of the valvular structure to pass through and mount to commissure windows of the frame 1702, openings can be created in the encapsulating layer 1704 at locations corresponding to the windows. In some implementations, the openings can be created after the encapsulation layer 1704 is formed on the annular frame 1702, for example, by cutting away the layer 1704 in a region surrounding window and/or by piercing through the layer 1704 covering the opening of the window. Alternatively, In some implementations, the openings can be created during formation of the encapsulation layer 1704 on the annular frame 1702, for example, by covering the windows during the encapsulation process, inserting a temporary sacrificial member within the window openings during the encapsulation process, or otherwise preventing material from forming over and obstructing windows during the encapsulation process. Alternatively, In some implementations, the openings can be created in one or more of the sublayers used to form the encapsulation layer 1704 prior to encapsulation of the annular frame 1702. The commissure assemblies can otherwise be attached to windows of frame 1702 in a manner similar to that described above for FIGS. 5E and 6C.

The leaflets can be sutured to the encapsulating layer 1704, for example, at radially inner surface 1710, in a manner similar to that described above for FIGS. 5A-5B. However, in contrast to the valve of FIGS. 5A-5B, the prosthetic valve 1700 has a suture line 1716, which attaches the cusp edge of the leaflets 1706 to the encapsulating layer 1704, extending continuously from an apex of the cusp edge to a location substantially at or adjacent to the commissure assembly attached to the commissure window (e.g., at or as close as possible to base edges of the leaflet tabs inserted into the window). Similar to the configuration of FIGS. 14A-14E, the valvular structure of prosthetic valve 1700 transitions between open and closed configurations more easily, thereby avoiding abnormal leaflet motion in low-pressure gradient implant locations. Moreover, the leaflets 1706 can form shallower and more rounded neo-sinus region, which may encourage wash-out of the neo-sinus region. The neo-sinus wash-out together with the avoidance of leaflet stasis in low-pressure implantation environments can further reduce the risk of thrombosis.

Although the above discussion of FIGS. 17A-17D specifies a particular configuration for prosthetic mitral valve 1700, other examples of a prosthetic heart valve, disclosed herein or otherwise, can include any of the innovations and variations discussed above with respect to FIGS. 3A-17D or later discussed below with respect to FIGS. 18-21B.

FIG. 18 shows an exemplary delivery apparatus 1800 that can be used to deliver and implant prosthetic heart valve 1700, or any other exemplary prosthetic heart valve. Delivery apparatus 1800 includes a handle 1802 that can be disposed external to a patient and used to articulate a distal end portion 1806 of an elongated shaft 1812 within the patient. The prosthetic heart valve 1700 can be disposed on the distal end portion 1806 in a radially-compressed state. For example, the prosthetic valve 1700 can be crimped on an inflatable balloon 1804 or another type of expansion member that can be used to radially expand the prosthetic valve 1700. The distal end portion 1806, including prosthetic valve 1700, can be advanced through the vasculature to a selected implantation site (e.g., within a native mitral valve and/or within a previously implanted host valve). Although not specifically illustrated in FIG. 18, it should be appreciated that the delivery apparatus 1800 can be advanced over a guidewire, and that the delivery apparatus 1800 can include an innermost shaft that defines a lumen for the guidewire, as is known in the art. The prosthetic valve 1700 can then be deployed at the implantation site, such as by inflating the balloon 1804. Further details of delivery apparatuses that can be used to deliver and implant plastically-expandable prosthetic heart valves, such as the prosthetic valve 1700 (or any other of the prosthetic heart valves disclosed herein), are disclosed in U.S. Patent Application Publication Nos. 2017/0065415, 2016/0158497, and 2013/0030519, which are incorporated herein by reference.

If the prosthetic valve 1700 being implanted is a self-expandable prosthetic valve, the prosthetic valve can be retained in a radially compressed configuration within a delivery capsule or sheath of the delivery apparatus 1800 when inserted into and advanced through the patient's vasculature to the desired implantation site. Once positioned at the desired implantation site, the prosthetic valve can be deployed from the delivery capsule, which allows the prosthetic valve to self-expand to its radially-expanded, functional size within the native valve or a previously implanted host valve. Further details of delivery apparatuses that can be used to deliver and implant self-expandable prosthetic valves (including any of the prosthetic valves disclosed herein when the frames are constructed of a self-expandable material such as Nitinol) are disclosed in U.S. Patent Application Publication Nos. 2014/0343670 and 2010/0049313, which are incorporated by reference herein.

When prosthetic valve 1700 is implanted at the mitral location, an anchoring or docking device (e.g., a docking station or valve dock) can be used. For example, FIG. 19 shows an exemplary docking device 1900 having a coil or coiled portion with a plurality of turns extending along a central axis of the docking device. The coil or coiled portion can be continuous and can extend generally helically, with various differently sized and shaped sections. For example, the docking devices 1900 can be configured to fit at the mitral position, but can also be shaped and/or adapted similarly or differently in other implementations for better accommodation at other native valve positions, such as at the tricuspid valve. Advantageously, the geometry of docking device 1900 can provide for engagement with the native anatomy that increases stability and reduces relative motion between the docking device 1900, the prosthetic valve 1700 docked therein, and the native anatomy. Reduction of such relative motion may prevent material degradation of components of the docking device 1900 and/or the prosthetic valve 1700 docked therein and can prevent damage/trauma to the native tissues.

The docking device 1900 can include a central region 1902 with a coil, coiled portion, or multiple coils. The coiled portion or coils of the central region 1902 can be similarly sized and shaped or vary in size and/or shape. For example, the central region 1902 can have three or approximately three full coil turns having substantially equal inner diameters. The central region 1902 of the docking device 1900 serves as the main landing region or holding region for holding the expandable prosthetic valve when the docking device 1900 and the prosthetic valve are implanted into a patient's body. The coiled portion or coil(s) of the central region 1902 can also be referred to as the “functional coils” or “functional turns,” since the properties of these coils contribute the most to the amount of retention force generated between the valve prosthesis, the docking device 1900, and the native mitral leaflets and/or other anatomical structures.

In the illustrated example of FIG. 19, the docking device 1900 can also have a distal or lower region 1904. The distal region 1904 has a leading coil/turn (sometimes referred to as an encircling turn or a leading ventricular coil/turn), which can have a diameter that is greater than the diameter of the functional coils/turns or of the coils/turns of central region 1902. The diameter or width of the encircling turn or leading coil/turn (e.g., ventricular coil/turn) of the lower region 1904 can be selected to be larger so as to more easily navigate a distal or leading tip 1906 of the docking device 1900 around and encircle the features of the native anatomy (e.g., leaflets and/or chordae tendineae).

Once the distal tip 1906 is navigated around the desired native anatomy, the remaining coils of the docking device 1900 can also be guided around the same features. In some implementations, the size of the other coils can be reduced sufficiently to cause the corralled anatomical features to be pulled radially inwardly or slightly radially inwardly. In the illustrated example of FIG. 19, the docking device 1900 further includes an enlarged proximal or upper region 1908 having a stabilizing coil/turn (e.g., which can be an atrial coil/turn) of the docking device 1900. During a transient or intermediate stage of the implantation procedure, for example, during the time between the deployment and release of the docking device 1900 and final delivery of the prosthetic valve, there is a possibility that the coil could be shifted and/or dislodged from its desired position or orientation, for example, by regular heart function. The stabilization feature or coil can be used to help stabilize the docking device in the desired position during the intermediate stage. For example, the docking device 1900 includes the upper region 1908 with an enlarged stabilization coil/turn intended to be positioned in the circulatory system (e.g., in the left atrium) such that it can stabilize the docking device 1900. For example, the upper region 1908 or stabilization coil/turn can be configured to abut or push against the walls of the circulatory system (e.g., against the walls of the left atrium), in order to maintain a desired position of the docking device 1900 prior to the implantation of the prosthetic valve.

The stabilization coil/turn (e.g., atrial coil/turn) at the upper region 1908 of the docking device 1900 can extend up to about one full turn or rotation, and terminates at a proximal tip 1910. The radial size of the stabilization coil/turn (e.g., atrial coil) at the upper region 1908 can also be significantly larger than the size of the functional coils in the central region 1902, so that the stabilization coil/turn flares or extends sufficiently outwardly in order to contact the walls of the circulatory system (e.g., the walls of the left atrium). The proximal tip 1910 of the upper region 1908 can include an eyelet or eyehole, for example, to secure the docking device 1900 to a delivery system.

In implementations where the docking device 1900 is used at the mitral position, the docking device can first be advanced and delivered to the native mitral valve annulus, and then set at a desired position, prior to implantation of the prosthetic heart valve. In some implementations, the docking device 1900 is flexible and/or made of a shape memory material, so that the coils of the docking device 1900 can be straightened for delivery via a transcatheter approach as well. In some implementations, the coil is made of another biocompatible material, such as stainless steel. Some of the same catheters and other delivery tools can be used for both delivery of the docking device 1900 and the prosthetic valve 1700, without having to perform separate preparatory steps, simplifying the implantation procedure for the end user. Further details of docking stations and implantation thereof, which may be employed with prosthetic valve 1700 or any other exemplary valve, are disclosed in U.S. Pat. No. 10,463,479 and International Application No. PCT/US2020/036577, both of which are incorporated by reference herein.

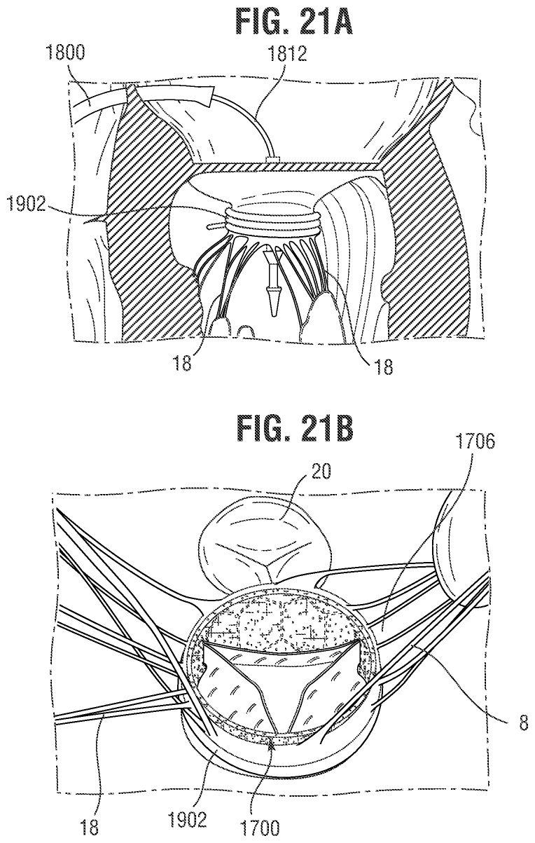

FIGS. 20A-21B illustrate various stages in implanting a docking device and prosthetic heart valve in a native mitral valve. Referring initially to FIGS. 20A-20B, an initial stage of delivering the docking device 1900 to the mitral position and implanting the docking device 1900 in the native mitral valve 16 is shown. A distal end portion of a delivery system 2002 is advanced to the native mitral valve 16 of a heart of a patient. The docking device can be positioned at the native mitral valve 16, such that the distal tip 1906 extend through the valve 16 into the left ventricle. A sleeve shaft of the delivery system 2002 may be retracted in a proximal direction to expose the docking device 1900 and/or the docking device can be pushed from the delivery system, thereby allowing the shape memory material of the docking device 1900 to adopt the coiled shape of the central region 1902, which can encircle the chordae tendineae 18 within the left ventricle, as illustrate in FIGS. 20C-20D. Once fully deployed within the mitral valve 16, the docking device 1900 can be decoupled from the delivery system 2002. The same or another delivery system (e.g., delivery apparatus 1800) can then be used to deliver prosthetic heart valve 1700 between the native leaflets 2004 of the mitral valve 16 and to expand the prosthetic heart valve 1700 to mount it within the docking device 1900, as shown in FIGS. 21A-21B.

### Exemplary Outer Skirt for Prosthetic Heart Valves

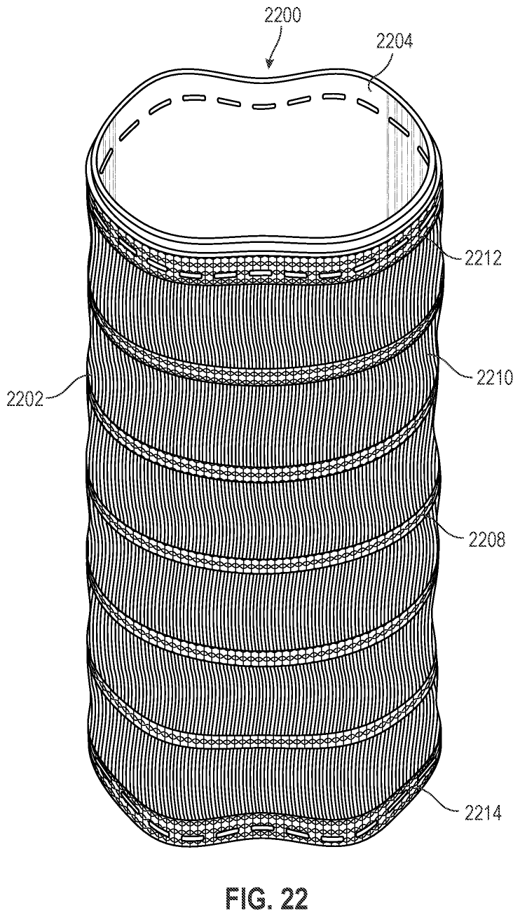

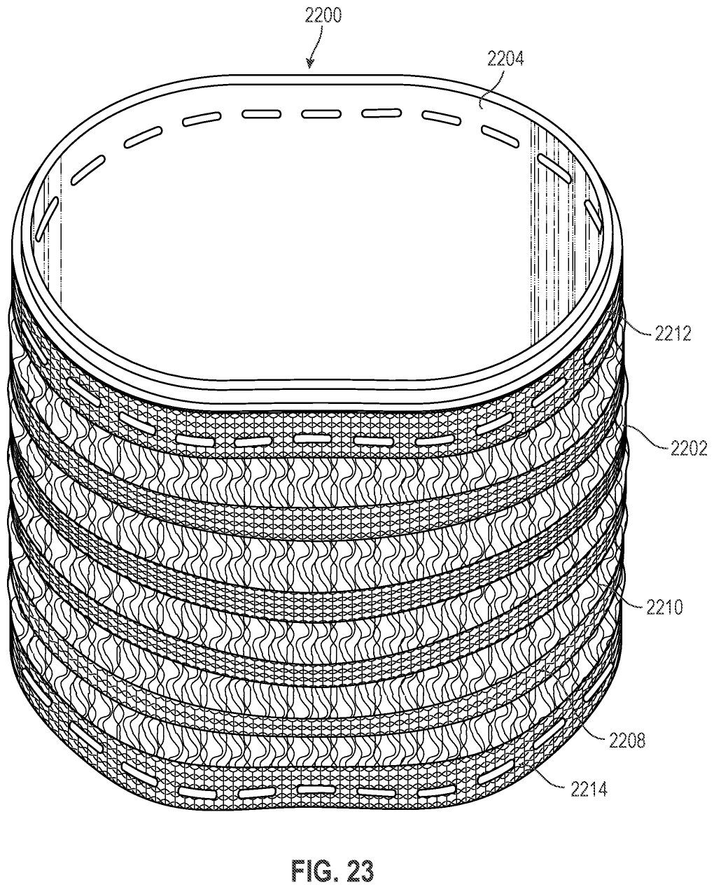

FIGS. 22 and 23 illustrate an outer skirt 2200 that can be mounted on an outside surface of a prosthetic heart valve, according to another implementation. The outer skirt 2200 provides a cushion that can contact surrounding tissue after implanting the prosthetic heart valve within a native anatomy. The cushion can reduce damage to the surrounding tissue due to movement or friction between the tissue and the surfaces of the prosthetic heart valve. The outer skirt 2200 can also reduce paravalvular leakage. The skirt 2200 has particular applicability to a prosthetic heart valve that is implanted within a docking device 1900 in the native mitral valve, such as the prosthetic heart valve 1700 shown in FIG. 21B, although prosthetic valves having a skirt 2200 can be implanted at other locations, with or without a docking device.

In the implementation illustrated in FIGS. 22 and 23, the outer skirt 2200 includes an outer fabric layer 2202 and an inner fabric layer 2204. Each of the outer fabric layer 2202 and inner fabric layer 2204 can have a tubular shape. The inner fabric layer 2204 is disposed on an inner surface of the outer fabric layer 2202. In some examples, the inner fabric layer 2204 can be attached to the outer fabric layer 2202 (e.g., by sutures, ultrasonic welding, adhesive, or with lamination techniques). In use, the outer skirt 2200 is disposed around the frame of the prosthetic heart valve such that the inner fabric layer 2204 is interposed between the frame and the outer fabric layer 2202. The outer fabric layer 2202 includes the cushion that can protect surrounding tissue from damage after valve implantation.

In one example, the outer skirt 2200 is stretchable between an elongated state corresponding to a radially compressed configuration of the prosthetic heart valve and a radially expanded state corresponding to a radially expanded configuration of the prosthetic heart valve. In the elongated state, the outer skirt 2200 can be relatively long and have a relatively narrow diameter (such as illustrated in FIG. 22). In the expanded state, the outer skirt 2200 can be relatively short and have a relatively wide diameter (such as illustrated in FIG. 23).

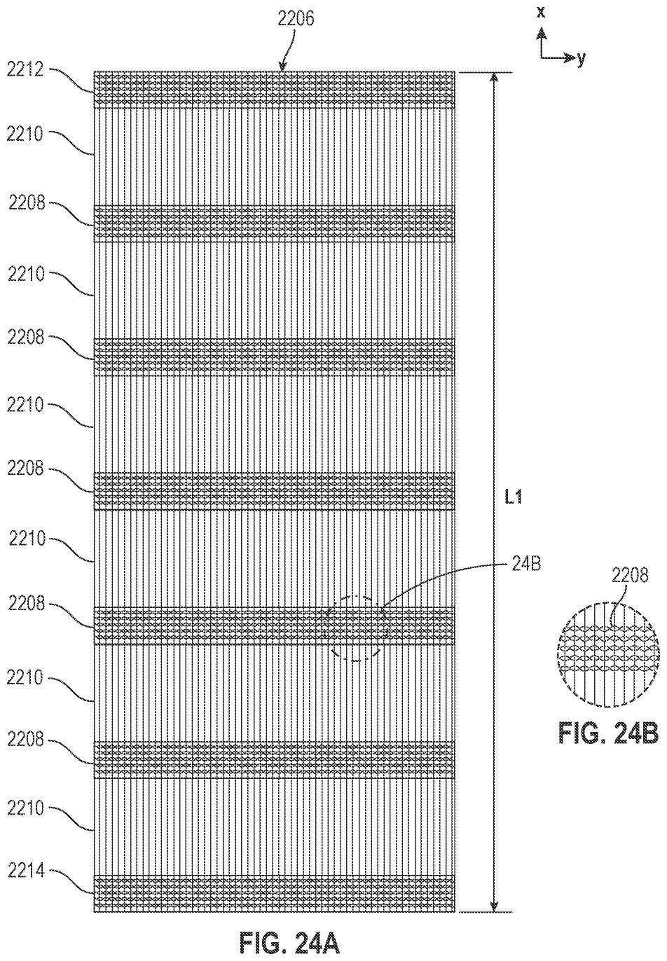

In one example, the outer fabric layer 2202 can be made with a woven fabric comprised of sections with different structures. FIGS. 24A and 24B illustrate an exemplary woven fabric 2206 having different sections that can include one or more first fabric sections 2208, one or more second fabric sections 2210, and fabric end sections 2212, 2214. The first fabric sections 2208 can have a woven structure, the second fabric sections 2210 can have a floating structure, and the fabric end sections 2212, 2214 can have a woven structure. The woven structures of the first fabric sections 2208 and the fabric end sections 2212, 2214 can be the same or can be different. The different sections 2208, 2210, 2212, and 2214 can be formed as circumferentially extending rows or stripes. The stripes of the first fabric sections 2208 can alternate with the stripes of the second fabric sections 2210 along the x-axis.

The woven fabric 2206 can be constructed using warp and weft yarns, as known in the art of weaving. In some examples, the fabric 2206 can be woven such that warp yarns run lengthwise along the fabric, while weft yarns are interlaced with the warp yarns in a crosswise direction to the warp. In the notation used in FIG. 24A, the y-axis can represent the longitudinal direction (or longitudinal grain) of the fabric, and the x-axis can represent the crosswise direction (or crosswise grain) of the fabric. An individual warp yarn can be referred to as “warp end”, and a single weft yarn extending in a crosswise direction to the warp can be referred to as a “pick” or “fill”.

In the illustrated example of FIG. 24A, the first fabric sections 2208 and the fabric end sections 2212, 2214 have a woven structure, which means that weft yarns are interlaced with warp ends in a defined pattern in these sections. The second fabric sections 2210 have a floating structure, which means that weft yarns are not interlaced with warp ends in these sections. The floating structure can be composed of floating yarns. In the example illustrated in FIG. 24A, the floating yarns are floating weft yarns (i.e., yarns extending in the crosswise direction of the fabric or along the x-axis), which can allow stretching of the fabric in the crosswise direction or along the x-axis. In an alternative example, the fabric can be woven such that the floating yarns are floating warp yarns (i.e., yarns extending in the longitudinal direction of the fabric or along the y-axis), and the stripes of the first fabric sections 2208 can alternate with the stripes of the second fabric sections 2210 along the y-axis.

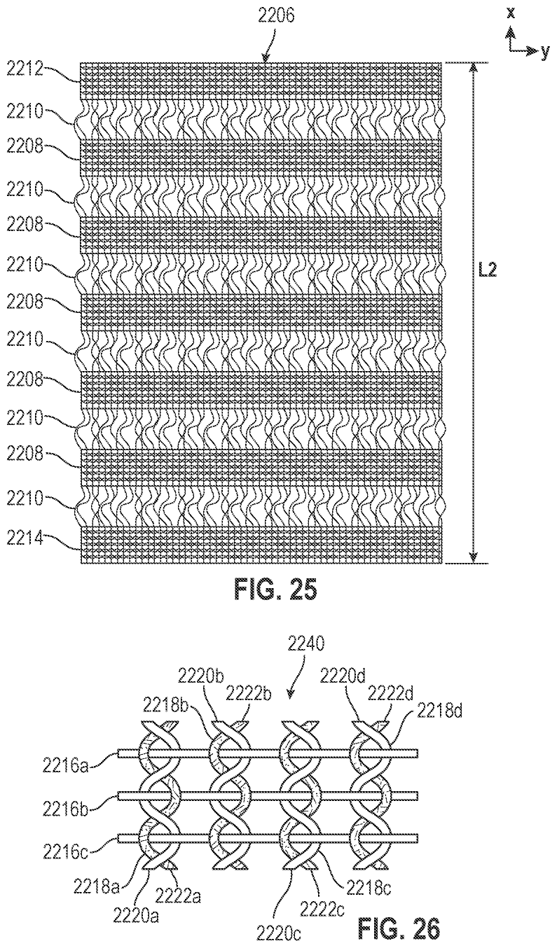

In one example, the woven fabric 2206 can have an axially elongated state with a length L1 (as shown in FIG. 24A) corresponding to a radially compressed state of the prosthetic valve and an axially foreshortened state with a length L2 (as shown in FIG. 25) corresponding to a radially expanded state of the prosthetic valve, where L1>L2. The second fabric sections 2210 with the floating structure allow adjustment of the length of the woven fabric 2206 between L1 and L2. In the elongated state of the fabric, the floating yarns making up the floating structure of the second fabric sections 2210 can be substantially parallel to the y-axis. In the foreshortened state, the floating yarns in the second fabric sections 2210 can be twisted and kinked in many directions, forming a compressible mass that can provide a cushion.

In one example, the woven structure of the first fabric sections 2208 (as well as the fabric end sections 2212, 2214) can be a leno weave structure. In leno weave, at least some of the warp ends do not lie parallel to other warp ends. Instead, some of the warp ends are twisted partly around other warp ends, forming interstices for passage of the weft yarns. The leno weave structure uses two types of warp yarns known as doup yarn and ground yarn. The doup yarn and ground yarn cross each other alternately to produce the twisted structure of the leno weave. The leno weave forms an open mesh structure that is firm, allowing the first fabric sections 2208 to serve as supports for the second fabric sections 2210 with the floating structure. The leno weave can also allow the first sections 2210 to stretch in the crosswise direction (which would be the circumferential direction when the fabric is used as the outer fabric layer of the outer skirt).

FIG. 26 illustrates three picks 2216a, 2216b, 2216c of a pure leno weave. Four paired warp ends 2218a, 2218b, 2218c, 2218d are shown for illustrative purposes. Paired warp ends 2218a includes a doup yarn 2220a and a ground yarn 2222a; paired warp ends 2218b includes a doup yarn 2220b and a ground yarn 2222b; paired warp ends 2218c includes a doup yarn 2220c and a warp yarn 2222c; and paired warp ends 2218d include a doup yarn 2220d and a ground yarn 2222d. The doup and ground yarns in each paired warp ends cross each other between the picks. The basic building block of the leno weave is completed in two picks. A woven section of a desired length can be constructed by repeating these two picks.

The woven structure of the first fabric sections 2208 is not limited to the pure leno weave structure illustrated in FIG. 26. Other leno weave structures can be constructed that are different from the pure leno weave shown in FIG. 26. Other examples of leno weave structures that can be used in fabrics for outer skirts and further details of the structure of the outer fabric layer 2202 can be found in U.S. Patent Application Publication No. 2019/0374337 and U.S. Pat. No. 11,013,600, which are incorporated herein by reference.

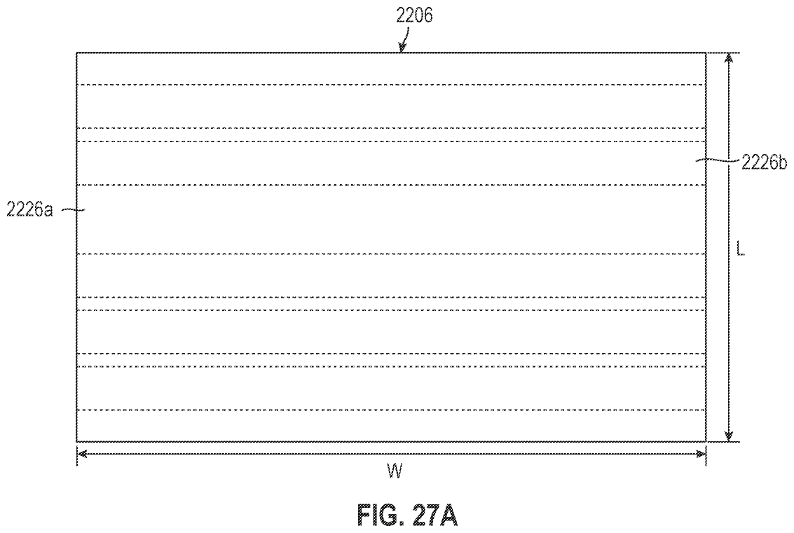

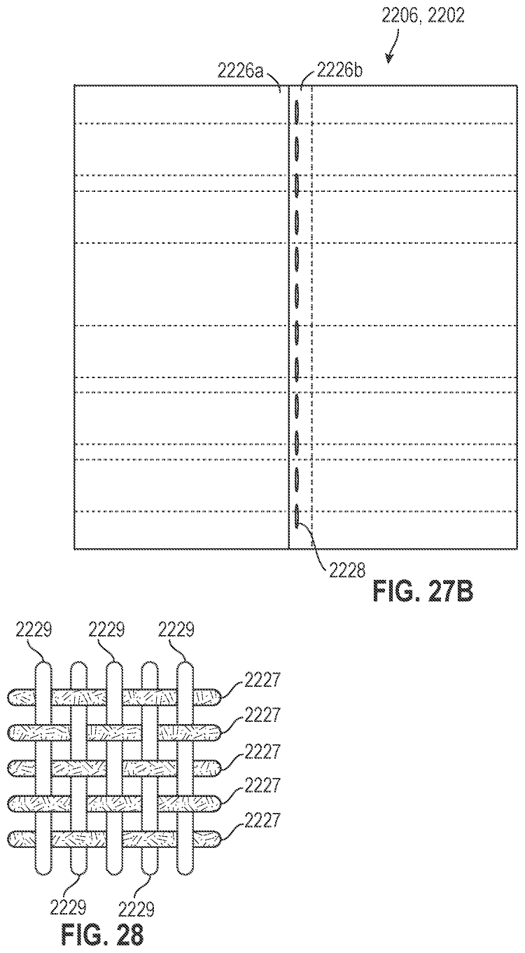

In one example, the woven fabric 2206 can be provided with a width W and length L in an unrolled or flattened configuration (prior to mounting on a frame of a prosthetic valve), as illustrated in FIG. 27A (the structures of the woven fabric 2206 are not shown in FIG. 27A for simplicity). For the woven structure and floating yarn orientations illustrated in FIGS. 24A and 24B, the width W can be along the longitudinal grain of the woven fabric (y-axis in FIG. 24A), and the length L can be along the crosswise grain of the woven fabric (x-axis in FIG. 24A). In the finished tube formed with the woven fabric 2206, the length L can be along the longitudinal direction of the tube, and the width W can be along the circumferential direction of the tube. The width W and length L can be selected based on the desired dimensions of the outer skirt, which can be based on the dimensions of the prosthetic heart valve in the radially compressed configuration and radially expanded configuration. The width W and length L can be additionally selected based on the material of the fabric (e.g., how much stretch the fabric has along the longitudinal and crosswise grains). The woven fabric 2206 can be folded such that longitudinal side edge portions 2226a, 2226b of the fabric overlap each other, as illustrated in FIG. 27B. The overlapping side edge portions 2226a, 2226b can be fastened together (e.g., by sutures 2228) to form a tube corresponding to the first fabric layer of the outer skirt.

In one example, the inner fabric layer 2204 can be constructed from a fabric having a woven structure (e.g., plain weave, including variants). Plain weave (also called tabby weave) is a weave in which every weft yarn alternately passes over and under the warp yarns, forming a crisscross pattern. The warp yarns lie parallel to each other across the warp. FIG. 28 illustrates a plain weave including weft yarns 2227 crisscrossing warp yarns 2229. Plain weave produces the greatest number of intersections per unit space compared to other weave patterns, which can result in a strong and durable fabric. Variations of the plain weave include the rib weave, with either warp or weft yarns heavier, and the basket weave, in which two or more weft yarns pass alternately over and under two or more warp yarns. The high number of intersections in the plain weave can allow the inner fabric layer 2204 to function as an effective barrier between the outer fabric layer 2202 floating structures and the frame of the prosthetic valve. The high number of intersections can also allow the fabric to be relatively thin while being strong, allowing the outer skirt 2200 to be sutured to the frame 402 and allowing the prosthetic heart valve to be crimped without tearing the inner fabric layer 2204. The inner fabric layer 2204 is also designed to provide additional structure to the outer fabric layer 2202 to aid in distributing the load of the frame cells against the native anatomy. The inner fabric layer 2204 can be thinner than the outer fabric layer 2202. In one example, the thickness of the inner fabric layer 2204 can be approximately 50 microns, and the thickness of the outer fabric layer 2202 can be approximately 750 microns.

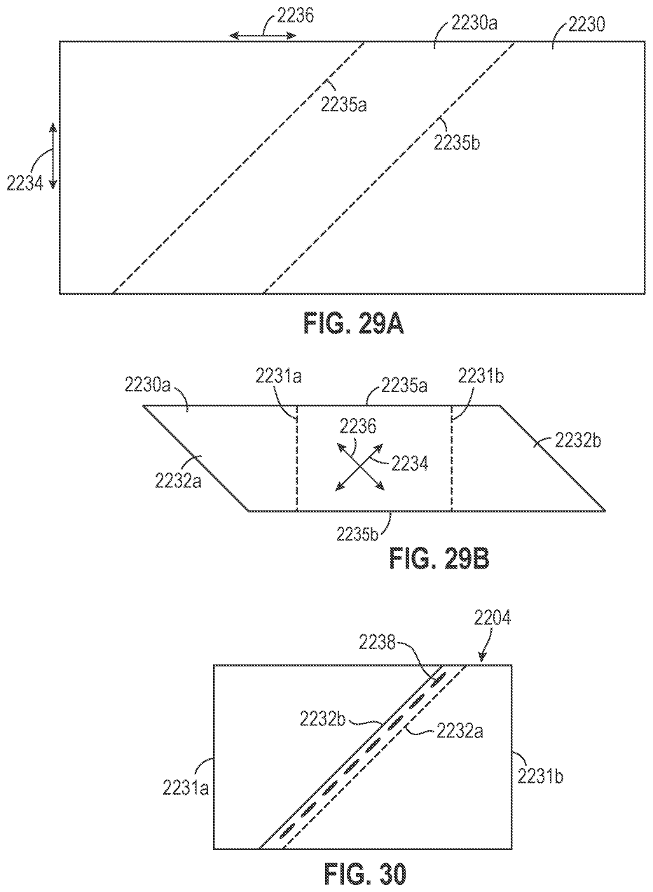

In one example, the inner fabric layer 2204 can be made by providing a fabric 2230, as illustrated in FIG. 29A. A strip 2230a of the fabric 2230 can be cut along bias lines 2235a, 2235b (e.g., lines that are at 45 degrees to the longitudinal grain 2234 and the crosswise grain 2236 of the fabric 2230). The fabric 2230 has a high stretch along the bias compared to along the longitudinal grain 2234 and crosswise grain 2236. By cutting the fabric strip 2230a along the bias of the fabric 2230 and orienting the fabric strip 2230a such that the cut bias edges 2235a, 2235b become the upper and bottom edges, respectively, of the inner fabric layer 2204, as shown in FIG. 29B, the fibers or yarns of the inner fabric layer 2204 extend at an angle between 0 and 90 degrees relative to the upper and lower edges 2235a, 2235b of inner fabric layer 2204 and the central longitudinal axis of the frame of the prosthetic valve. Alternatively, the inner fabric layer 2204 can be woven such that the warp and weft fibers or yarns extend at an angle between 0 and 90 degrees relative to upper and lower edges of the fabric. Desirably, the warp and weft fibers of layer 2204 extend at 45 degree angles relative to the upper and lower edges 2235a, 2235b of the layer 2204 and the central longitudinal axis of the frame of the prosthetic valve. This allows a finished inner fabric layer 2204 to have a relative high stretch in the axial direction when the frame of the prosthetic valve is radially compressed for delivery into a patient. The fabric strip 2230a can be folded (e.g., along lines 2231a, 2231b) to overlap the side edge portions 2236a, 2236b, as illustrated in FIG. 30. The overlapping side edge portions 2236a, 2236b can be fastened together (e.g., by sutures 2238) to form a tube corresponding to the inner fabric layer 2204. In some cases, stabilizing tape can be attached to the overlapping side edge portions 2236a, 2236b. In this case, the overlapping side edge portions 2236a, 2236b and the stabilizing tape can be fastened together (e.g., by sutures) to form the tube.

In another example, the fabric 2230 can be woven in tube form (e.g., using double weaving) with the desired diameter for the inner fabric layer 2204. A desired length of the woven tube can be cut to provide the inner fabric layer 2204.

The fabric 2206 for the outer fabric layer 2202 and the fabric 2230 for the inner fabric layer 2204 can be made from any of various biocompatible thermoplastic polymers, such as polyethylene terephthalate (PET), expanded polytetrafluoroethylene (ePTFE), and Nylon, or other suitable synthetic or natural fibers. In a particular example, both fabrics 2206 and 2230 are made with PET fibers. In some examples, the inner fiber layer 2204 can be substantially porous or have a pore structure that discourages cellular ingrowth, as described for the hermetic layer(s) herein.

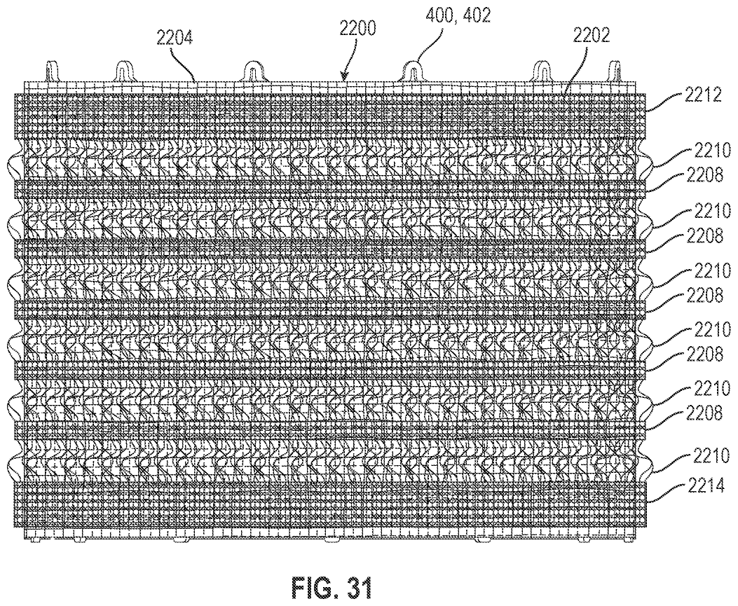

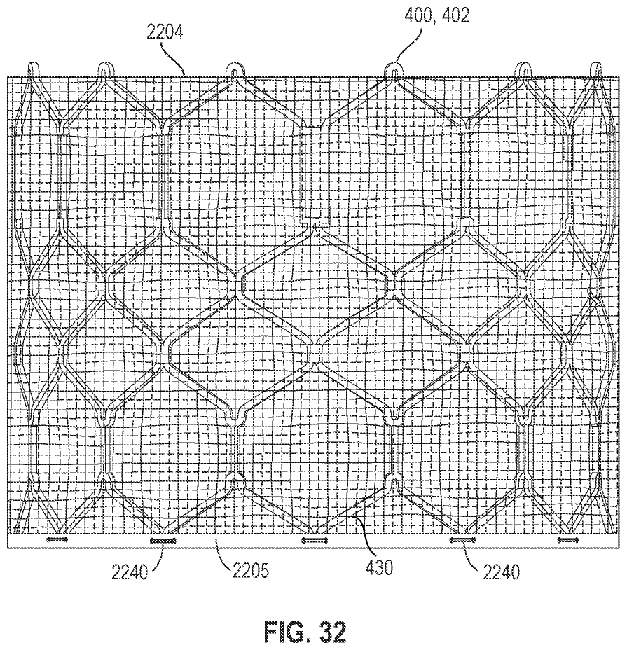

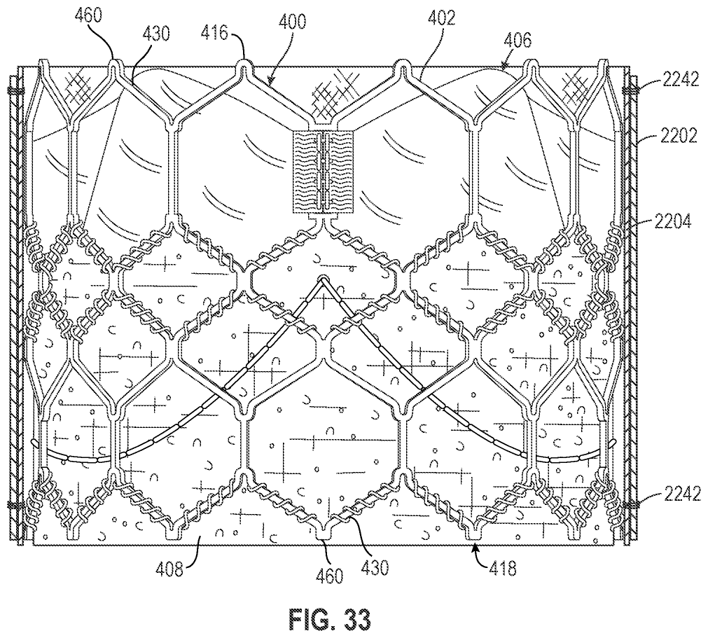

FIG. 31 shows the outer skirt 2200 wrapped around the previously described prosthetic heart valve 400 (as an example), with the prosthetic heart valve 400 in a radially expanded configuration. The outer skirt 2200 is assembled onto the frame 402 of the prosthetic heart valve 400 such that the inner fabric layer 2204 extends around the outer circumferential surface of the frame 402 (shown more clearly in FIG. 32) and the outer fabric layer 2202 extends around the inner fabric layer 2204. The inner fabric layer 2204 in its position between the outer fabric layer 2202 and the frame 402 (see FIG. 33) separates at least a portion of the outer fabric layer 2202 from the frame 402. In one example, the inner fabric layer 2204 is sized and positioned such that the inner fabric layer 2204 isolates at least the second fabric sections 2210 of the outer fabric layer 2202 including the floating structures from the frame 402.

The floating structures (e.g., floating yarns) of the second fabric sections 2210 of the outer fabric layer 2202 provide a cushion that can prevent trauma to the surrounding tissue after implanting the prosthetic heart valve. To optimize the cushioning effect of the cushioning layer, the floating structures preferably project radially outward so that the tissue can contact the floating structures. However, the floating structures are compressible and easily manipulated. When the prosthetic valve is implanted, in the absence of the inner fabric layer 2204, the floating structures can become compressed against the struts 430 of the frame 402 and junctions formed between the struts and some portions of the floating structures can protrude into the frame 402, where they would not less effective in providing the cushion. This phenomenon can occur when the prosthetic valve is implanted within a relatively rigid docking device, such as docking device 1900. In some cases, the docking device, which exerts an inwardly directed force against the outer skirt, can cause permanent deformation of the floating structures. To prevent this scenario, the inner fabric layer 2204 is provided as a separation layer between the frame 402 and the outer fabric layer 2202. The inner fabric layer 2204 can prevent direct contact between the outer fabric layer 2202 and the relatively rigid metal surface of the frame 402, thereby maintaining the resiliency of the floating structures. The inner fabric layer 2204 can also maintain the floating structures on the outside of the frame 402 to maximize contact between the floating structures and the surrounding native tissue.

The outer skirt 2200 is configured to allow the frame 402 to move between the radially compressed and radially expanded configurations. In the illustrated example, the first fabric sections 2208 of the outer fabric layer 2202 are disposed circumferentially around the frame 402, and the second fabric sections 2210 of the outer fabric layer 2202 are disposed circumferentially around the frame 402. The first fabric sections 2208 can expand (e.g., stretch or lengthen) in a circumferential direction of the outer skirt 2200/frame 402 (e.g., due to the elasticity of the leno structures in this direction) when the frame 402 is expanded from a radially compressed configuration to a radially expanded configuration. Expansion of the first fabric sections 2208 in the circumferential direction will increase the diameter of the outer fabric layer 2202, allowing the outer fabric layer 2202 to accommodate the expanding frame. The second fabric sections 2210 can contract in an axial direction of the outer skirt 2200/frame 402 (e.g., due to kinking and twisting of the floating yarns in these portions) when the frame 402 is expanded from the radially compressed configuration to the radially expanded configuration.

The inner fabric layer 2204 and outer fabric layer 2202 can extend substantially along the axial length of the frame 402 so as to substantially cover the outer circumferential surface of the frame 402, as illustrated in FIGS. 31-33. In other examples, the inner fabric layer 2204 and outer fabric layer 2202 may extend only partway along the axial length of the frame 402 (e.g., from the inflow end 418 to about midway of the axial length of the frame 402) to cover a portion of the outer circumferential surface of the frame 402. In some cases, the inner fabric layer 2204 can be longer than the outer fabric layer 2202; and the inner fabric layer 2204 can extend substantially along the axial length of the frame 402 while the outer fabric layer 2202 extends partway along the axial length of the inner fabric layer 2204.

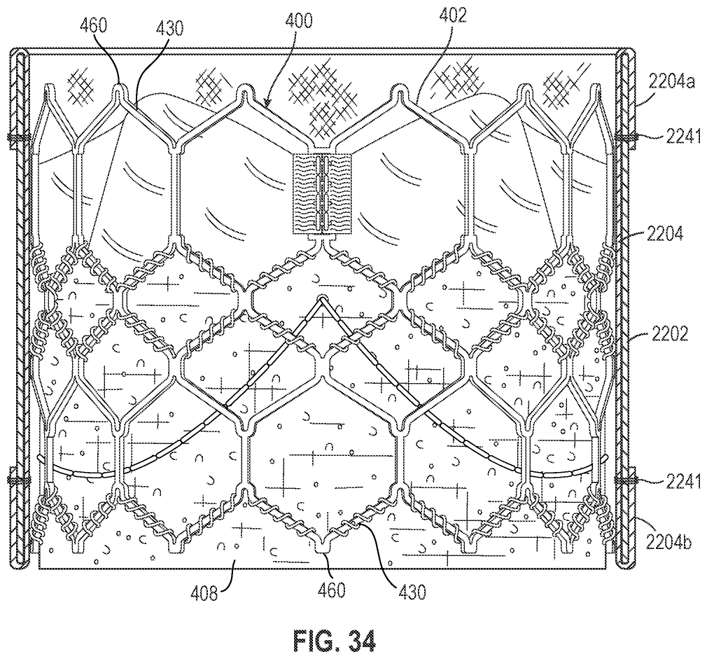

In another example, as illustrated in FIG. 34, the inner fabric layer 2204 can have excess material (or flaps) 2204a, 2204b at the ends that can be folded over the end sections of the outer fabric layer 2202. The flaps can be secured to the outer fabric layer 2202 and the portion of the inner fabric layer 2204 between the outer fabric layer 2202 and the frame 404 (e.g., by sutures 2241 or adhesive). The flaps can prevent direct contact between the apices 460 formed by the struts 430 of the frame 402 at the inflow end 418 and outflow end 416 of the frame 402 and the surrounding tissue.

The outer skirt 2200 can be coupled to the frame 402 using a variety of methods. In one example, the outer fabric layer 2202 can be fastened to the inner fabric layer 2204, and the inner fabric layer 2204 can be fastened to selected struts 430 of the frame 402 (e.g., by sutures 2240 as illustrated in FIG. 32). A reinforcement material 2205 may be used at the base of the inner fabric layer 2204 attached to apices 460 at the inflow end 418 of the frame 402. In another example, the outer fabric layer 2202 can be fastened to the inner fabric layer 2204, and the inner fabric layer 2204 can be fastened to an inner skirt 408 disposed within the prosthetic heart valve. Other examples of coupling an outer skirt to a prosthetic heart valve disclosed in other implementations herein can be applied to coupling the outer skirt 2200 to a prosthetic heart valve.

Although the outer skirt 2200 has been illustrated as covering the frame 402 of the prosthetic heart valve 400, it should be understood that the outer skirt 2200 could be applied to any of the other prosthetic heart valves disclosed herein. In addition, the outer skirt 2200 can be used with or without an inner skirt disposed within the frame of the prosthetic heart valve. In cases where the outer skirt 2200 is used without an inner skirt within the prosthetic heart valve or where the inner skirt within the prosthetic heart valve is not hermetic, the inner fabric layer 2204 can be hermetic.

### Additional Examples

Additional examples based on principles described herein are enumerated below. Further examples falling within the scope of the subject can be configured by, for example, taking one feature of an example in isolation, taking more than one feature of an example in combination, or combining one or more features of one example with one or more features of one or more other examples.

Example 1—A prosthetic heart valve comprises an annular frame that is radially collapsible and expandable between a radially-compressed configuration and a radially-expanded configuration, the annular frame having an inflow end and an outflow end separated from the inflow end along an axial direction of the frame; a valvular structure supported within the annular frame and comprising a plurality of leaflets, each leaflet having a cusp edge portion and tabs on opposite sides with respect to a centerline of the leaflet, the cusp edge portion being curved along at least a portion thereof to form an apex at the centerline of the leaflet, the valvular structure being coupled to the frame via a plurality of commissure assemblies formed by paired tabs of adjacent leaflets; and an inner skirt disposed on a radially-inner circumferential surface of the annular frame and coupled thereto, the inner skirt comprising a hermetic layer constructed such that, when the prosthetic heart valve is implanted in a patient, ingrowth of cells from surrounding native tissue of the patient into the hermetic layer is prevented. The inner skirt is disposed between the annular frame and the cusp edge portion of each leaflet along a radial direction of the annular frame, and the inner skirt extends along the axial direction of the frame from at least the apices of the cusp edge portions of the leaflets to at least the plurality of commissure assemblies.

Example 2—The prosthetic heart valve according to any example herein, particularly example 1, wherein the hermetic layer is substantially nonporous or has pores therein that are sized to discourage cellular ingrowth.

Example 3—The prosthetic heart valve according to any example herein, particularly any one of examples 1 and 2, wherein the hermetic layer is formed of a hydrophobic polymer material comprising polytetrafluoroethylene (PTFE), expanded PTFE (ePTFE), urethane, polyurethane (PU), thermoplastic PU (TPU), silicone, or combinations or copolymers thereof.

Example 4—The prosthetic heart valve according to any example herein, particularly any one of examples 1-3, wherein the cusp edge portion of each leaflet is attached to the inner skirt.

Example 5—The prosthetic heart valve according to any example herein, particularly example 4, wherein the cusp edge portion of each leaflet is attached to the inner skirt via one or more sutures.

Example 6—The prosthetic heart valve according to any example herein, particularly any one of examples 4-5, wherein the inner skirt further comprises a scrim layer disposed in a region along the axial direction where the cusp edge portions attach to the inner skirt.

Example 7—The prosthetic heart valve according to any example herein, particularly any one of examples 1-6, wherein the inner skirt substantially covers an entirety of the radially-inner circumferential surface of the annular frame between the inflow and outflow ends.

Example 8—The prosthetic heart valve according to any example herein, particularly any one of examples 1-7, wherein the commissure assemblies extend radially through respective openings in the inner skirt and through respective commissure windows of the annular frame.

Example 9—The prosthetic heart valve according to any example herein, particularly any one of examples 1-8, further comprising one or more protective covers, each protective cover comprising a second hermetic layer of hydrophobic polymer material, the second hermetic layer being substantially nonporous or having pores therein that are sized to discourage cellular ingrowth, the one or more protective covers being disposed on respective radially-outer circumferential surface portions of the annular frame where the commissure assemblies extend through the commissure windows.

Example 10—The prosthetic heart valve according to any example herein, particularly example 9, wherein the one or more protective covers is a single annular cover that wraps around a portion of a radially-outer circumferential surface of the annular frame.

Example 11—The prosthetic heart valve according to any example herein, particularly any one of examples 1-8, wherein the inner skirt is coupled to the frame, and the commissure assemblies extend radially to respective radially-inner surface portions of the inner skirt and are coupled thereto by one or more sutures, thereby indirectly coupling the valvular structure to the frame.

Example 12—The prosthetic heart valve according to any example herein, particularly any one of examples 1-11, wherein the hermetic layer comprises a lamination of sublayers.

Example 13—The prosthetic heart valve according to any example herein, particularly any one of examples 1-12, wherein the hermetic layer comprises a layer formed directly on the radially-inner circumferential surface of the annular frame.

Example 14—The prosthetic heart valve according to any example herein, particularly example 13, wherein the hermetic layer comprises an electrospun, dip-coated, or spray-coated layer on the radially-inner circumferential surface of the annular frame.

Example 15—The prosthetic heart valve according to any example herein, particularly any one of examples 13-14, wherein the hermetic layer is formed on the frame so as to be coupled thereto without sutures.

Example 16—The prosthetic heart valve according to any example herein, particularly any one of examples 1-12, wherein the hermetic layer comprises a layer formed separate from the frame and subsequently attached to the frame.

Example 17—The prosthetic heart valve according to any example herein, particularly example 16, wherein the hermetic layer is an extruded layer or cast layer attached to the annular frame by one or more sutures.

Example 18—The prosthetic heart valve according to any example herein, particularly any one of examples 1-17, further comprising an outer skirt disposed over a portion of a radially-outer circumferential surface of the annular frame, the outer skirt extending along the axial direction from the inflow end of the frame.

Example 19—The prosthetic heart valve according to any example herein, particularly example 18, wherein the outer skirt is coupled to struts of the annular frame, the inner skirt, or combinations thereof.

Example 20—The prosthetic heart valve according to any example herein, particularly any one of examples 18-19, wherein the outer skirt is coupled to the annular frame or the inner skirt by one or more sutures.

Example 21—The prosthetic heart valve according to any example herein, particularly any one of examples 18-20, wherein the outer skirt is disposed on the radially-outer circumferential surface portion of the annular frame and is coupled thereto, the outer skirt comprising a third hermetic layer of hydrophobic polymer material, the third hermetic layer being substantially nonporous or having pores therein that are sized to discourage cellular ingrowth.

Example 22—The prosthetic heart valve according to any example herein, particularly any one of examples 18-21, wherein the outer skirt extends along the axial direction from at least the inflow end of the frame to at least the plurality of commissure assemblies.

Example 23—The prosthetic heart valve according to any example herein, particularly any one of examples 18-22, wherein the outer skirt substantially covers an entirety of the radially-outer circumferential surface of the annular frame between the inflow and outflow ends.

Example 24—The prosthetic heart valve according to any example herein, particularly any one of examples 21-23, wherein the inner and outer skirts are part of a same unitary skirt structure that wraps around the inflow end of the annular frame, the hermetic layer and the third hermetic layer being a same hermetic layer.

Example 25—The prosthetic heart valve according to any example herein, particularly any one of examples 18-23, wherein a portion of the outer skirt faces or overlaps a portion of the inner skirt at the inflow end of the annular frame and is coupled to said portion of the inner skirt.

Example 26—The prosthetic heart valve according to any example herein, particularly any one of examples 18-25, wherein the third hermetic layer comprises a lamination of sublayers.

Example 27—The prosthetic heart valve according to any example herein, particularly any one of examples 18-26, wherein the third hermetic layer comprises a layer formed directly on the radially-outer circumferential surface of the annular frame.

Example 28—The prosthetic heart valve according to any example herein, particularly example 27, wherein the third hermetic layer comprises an electrospun, dip-coated, or spray-coated layer on the radially-outer circumferential surface of the annular frame.

Example 29—The prosthetic heart valve according to any example herein, particularly any one of examples 27-28, wherein the third hermetic layer is formed on the frame so as to be coupled thereto without sutures.

Example 30—The prosthetic heart valve according to any example herein, particularly any one of examples 18-26, wherein the third hermetic layer comprises a layer formed separate from the frame and subsequently attached to the frame.

Example 31—The prosthetic heart valve according to any example herein, particularly example 30, wherein the third hermetic layer is an extruded or cast layer attached to the annular frame by one or more sutures.

Example 32—The prosthetic heart valve according to any example herein, particularly any one of examples 18-20, wherein the outer skirt comprises polyethylene terephthalate (PET).

Example 33—The prosthetic heart valve according to any example herein, particularly any one of examples 1-32, wherein, for each commissure assembly, the tabs thereof are separated and folded to form a T-shape, such that each tab comprises a first portion that extends along a circumferential direction of the frame and contacts a coupling member, and a second portion that extends along a radial direction of the frame, contacts the corresponding second portion of the other tab of the pair, and connects the first portion to a central portion of the leaflet.

Example 34—The prosthetic heart valve according to any example herein, particularly example 33, wherein the coupling member comprises a flexible cloth or fabric.

Example 35—The prosthetic heart valve according to any example herein, particularly any one of examples 33-34, wherein the coupling member comprises a fourth hermetic layer of hydrophobic polymer material, the fourth hermetic layer being substantially nonporous or having pores therein that are sized to discourage cellular ingrowth.

Example 36—A prosthetic heart valve comprises an annular frame that is radially collapsible and expandable between a radially-compressed configuration and a radially-expanded configuration, the annular frame having an inflow end and an outflow end separated from the inflow end along an axial direction of the frame; and a valvular structure supported within the annular frame and comprising a plurality of leaflets, each leaflet having a cusp edge portion and tabs on opposite sides with respect to a centerline of the leaflet, the cusp edge portion being curved along at least a portion thereof to form an apex at the centerline of the leaflet, the valvular structure being coupled to the frame via a plurality of commissure assemblies formed by paired tabs of adjacent leaflets. The annular frame is encapsulated by a hermetic layer constructed such that, when the prosthetic heart valve is implanted in a patient, ingrowth of cells from surrounding native tissue of the patient into the hermetic layer is prevented.

Example 37—The prosthetic heart valve according to any example herein, particularly example 36, wherein the hermetic layer is substantially nonporous or has pores therein that are sized to discourage cellular ingrowth.

Example 38—The prosthetic heart valve according to any example herein, particularly any one of examples 36-37, wherein the hermetic layer is formed of a hydrophobic polymer material comprising polytetrafluoroethylene (PTFE), expanded PTFE (ePTFE), urethane, polyurethane (PU), thermoplastic PU (TPU), silicone, or combinations or copolymers thereof.

Example 39—The prosthetic heart valve according to any example herein, particularly any one of examples 36-38, wherein the cusp edge portion of each leaflet is attached to the hermetic layer.

Example 40—The prosthetic heart valve according to any example herein, particularly example 39, wherein the cusp edge portion of each leaflet is attached to the hermetic layer via one or more sutures.

Example 41—The prosthetic heart valve according to any example herein, particularly any one of examples 39-40, comprising a scrim layer disposed between the annular frame and the encapsulating hermetic layer, the scrim layer being in a region along the axial direction where the cusp edge portions attach to the hermetic layer.

Example 42—The prosthetic heart valve according to any example herein, particularly any one of examples 36-41, wherein the commissure assemblies extend radially through respective openings in the encapsulating hermetic layer and through respective commissure windows of the annular frame, and the commissure assemblies are coupled to the commissure windows by one or more sutures.

Example 43—The prosthetic heart valve according to any example herein, particularly example 42, further comprising one or more protective covers, each protective cover comprising a second hermetic layer of hydrophobic polymer material, the second hermetic layer being substantially nonporous or having pores therein that are sized to discourage cellular ingrowth, the one or more protective covers being disposed on respective radially-outer circumferential surface portions of the annular frame where the commissure assemblies extend through the commissure windows.

Example 44—The prosthetic heart valve according to any example herein, particularly example 43, wherein the one or more protective covers is a single annular cover that wraps around a portion of a radially-outer circumferential surface of the annular frame.

Example 45—The prosthetic heart valve according to any example herein, particularly any one of examples 36-44, wherein the commissure assemblies extend radially to respective radially-inner surface portions of the encapsulating hermetic layer and are coupled thereto by one or more sutures, thereby coupling the valvular structure to the frame.

Example 46—The prosthetic heart valve according to any example herein, particularly any one of examples 36-45, wherein the hermetic layer comprises a lamination of sublayers.

Example 47—The prosthetic heart valve according to any example herein, particularly any one of examples 36-46, wherein the hermetic layer comprises an electrospun, dip-coated, or spray-coated layer.

Example 48—The prosthetic heart valve according to any example herein, particularly any one of examples 36-47, wherein, for each commissure assembly, the tabs thereof are separated and folded to form a T-shape, such that each tab comprises a first portion that extends along a circumferential direction of the frame and contacts a coupling member, and a second portion that extends along a radial direction of the frame, contacts the corresponding second portion of the other tab of the pair, and connects the first portion to a central portion of the leaflet.

Example 49—The prosthetic heart valve according to any example herein, particularly example 48, wherein the coupling member comprises a flexible cloth or fabric.

Example 50—The prosthetic heart valve according to any example herein, particularly any one of examples 48-49, wherein the coupling member comprises a third hermetic layer of hydrophobic polymer material, the third hermetic layer being substantially nonporous or having pores therein that are sized to discourage cellular ingrowth.

Example 51—A prosthetic heart valve comprising a frame; a valvular structure coupled to the frame and comprising a plurality of leaflets; and means for preventing ingrowth of cells from surrounding native tissue of a patient onto leaflets of the valvular structure.

Example 52—The prosthetic heart valve according to any example herein, particularly example 51, wherein the means for preventing ingrowth of cells comprises one or more hermetic layers arranged to be disposed between the valvular structure and surrounding native tissue when the prosthetic heart valve is implanted in the patient.

Example 53—The prosthetic heart valve according to any example herein, particularly example 52, wherein each hermetic layer comprises a hydrophobic polymer material, and is substantially nonporous or has pores therein that are sized to discourage cellular ingrowth.

Example 54—The prosthetic heart valve according to any example herein, particularly example 53, wherein the hydrophobic polymer material comprises polytetrafluoroethylene (PTFE), expanded PTFE (ePTFE), urethane, polyurethane (PU), thermoplastic PU (TPU), silicone, or combinations or copolymers thereof.

Example 55—The prosthetic heart valve according to any example herein, particularly any one of examples 1-54, wherein the valvular structure is a bicuspid structure with two leaflets and two commissure assemblies, and the valvular structure is coupled to the frame via the commissure assemblies on diametrically opposite sides of the frame from each other.

Example 56—The prosthetic heart valve according to any example herein, particularly any one of examples 1-54, wherein the valvular structure is a tricuspid structure with three leaflets and three commissure assemblies, and the valvular structure is coupled to the frame via the three commissure assemblies equally spaced along a circumferential direction of the frame.

Example 57—The prosthetic heart valve according to any example herein, particularly any one of examples 1-56, wherein the frame is formed of a plastically-expandable material or a self-expanding material.

Example 58—The prosthetic heart valve according to any example herein, particularly any one of examples 1-57, wherein the prosthetic heart valve is constructed for implantation in an existing heart valve within a patient.

Example 59—The prosthetic heart valve according to any example herein, particularly any one of examples 1-58, wherein the prosthetic heart valve is constructed for implantation at an aortic position or a mitral position.

Example 60—An assembly comprising a delivery apparatus comprising an elongated shaft; and the prosthetic heart valve of any one of examples 1-59 mounted on the elongated shaft in the radially-compressed configuration for delivery into a patient's body.

Example 61—A method of implanting a prosthetic heart valve in a patient's body comprises inserting a distal end of a delivery apparatus into vasculature of a patient, the delivery apparatus comprising an elongated shaft, the prosthetic heart valve of any one of claims 1-59 being releasably mounted in the radially-compressed configuration on the elongated shaft of the delivery apparatus; advancing the prosthetic heart valve to a desired implantation site; and using the delivery apparatus to expand the prosthetic heart valve to the radially-expanded configuration, thereby implanting the prosthetic heart valve at the desired implantation site.

Example 62—A method of implanting a prosthetic heart valve in a patient's body comprises inserting a distal end of a delivery apparatus into vasculature of a patient, the delivery apparatus comprising an elongated shaft, the prosthetic heart valve of any one of examples 1-59 being releasably mounted in the radially-compressed configuration on the elongated shaft of the delivery apparatus; advancing the prosthetic heart valve to a desired implantation site; and deploying the prosthetic heart valve from the delivery apparatus such that the prosthetic heart valve self-expands to the radially-expanded configuration, thereby implanting the prosthetic heart valve at the desired implantation site.

Example 63—The method of any example herein, particularly any one of examples 61-62, further comprising installing a valve dock at the desired implantation site, wherein the prosthetic heart valve in the radially-expanded configuration is mounted within the valve dock.

Example 64—The method of any example herein, particularly any one of examples 61-63, wherein the advancing to the desired implantation site employs transfemoral, transventricular, transapical, or transseptal approaches.

Example 65—A method of assembling a prosthetic heart valve having a plurality of leaflets comprises providing an inner skirt on a radially-inner circumferential surface of an annular frame, the annular frame being radially collapsible and expandable between a radially-compressed configuration and a radially-expanded configuration, the annular frame having an inflow end and an outflow end separated from the inflow end along an axial direction of the frame, the inner skirt comprising a hermetic layer, the hermetic layer comprising a layer formed directly on the radially-inner circumferential surface of the annular frame; forming a plurality of commissure assemblies with the plurality of leaflets, each leaflet having a cusp edge portion and tabs on opposite sides with respect to a centerline of the leaflet, the cusp edge portion being curved along at least a portion thereof to form an apex at the centerline of the leaflet, each commissure assembly formed by paired tabs of adjacent leaflets; and coupling each commissure assembly to the annular frame, the inner skirt being disposed between the annular frame and the cusp edge portion of each leaflet along a radial direction of the annular frame, and the inner skirt extending along the axial direction of the frame from at least the apices of the cusp edge portions of the leaflets to at least the plurality of commissure assemblies, wherein the hermetic layer is constructed such that, when the prosthetic heart valve is implanted in a patient, ingrowth of cells from surrounding native tissue of the patient into the hermetic layer is prevented.

Example 66—The method of any example herein, particularly example 65, wherein the providing the inner skirt comprises electrospinning, dip-coating, or spray-coating the hermetic layer onto the radially-inner circumferential surface of the annular frame.

Example 67—The method of any example herein, particularly any one of examples 65-66, wherein the hermetic layer is formed on the annular frame so as to be coupled thereto without sutures.

Example 68—The method of any example herein, particularly any one of examples 65-67, further comprises providing an outer skirt on a portion of a radially-outer circumferential surface of the annular frame, the outer skirt comprising a second hermetic layer of hydrophobic polymer material, the second hermetic layer being substantially nonporous or having pores therein that are sized to discourage cellular ingrowth.

Example 69—The method of any example herein, particularly example 68, wherein the providing the outer skirt comprises electrospinning, dip-coating, or spray-coating the second hermetic layer onto the radially-outer circumferential surface of the annular frame.

Example 70—The method of any example herein, wherein the providing the outer skirt comprises coupling the outer skirt to the radially-outer circumferential surface portion of the annular frame using one or more sutures.

Example 71—The method of any example herein, particularly example 70, wherein the second hermetic layer of the outer skirt is formed by extruding or casting.

Example 72—A method of assembling a prosthetic heart valve having a plurality of leaflets comprises coupling an inner skirt to a radially-inner circumferential surface of an annular frame, the annular frame being radially collapsible and expandable between a radially-compressed configuration and a radially-expanded configuration, the annular frame having an inflow end and an outflow end separated from the inflow end along an axial direction of the frame, the inner skirt comprising a hermetic layer; forming a plurality of commissure assemblies with the plurality of leaflets, each leaflet having a cusp edge portion and tabs on opposite sides with respect to a centerline of the leaflet, the cusp edge portion being curved along at least a portion thereof to form an apex at the centerline of the leaflet, each commissure assembly formed by paired tabs of adjacent leaflets; and coupling each commissure assembly to the annular frame, the inner skirt being disposed between the annular frame and the cusp edge portion of each leaflet along a radial direction of the annular frame, and the inner skirt extending along the axial direction of the frame from at least the apices of the cusp edge portions of the leaflets to at least the plurality of commissure assemblies, wherein the hermetic layer is constructed such that, when the prosthetic heart valve is implanted in a patient, ingrowth of cells from surrounding native tissue of the patient into the hermetic layer is prevented.

Example 73—The method of any example herein, particularly example 72, wherein, prior to the coupling the inner skirt to the annular frame, forming the hermetic layer of the inner skirt by extrusion or casting.

Example 74—The method of any example herein, particularly example 73, wherein the coupling the inner skirt to the annular frame is via one or more sutures.

Example 75—The method of any example herein, particularly any of examples 72-74, further comprising providing an outer skirt on a portion of a radially-outer circumferential surface of the annular frame, the outer skirt comprising a second hermetic layer of hydrophobic polymer material, the second hermetic layer being substantially nonporous or having pores therein that are sized to discourage cellular ingrowth.

Example 76—The method of any example herein, particularly example 75, wherein the providing the outer skirt comprises electrospinning, dip-coating, or spray-coating the second hermetic layer onto the radially-outer circumferential surface of the annular frame.

Example 77—The method of any example herein, particularly example 75, wherein the providing the outer skirt comprises coupling the outer skirt to the radially-outer circumferential surface portion of the annular frame using one or more sutures.

Example 78—The method of any example herein, particularly example 77, wherein the second hermetic layer of the outer skirt is formed by extruding or casting.

Example 79—The method of any example herein, particularly any of examples 72-78, wherein the hermetic layer is an extruded or cast layer.

Example 80—The method of any example herein, particularly any of examples 72-79, wherein the coupling the inner skirt to the annular frame comprises using one or more sutures to attach the hermetic layer to struts of the annular frame.

Example 81—The method of any example herein, particularly any of examples 65-79, further comprising coupling the cusp edge portion of each leaflet to the inner skirt using one or more sutures.

Example 82—The method of any example herein, particularly example 81, wherein the coupling the inner skirt to a radially-inner circumferential surface of the annular frame is such that a scrim layer of the inner skirt is disposed in a region along the axial direction where the cusp edge portions attach to the inner skirt.

Example 83—The method of any example herein, particularly any of examples 65-82, wherein the inner skirt substantially covers an entirety of the radially-inner circumferential surface of the annular frame between the inflow and outflow ends.

Example 84—The method of any example herein, particularly any of examples 65-83, wherein the hermetic layer is substantially nonporous or has pores therein that are sized to discourage cellular ingrowth; and/or the hermetic layer is formed of a hydrophobic polymer material.

Example 85—The method of any example herein, particularly any one of examples 65-84, can further comprise forming openings in the inner skirt at locations corresponding to commissure windows of the annular frame, and the coupling each commissure assembly to the annular frame comprises inserting the tabs of the commissure assembly through one of the opening in the inner skirt and through one of the commissure windows, such that first portions of the tabs are disposed on a radially-outer side of the annular frame; separating and folding the first portions of the tabs such that the first portions extend away from each other along a circumferential direction of the annular frame; and using one or more sutures to attach the first portions of the tabs to the respective commissure window, to portions of the leaflets on a radially-inner side of the commissure window, or to any combination thereof.

Example 86—The method of any example herein, particularly example 85, can further comprise coupling one or more protective covers over the first portions of the tabs on the radially-outer side of the annular frame, each protective cover comprising a third hermetic layer of hydrophobic polymer material, the third hermetic layer being substantially nonporous or having pores therein that are sized to discourage cellular ingrowth.

Example 87—The method of any example herein, particularly example 86, wherein the one or more protective covers is a single annular cover that wraps around a radially-outer circumferential surface portion of the annular frame.

Example 88—The method of any example herein, particularly any one of examples 65-84, wherein the coupling each commissure assembly to the annular frame comprises coupling each commissure assembly to the inner skirt.

Example 89—A method of assembling a prosthetic heart valve having a plurality of leaflets comprises encapsulating an annular frame with a hermetic layer, the annular frame being radially collapsible and expandable between a radially-compressed configuration and a radially-expanded configuration, the annular frame having an inflow end and an outflow end separated from the inflow end along an axial direction of the frame, the hermetic layer being constructed such that, when the prosthetic heart valve is implanted in a patient, ingrowth of cells from surrounding native tissue of the patient into the hermetic layer is prevented; forming a plurality of commissure assemblies with the plurality of leaflets, each leaflet having a cusp edge portion and tabs on opposite sides with respect to a centerline of the leaflet, the cusp edge portion being curved along at least a portion thereof to form an apex at the centerline of the leaflet, each commissure assembly formed by paired tabs of adjacent leaflets; and coupling each commissure assembly to the annular frame.

Example 90—The method of any example herein, particularly example 89, further comprising coupling an outer skirt over a portion of a radially-outer circumferential surface of the annular frame, the outer skirt extending along the axial direction from the inflow end of the frame

Example 91—The method of any example herein, particularly example 90, wherein the outer skirt comprises polyethylene terephthalate (PET).

Example 92—The method of any example herein, particularly any of examples 90-91, wherein the coupling of the outer skirt to the annular frame is via one or more sutures.

Example 93—The method of any example herein, particularly any of examples 90-92, wherein the outer skirt substantially covers an entirety of the radially-outer circumferential surface of the annular frame between the inflow and outflow ends.

Example 94—The method of any example herein, particularly any of examples 89-93, which can further comprise coupling the cusp edge portion of each leaflet to the hermetic layer.

Example 95—The method of any example herein, particularly example 94, wherein the cusp edge portion of each leaflet is attached to the hermetic layer by one or more sutures.

Example 96—The method of any example herein, particularly any of examples 89-95, wherein a scrim layer is disposed in a region along the axial direction where the cusp edge portions attach to the hermetic layer.

Example 97—The method of any example herein, particularly any of examples 89-96, which can further comprise forming openings in the hermetic layer at locations corresponding to commissure windows of the annular frame, and wherein the coupling each commissure assembly to the annular frame comprises inserting the tabs of the commissure assembly through one of the opening in the hermetic layer and through one of the commissure windows, such that first portions of the tabs are disposed on a radially-outer side of the annular frame; separating and folding the first portions of the tabs such that the first portions extend away from each other along a circumferential direction of the annular frame; and using one or more sutures to attach the first portions of the tabs to the respective commissure window, to portions of the leaflets on a radially-inner side of the commissure window, to the hermetic layer, or to any combination thereof.

Example 98—The method of any example herein, particularly example 97, can further comprise coupling one or more protective covers over the first portions of the tabs on the radially-outer side of the annular frame, each protective cover comprising a second hermetic layer of hydrophobic polymer material, the second hermetic layer being substantially nonporous or having pores therein less that are sized to discourage cellular ingrowth.

Example 99—The method of any example herein, particularly example 98, wherein the one or more protective covers is a single annular cover that wraps around a radially-outer circumferential surface portion of the encapsulated annular frame.

Example 100—The method of any example herein, particularly any one of examples 89-99, wherein the coupling each commissure assembly to the annular frame comprises coupling each commissure assembly to the hermetic layer.

Example 101—The method of any example herein, particularly any one of examples 65-100, wherein each hermetic layer, some of the hermetic layers, or one of the hermetic layers comprises a lamination of sublayers.

Example 102—The method of any example herein, particularly any one of examples 65-101, wherein each hermetic layer, some of the hermetic layers, or all of the hermetic layers are substantially nonporous or have pores therein that are sized to discourage cellular ingrowth; and/or each hermetic layer, some of the hermetic layers, or all of the hermetic layers are formed of a hydrophobic polymer material.

Example 103—The method of any example herein, particularly any one of examples 65-102, wherein each hermetic layer, some of the hermetic layers, or one of the hermetic layers comprises polytetrafluoroethylene (PTFE), expanded PTFE (ePTFE), urethane, polyurethane (PU), thermoplastic PU (TPU), silicone, or combinations or copolymers thereof.

Example 104—A leaflet for a valvular structure of a prosthetic heart valve comprises a first portion; first and second tabs on opposite sides of the first portion with respect to a centerline of the first portion, each of the tabs having a base edge and an outer edge, the outer edges of the first and second tabs being substantially parallel to each other; and a second portion having a half-elliptical or semi-elliptical shape that defines a cusp edge, the cusp edge extending from the base edge of the first tab to the base edge of the second tab, wherein the cusp edge is curved along its entire length between the base edges of the first and second tabs.

Example 105—The leaflet of any example herein, particularly example 104, wherein, at the base edge of the first tab, the cusp edge of the second portion has a tangent that is substantially parallel to the outer edge of the first tab, and, at the base edge of the second tab, the cusp edge of the second portion has a tangent that is substantially parallel to the outer edge of the second tab.

Example 106—The leaflet of any example herein, particularly any one of examples 104-105, wherein the first portion defines a first edge that extends between the first and second tabs, and the second portion is on a side of the first portion opposite the first edge.

Example 107—The leaflet of any example herein, particularly any one of examples 104-106, wherein the outer edges of the first and second tabs are parallel to the centerline of the first portion.

Example 108—The leaflet of any example herein, particularly any one of examples 104-107, wherein an apex of the cusp edge of the second portion is along the centerline of the first portion.

Example 109—The leaflet of any example herein, particularly any one of examples 104-108, wherein a major axis of the half-elliptical or semi-elliptical shape is perpendicular to the centerline of the first portion.

Example 110—The leaflet of any example herein, particularly any one of examples 104-109, wherein a major axis of the half-elliptical or semi-elliptical shape is substantially coincident with the base edges of the first and second tabs.

Example 111—The leaflet of any example herein, particularly any one of examples 104-110, wherein a thickness of one or more of the first portion of the leaflet, the second portion of the leaflet, and the tabs is less than or equal to 0.012 inches (305 μm).

Example 112—A prosthetic heart valve comprises an annular frame that is radially collapsible and expandable between a radially-compressed configuration and a radially-expanded configuration, the annular frame having an inflow end and an outflow end separated from the inflow end along an axial direction of the frame; a valvular structure supported within the annular frame and comprising a plurality of leaflets, each leaflet according to any one of examples 104-111, the valvular structure being coupled to the frame via a plurality of commissure assemblies formed by paired ones of the tabs from adjacent leaflets; and an inner skirt disposed on a radially-inner circumferential surface of the annular frame and coupled thereto, a cusp edge portion of the second portion of each leaflet at the respective cusp edge being coupled to the inner skirt, wherein one or more sutures couple the cusp edge portion of each leaflet to the inner skirt, wherein a suture line formed by the one or more sutures follows a curvature of the cusp edge, and wherein the suture line formed by the one or more sutures is continuous from apices of the cusp edges substantially to the commissure assemblies.

Example 113—The prosthetic heart valve of any example herein, particularly example 112, wherein the suture line extends to the base edges of the first and second tabs of each leaflet, or to respective locations that are substantially adjacent to the base edges of the first and second tabs of the leaflets.

Example 114—The prosthetic heart valve of any example herein, particularly any one of examples 112-113, wherein the outer edges of the tabs of the leaflets are substantially parallel to the axial direction of the frame.

Example 115—The prosthetic heart valve of any example herein, particularly any one of examples 112-114, wherein, in an open configuration of the valvular structure, the centerline of the first portion of each leaflet is substantially parallel to the axial direction of the frame.

Example 116—The prosthetic heart valve of any example herein, particularly any one of examples 112-115, wherein, for each commissure assembly, the tabs thereof are separated and folded to form a T-shape, such that each tab has a first part that extends along a circumferential direction of the frame and contacts a coupling member, and a second part that extends along a radial direction of the frame, contacts the corresponding second part of the other tab of the pair, and connects the first part to the first portion of the leaflet.

Example 117—The prosthetic heart valve of any example herein, particularly example 116, wherein the coupling member comprises a flexible cloth or fabric.

Example 118—The prosthetic heart valve of any example herein, particularly any one of examples 112-117, wherein the second portions of adjacent leaflets are only indirectly coupled to each other via the coupling to the inner skirt.

Example 119—A prosthetic heart valve comprises an annular frame having an inflow end and an outflow end separated from the inflow end along an axial direction of the frame; and valvular means for regulating blood flow through the prosthetic heart valve in a hemodynamic situation at an implanted location within a patient where a pressure gradient across the prosthetic heart valve is less than or equal to 30 mmHg.

Example 120—The prosthetic heart valve of any example herein, particularly example 119, wherein the valvular means comprises a valvular structure supported within the annular frame and comprising a plurality of leaflets, the valvular structure being coupled to the frame via a plurality of commissure assemblies formed by paired tabs of adjacent leaflets.

Example 121—The prosthetic heart valve of any example herein, particularly any one of examples 119-120, wherein the valvular means further comprises one or more sutures coupling a cusp edge portion of each leaflet of the valvular structure to an inner skirt attached to the annular frame, a suture line formed by the one or more sutures following a curvature of the cusp edge portion, the suture line being continuous from apices of the cusp edge portion substantially to the commissure assemblies.

Example 122—The prosthetic heart valve of any example herein, particularly any one of examples 119-121, wherein, in an open configuration of the valvular structure, a centerline of each leaflet is substantially parallel to the axial direction of the frame.

Example 123—The prosthetic heart valve of any example herein, particularly any one of examples 112-122, further comprising an outer skirt disposed over a radially-outer circumferential surface portion of the annular frame, the outer skirt extending along the axial direction from the inflow end of the frame.

Example 124—The prosthetic heart valve of any example herein, particularly any one of examples 112-123, wherein the valvular structure is a bicuspid structure with two leaflets and two commissure assemblies, and the valvular structure is coupled to the frame via the commissure assemblies on diametrically opposite sides of the frame from each other.

Example 125—The prosthetic heart valve of any example herein, particularly any one of examples 112-123, wherein the valvular structure is a tricuspid structure with three leaflets and three commissure assemblies, and the valvular structure is coupled to the frame via the three commissure assemblies equally spaced along a circumferential direction of the frame.

Example 126—The prosthetic heart valve of any example herein, particularly any one of examples 112-125, wherein the frame is formed of a plastically-expandable material or a self-expanding material.

Example 127—The prosthetic heart valve of any example herein, particularly any one of examples 112-126, wherein the prosthetic heart valve is constructed for implantation in an existing heart valve or vasculature within a patient that experiences a pressure gradient of 30 mmHg or less.

Example 128—The prosthetic heart valve of any example herein, particularly any one of examples 112-127, wherein the prosthetic heart valve is constructed for implantation at a mitral position or a tricuspid position.

Example 129—An assembly comprises a delivery apparatus comprising an elongated shaft and the prosthetic heart valve of any one of examples 112-128 mounted on the elongated shaft in the radially-compressed configuration for delivery into a patient's body.

Example 130—A method of implanting a prosthetic heart valve in a patient's body comprises inserting a distal end of a delivery apparatus into vasculature of a patient, the delivery apparatus comprising an elongated shaft, the prosthetic heart valve of any one of examples 112-128 being releasably mounted in the radially-compressed configuration on the elongated shaft of the delivery apparatus; advancing the prosthetic heart valve to a desired implantation site; and using the delivery apparatus to expand the prosthetic heart valve to the radially-expanded configuration, thereby implanting the prosthetic heart valve at the desired implantation site.

Example 131—A method of implanting a prosthetic heart valve in a patient's body comprises inserting a distal end of a delivery apparatus into vasculature of a patient, the delivery apparatus comprising an elongated shaft, the prosthetic heart valve of any one of claims 112-128 being releasably mounted in the radially-compressed configuration on the elongated shaft of the delivery apparatus; advancing the prosthetic heart valve to a desired implantation site; and deploying the prosthetic heart valve from the delivery apparatus such that the prosthetic heart valve self-expands to the radially-expanded configuration, thereby implanting the prosthetic heart valve at the desired implantation site.

Example 132—The method of any example herein, particularly any one of examples 130-131, can further comprise installing a valve dock at the desired implantation site, wherein the prosthetic heart valve in the radially-expanded configuration is mounted within the valve dock.

Example 133—The method of any example herein, particularly any one of examples 130-132, wherein the advancing to the desired implantation site employs transfemoral, transventricular, transapical, or transseptal approaches.

Example 134—A method of assembling a prosthetic heart valve having a plurality of leaflets comprises providing an inner skirt over a radially-inner circumferential surface of an annular frame, the annular frame being radially collapsible and expandable between a radially-compressed configuration and a radially-expanded configuration, the annular frame having an inflow end and an outflow end separated from the inflow end along an axial direction of the frame; forming a plurality of commissure assemblies with the plurality of leaflets, each leaflet having a first portion, first and second tabs, and a second portion, the first and second tabs being on opposite sides of the first portion with respect to a centerline of the first portion, each of the tabs having a base edge and an outer edge, the outer edges of the tabs being substantially parallel to each other, the second portion of each leaflet having a half-elliptical or semi-elliptical shape that defines a cusp edge, the cusp edge of each leaflet extending from the base edge of the first tab to the base edge of the second tab, the cusp edge of each leaflet being curved along its entire length between the base edges of the first and second tabs, each commissure assembly being formed by paired tabs of adjacent leaflets; coupling each commissure assembly to the annular frame; and coupling a cusp edge portion of the second portion of each leaflet at the respective cusp edge to the inner skirt via one or more sutures, a suture line formed by the one or more sutures following a curvature of the cusp edge, the suture line being continuous from apices of the cusp edges substantially to the commissure assemblies.

Example 135—The method of any example herein, particularly example 134, wherein the providing the inner skirt comprises coupling the inner skirt to the radially-inner circumferential surface via one or more sutures.

Example 136—The method of any example herein, particularly any one of examples 134-135, further comprising, coupling an outer skirt over a radially-outer circumferential surface portion of the annular frame via one or more sutures.

Example 137—The method of any example herein, particularly any one of examples 134-136, wherein, prior to the coupling the cusp edge portion of each leaflet to the inner skirt, the second portions of adjacent leaflets are not directly coupled together.

Example 138—A prosthetic heart valve comprises an annular frame that is radially collapsible and expandable between a radially-compressed configuration and a radially-expanded configuration, the annular frame having an inflow end and an outflow end separated from the inflow end along an axial direction of the frame; and a valvular structure supported within the annular frame and comprising a plurality of leaflets. Each leaflet has a first portion; first and second tabs on opposite sides of the first portion with respect to a centerline of the first portion, each of the tabs having a base edge and an outer edge, the outer edges of the first and second tabs being substantially parallel to each other; and a second portion having a half-elliptical or semi-elliptical shape that defines a cusp edge extending from the base edge of the first tab to the base edge of the second tab, the cusp edge being curved along its entire length between the base edges of the first and second tabs. The prosthetic heart valve can further comprise an inner skirt disposed on a radially-inner circumferential surface of the annular frame and coupled thereto, the inner skirt comprising a hermetic layer constructed such that, when the prosthetic heart valve is implanted in a patient, ingrowth of cells from surrounding native tissue of the patient into the hermetic layer is prevented; and an outer skirt disposed over a radially-outer circumferential surface of the annular frame, the outer skirt covering substantially all of the radially-outer circumferential surface of the annular frame between the inflow and outflow ends. The valvular structure is coupled to the frame via a plurality of commissure assemblies formed by paired ones of the tabs from adjacent leaflets; the inner skirt is disposed between the annular frame and the second portion of each leaflet along a radial direction of the annular frame; the inner skirt extends along the axial direction of the frame from at least apices of the cusp edges of the leaflets to at least the plurality of commissure assemblies; a cusp edge portion of the second portion of each leaflet at the respective cusp edge is coupled to the inner skirt by one or more sutures, a suture line formed by the one or more sutures following a curvature of the cusp edge; and the suture line is continuous from the apices of the cusp edges substantially to the commissure assemblies.

Example 139—The prosthetic heart valve of any example herein, particularly example 138, the hermetic layer is substantially nonporous or has pores therein that are sized to discourage cellular ingrowth.

Example 140—The prosthetic heart valve of any example herein, particularly any one of examples 138-139, wherein the hermetic layer is formed of a hydrophobic polymer material comprising polytetrafluoroethylene (PTFE), expanded PTFE (ePTFE), urethane, polyurethane (PU), thermoplastic PU (TPU), silicone, or combinations or copolymers thereof.

Example 141—The prosthetic heart valve of any example herein, particularly any one of examples 138-140, wherein the inner skirt further comprises a scrim layer disposed in a region along the axial direction where the cusp edge portions attach to the inner skirt.

Example 142—The prosthetic heart valve of any example herein, particularly any one of examples 138-141, wherein the inner skirt substantially covers an entirety of the radially-inner circumferential surface of the annular frame between the inflow and outflow ends.

Example 143—The prosthetic heart valve of any example herein, particularly example 142, wherein the commissure assemblies extend radially through respective openings in the inner skirt and through respective commissure windows of the annular frame.

Example 144—The prosthetic heart valve of any example herein, particularly example 143, further comprising one or more protective covers, each protective cover comprising a second hermetic layer of hydrophobic polymer material, the second hermetic layer being substantially nonporous or having pores therein that are sized to discourage cellular ingrowth, the one or more protective covers being disposed on respective radially-outer circumferential surface portions of the annular frame where the commissure assemblies extend through the commissure windows.

Example 145—The prosthetic heart valve of any example herein, particularly example 144, wherein the one or more protective covers is a single annular cover that wraps around the radially-outer circumferential surface of the annular frame.

Example 146—The prosthetic heart valve of any example herein, particularly any one of examples 138-145, wherein the hermetic layer comprises a lamination of sublayers.

Example 147—The prosthetic heart valve of any example herein, particularly any one of examples 138-146, wherein the hermetic layer comprises a layer formed directly on the radially-inner circumferential surface of the annular frame.

Example 148—The prosthetic heart valve of any example herein, particularly any one of examples 138-147, wherein the hermetic layer comprises an electrospun, dip-coated, or spray-coated layer on the radially-inner circumferential surface of the annular frame.

Example 149—The prosthetic heart valve of any example herein, particularly any one of examples 138-148, wherein the hermetic layer is formed on the frame so as to be coupled thereto without sutures.

Example 150—The prosthetic heart valve of any example herein, particularly any one of examples 138-148, wherein the hermetic layer comprises a layer formed separate from the frame and subsequently attached to the frame.

Example 151—The prosthetic heart valve of any example herein, particularly example 150, wherein the hermetic layer is an extruded layer or cast layer attached to the annular frame by one or more sutures.

Example 152—The prosthetic heart valve of any example herein, particularly any one of examples 138-151, wherein the outer skirt is coupled to the annular frame or the inner skirt by one or more sutures.

Example 153—The prosthetic heart valve of any example herein, particularly any one of examples 138-152, where the outer skirt comprises a third hermetic layer of hydrophobic polymer material, the third hermetic layer being substantially nonporous or having pores therein that are sized to discourage cellular ingrowth.

Example 154—The prosthetic heart valve of any example herein, particularly any one of examples 138-153, wherein a portion of the outer skirt faces or overlaps a portion of the inner skirt at the inflow end of the annular frame and is coupled to said portion of the inner skirt.

Example 155—The prosthetic heart valve of any example herein, particularly example 153, wherein the inner and outer skirts are part of a same unitary skirt structure that wraps around the inflow end of the annular frame, the hermetic layer and the third hermetic layer being a same hermetic layer.

Example 156—The prosthetic heart valve of any example herein, particularly any one of examples 153-155, wherein the third hermetic layer comprises a lamination of sublayers.

Example 157—The prosthetic heart valve of any example herein, particularly any one of examples 153-156, wherein the third hermetic layer comprises a layer formed directly on the radially-outer circumferential surface of the annular frame.

Example 158—The prosthetic heart valve of any example herein, particularly any one of examples 153-157, wherein the third hermetic layer comprises an electrospun, dip-coated, or spray-coated layer on the radially-outer circumferential surface of the annular frame.

Example 159—The prosthetic heart valve of any example herein, particularly any one of examples 153-158, wherein the third hermetic layer is formed on the frame so as to be coupled thereto without sutures.

Example 160—The prosthetic heart valve of any example herein, particularly any one of examples 153-159, wherein the third hermetic layer comprises a layer formed separate from the frame and subsequently attached to the frame.

Example 161—The prosthetic heart valve of any example herein, particularly any one of examples 153-160, wherein the third hermetic layer is an extruded or cast layer attached to the annular frame by one or more sutures.

Example 162—The prosthetic heart valve of any example herein, particularly any one of examples 138-152, wherein the outer skirt comprises polyethylene terephthalate (PET).

Example 163—The prosthetic heart valve of any example herein, particularly any one of examples 138-162, wherein the suture line extends to the base edges of the first and second tabs of each leaflet, or to respective locations that are substantially adjacent to the base edges of the first and second tabs of the leaflets.

Example 164—The prosthetic heart valve of any example herein, particularly any one of examples 138-163, wherein, for each leaflet: at the base edge of the first tab, the cusp edge of the second portion has a tangent that is substantially parallel to the outer edge of the first tab, and at the base edge of the second tab, the cusp edge of the second portion has a tangent that is substantially parallel to the outer edge of the second tab.

Example 165—The prosthetic heart valve of any example herein, particularly any one of examples 138-164, wherein the outer edges of the tabs of the leaflets are substantially parallel to the axial direction of the frame.

Example 166—The prosthetic heart valve of any example herein, particularly any one of examples 138-165, wherein, in an open configuration of the valvular structure, the centerline of the first portion of each leaflet is substantially parallel to the axial direction of the frame.

Example 167—The prosthetic heart valve of any example herein, particularly any one of examples 138-166, wherein the second portions of adjacent leaflets are only indirectly coupled to each other via the coupling to the inner skirt.

Example 168—The prosthetic heart valve of any example herein, particularly any one of examples 138-167, wherein, for each leaflet, a major axis of the half-elliptical or semi-elliptical shape is substantially coincident with the base edges of the corresponding first and second tabs.

Example 169—The prosthetic heart valve of any example herein, particularly any one of examples 138-168, wherein, for each leaflet, a thickness of one or more of the first portion, the second portion, the first tab, and the second tab is less than or equal to 0.012 inches (305 μm).

Example 170—The prosthetic heart valve of any example herein, particularly any one of examples 138-169, wherein each tab of each commissure assembly comprises: a first part that extends along a circumferential direction of the frame on a radially-outer side of the respective commissure window, the first part being in contact with a coupling member; a second part that extends radially through the respective commissure window to connect the first part to the first portion of the leaflet; a third part that extends along a circumferential direction of the frame on a radially-inner side of the respective commissure window; and a fourth part that projects radially inward from the third part and contacts the first portion of the leaflet.

Example 171—The prosthetic heart valve of claim 170, wherein the coupling member comprises a flexible cloth or fabric.

Example 172—The prosthetic heart valve of any one of claims 170-171, wherein the coupling member comprises a fourth hermetic layer of hydrophobic polymer material, the fourth hermetic layer being substantially nonporous or having pores therein that are sized to discourage cellular ingrowth.

Example 173—A prosthetic heart valve comprises an annular frame that is radially collapsible and expandable between a radially-compressed configuration and a radially-expanded configuration, the annular frame having an inflow end and an outflow end separated from the inflow end along an axial direction of the frame; and a valvular structure supported within the annular frame and comprising a plurality of leaflets. Each leaflet has a first portion; first and second tabs on opposite sides of the first portion with respect to a centerline of the first portion, each of the tabs having a base edge and an outer edge, the outer edges of the first and second tabs being substantially parallel to each other; and a second portion having a half-elliptical or semi-elliptical shape that defines a cusp edge extending from the base edge of the first tab to the base edge of the second tab, the cusp edge being curved along its entire length between the base edges of the first and second tabs. The annular frame is encapsulated by a hermetic layer constructed such that, when the prosthetic heart valve is implanted in a patient, ingrowth of cells from surrounding native tissue of the patient into the hermetic layer is prevented; the valvular structure is coupled to the frame via a plurality of commissure assemblies formed by paired ones of the tabs from adjacent leaflets; a cusp edge portion of the second portion of each leaflet at the respective cusp edge is coupled to the hermetic layer by one or more sutures, a suture line formed by the one or more sutures following a curvature of the cusp edge, and the suture line is continuous from apices of the cusp edges substantially to the commissure assemblies.

Example 174—The prosthetic heart valve of any example herein, particularly example 173, wherein the hermetic layer is substantially nonporous or has pores therein that are sized to discourage cellular ingrowth.

Example 175—The prosthetic heart valve of any example herein, particularly any one of examples 173-174, wherein the hermetic layer is formed of a hydrophobic polymer material comprising polytetrafluoroethylene (PTFE), expanded PTFE (ePTFE), urethane, polyurethane (PU), thermoplastic PU (TPU), silicone, or combinations or copolymers thereof.

Example 176—The prosthetic heart valve of any example herein, particularly any one of examples 173-175, comprising a scrim layer disposed between the annular frame and the encapsulating hermetic layer, the scrim layer being in a region along the axial direction where the cusp edge portions attach to the hermetic layer.

Example 177—The prosthetic heart valve of any example herein, particularly any one of examples 173-176, wherein the hermetic layer comprises a lamination of sublayers.

Example 178—The prosthetic heart valve of any example herein, particularly any one of examples 173-177, wherein the hermetic layer comprises an electrospun, dip-coated, or spray-coated layer.

Example 179—The prosthetic heart valve of any example herein, particularly any one of examples 173-178, wherein the suture line extends to the base edges of the first and second tabs of each leaflet, or to respective locations that are substantially adjacent to the base edges of the first and second tabs of the leaflets.

Example 180—The prosthetic heart valve of any example herein, particularly any one of examples 173-179, wherein the outer edges of the tabs of the leaflets are substantially parallel to the axial direction of the frame.

Example 181—The prosthetic heart valve of any example herein, particularly any one of examples 173-180, wherein, in an open configuration of the valvular structure, the centerline of the first portion of each leaflet is substantially parallel to the axial direction of the frame.

Example 182—The prosthetic heart valve of any example herein, particularly any one of examples 173-181, further comprising an outer skirt disposed over a radially-outer circumferential surface of the encapsulated annular frame, the outer skirt covering substantially all of the radially-outer circumferential surface between the inflow and outflow ends.

Example 183—The prosthetic heart valve of any example herein, particularly example 182, wherein the outer skirt is coupled to the annular frame or to the encapsulating hermetic layer.

Example 184—The prosthetic heart valve of any example herein, particularly any one of examples 182-183, wherein the outer skirt comprises polyethylene terephthalate (PET).

Example 185—The prosthetic heart valve of any example herein, particularly any one of examples 173-184, wherein the commissure assemblies extend radially through respective openings in the encapsulating hermetic layer and through respective commissure windows of the annular frame, and the commissure assemblies are coupled to the commissure windows.

Example 186—The prosthetic heart valve of claim 185, further comprising one or more protective covers, each protective cover comprising a second hermetic layer of hydrophobic polymer material, the second hermetic layer being substantially nonporous or having pores therein that are sized to discourage cellular ingrowth, the one or more protective covers being disposed on respective radially-outer circumferential surface portions of the annular frame where the commissure assemblies extend through the commissure windows.

Example 187—The prosthetic heart valve of claim 186, wherein the one or more protective covers is a single annular cover that wraps around a portion of a radially-outer circumferential surface of the annular frame.

Example 188—The prosthetic heart valve of any example herein, particularly any one of examples 173-187, wherein each tab of each commissure assembly comprises a first part that extends along a circumferential direction of the frame on a radially-outer side of the respective commissure window, the first part being in contact with a coupling member; a second part that extends radially through the respective commissure window to connect the first part to the first portion of the leaflet; a third part that extends along a circumferential direction of the frame on a radially-inner side of the respective commissure window; and a fourth part that projects radially inward from the third part and contacts the first portion of the leaflet.

Example 189—The prosthetic heart valve of any example herein, particularly example 188, wherein the coupling member comprises a flexible cloth or fabric.

Example 190—The prosthetic heart valve of any example herein, particularly any one of examples 188-189, wherein the coupling member comprises a third hermetic layer of hydrophobic polymer material, the third hermetic layer being substantially nonporous or having pores therein that are sized to discourage cellular ingrowth.

Example 191—The prosthetic heart valve of any example herein, particularly any one of examples 173-190, wherein, for each leaflet: at the base edge of the first tab, the cusp edge of the second portion has a tangent that is substantially parallel to the outer edge of the first tab, and at the base edge of the second tab, the cusp edge of the second portion has a tangent that is substantially parallel to the outer edge of the second tab.

Example 192—The prosthetic heart valve of any example herein, particularly any one of examples 173-191, wherein the outer edges of the tabs of the leaflets are substantially parallel to the axial direction of the frame.

Example 193—The prosthetic heart valve of any example herein, particularly any one of examples 173-192, wherein, in an open configuration of the valvular structure, the centerline of the first portion of each leaflet is substantially parallel to the axial direction of the frame.

Example 194—The prosthetic heart valve of any example herein, particularly any one of examples 173-193, wherein the second portions of adjacent leaflets are only indirectly coupled to each other via the coupling to the hermetic layer.

Example 195—The prosthetic heart valve of any example herein, particularly any one of examples 173-194, wherein, for each leaflet, a major axis of the half-elliptical or semi-elliptical shape is substantially coincident with the base edges of the corresponding first and second tabs.

Example 196—The prosthetic heart valve of any example herein, particularly any one of examples 173-195, wherein, for each leaflet, a thickness of one or more of the first portion, the second portion, the first tab, and the second tab is less than or equal to 0.012 inches (305 μm).

Example 197—A prosthetic heart valve comprises an annular frame that is radially collapsible and expandable between a radially-compressed configuration and a radially-expanded configuration, the annular frame having an inflow end and an outflow end separated from the inflow end along an axial direction of the frame; valvular means for regulating blood flow through the prosthetic heart valve in a hemodynamic situation at an implanted location within a patient where a pressure gradient across the prosthetic heart valve is less than or equal to 30 mmHg; and means for preventing ingrowth of cells from surrounding native tissue of a patient onto leaflets of the valvular means.

Example 198—The prosthetic heart valve of any example herein, particularly example 197, wherein the means for preventing ingrowth of cells comprises one or more hermetic layers arranged to be disposed between the leaflets of the valvular means and surrounding native tissue when the prosthetic heart valve is implanted in the patient.

Example 199—The prosthetic heart valve of any example herein, particularly example 198, wherein each hermetic layer comprises a hydrophobic polymer material; and/or each hermetic layer is substantially nonporous or has pores therein that are sized to discourage cellular ingrowth.

Example 200—The prosthetic heart valve of any example herein, particularly example 199, wherein each hermetic layer comprises polytetrafluoroethylene (PTFE), expanded PTFE (ePTFE), urethane, polyurethane (PU), thermoplastic PU (TPU), silicone, or combinations or copolymers thereof.

Example 201—The prosthetic heart valve of any example herein, particularly any one of examples 197-200, wherein the valvular means comprises a valvular structure supported within the annular frame and comprising a plurality of leaflets, the valvular structure being coupled to the frame via a plurality of commissure assemblies formed by paired tabs of adjacent leaflets.

Example 202—The prosthetic heart valve of any example herein, particularly any one of examples 197-201, wherein the valvular means further comprises one or more sutures coupling a cusp edge portion of each leaflet of the valvular structure to an inner skirt attached to the annular frame, a suture line formed by the one or more sutures following a curvature of the cusp edge portion, the suture line being continuous from apices of the cusp edge portion substantially to the commissure assemblies.

Example 203—The prosthetic heart valve of any example herein, particularly any one of examples 197-202, wherein, in an open configuration of the valvular structure, a centerline of each leaflet is substantially parallel to the axial direction of the frame.

Example 204—The prosthetic heart valve of any example herein, particularly any one of examples 138-203, wherein the valvular structure is a bicuspid structure with two leaflets and two commissure assemblies, and the valvular structure is coupled to the frame via the commissure assemblies on diametrically opposite sides of the frame from each other.

Example 205—The prosthetic heart valve of any example herein, particularly any one of examples 138-203, wherein the valvular structure is a tricuspid structure with three leaflets and three commissure assemblies, and the valvular structure is coupled to the frame via the three commissure assemblies equally spaced along a circumferential direction of the frame.

Example 206—The prosthetic heart valve of any example herein, particularly any one of examples 138-205, wherein the frame is formed of a plastically-expandable material or a self-expanding material.

Example 207—The prosthetic heart valve of any example herein, particularly any one of examples 138-206, wherein the prosthetic heart valve is constructed for implantation in an existing heart valve at a mitral position within a patient.

Example 208—An assembly comprises a delivery apparatus comprising an elongated shaft; and the prosthetic heart valve of any one of examples 138-207 mounted on the elongated shaft in the radially-compressed configuration for delivery into a patient's body.

Example 209—A method of implanting a prosthetic heart valve in a patient's body comprises inserting a distal end of a delivery apparatus into vasculature of a patient, the delivery apparatus comprising an elongated shaft, the prosthetic heart valve of any one of examples 138-207 being releasably mounted in the radially-compressed configuration on the elongated shaft of the delivery apparatus; advancing the prosthetic heart valve to a native valve or previously-implanted prosthetic valve at a mitral position within the patient's heart; and using the delivery apparatus to expand the prosthetic heart valve to the radially-expanded configuration, thereby implanting the prosthetic heart valve at the mitral position.

Example 210—A method of implanting a prosthetic heart valve in a patient's body comprises inserting a distal end of a delivery apparatus into vasculature of a patient, the delivery apparatus comprising an elongated shaft, the prosthetic heart valve of any one of examples 138-207 being releasably mounted in the radially-compressed configuration on the elongated shaft of the delivery apparatus; advancing the prosthetic heart valve to a native valve or previously-implanted prosthetic valve at a mitral position within the patient's heart; and deploying the prosthetic heart valve from the delivery apparatus such that the prosthetic heart valve self-expands to the radially-expanded configuration, thereby implanting the prosthetic heart valve at the mitral position.

Example 211—The method of any example herein, particularly any one of examples 209-210, can further comprise installing a valve dock at the mitral position, wherein the prosthetic heart valve in the radially-expanded configuration is mounted within the valve dock.

Example 212—The method of any example herein, particularly any one of examples 209-211, wherein the advancing to the mitral position employs a transfemoral approach, a transventricular approach, a transapical approach, a transseptal approach, or any combination thereof.

Example 213—A method of assembling a prosthetic heart valve having a plurality of leaflets comprises providing an inner skirt on a radially-inner circumferential surface of an annular frame, the annular frame being radially collapsible and expandable between a radially-compressed configuration and a radially-expanded configuration, the annular frame having an inflow end and an outflow end separated from the inflow end along an axial direction of the frame, the inner skirt comprising a hermetic layer constructed such that, when the prosthetic heart valve is implanted in a patient, ingrowth of cells from surrounding native tissue of the patient into the hermetic layer is prevented, the hermetic layer comprising a layer formed directly on the radially-inner circumferential surface of the annular frame; forming a plurality of commissure assemblies with the plurality of leaflets, each leaflet having a first portion, first and second tabs, and a second portion, the first and second tabs being on opposite sides of the first portion with respect to a centerline of the first portion, each of the tabs having a base edge and an outer edge, the outer edges of the tabs being substantially parallel to each other, the second portion of each leaflet having a half-elliptical or semi-elliptical shape that defines a cusp edge, the cusp edge of each leaflet extending from the base edge of the first tab to the base edge of the second tab, the cusp edge of each leaflet being curved along its entire length between the base edges of the first and second tabs, each commissure assembly being formed by paired tabs of adjacent leaflets; coupling each commissure assembly to the annular frame, the inner skirt being disposed between the annular frame and the second portion of each leaflet along a radial direction of the annular frame, and the inner skirt extending along the axial direction of the frame from at least apices of the cusp edges of the leaflets to at least the plurality of commissure assemblies; and coupling a cusp edge portion of the second portion of each leaflet at the respective cusp edge to the inner skirt via one or more sutures, a suture line formed by the one or more sutures following a curvature of the cusp edge, the suture line being continuous from apices of the cusp edges substantially to the commissure assemblies.

Example 214—The method of any example herein, particularly example 213, wherein the providing the inner skirt comprises electrospinning, dip-coating, or spray-coating the hermetic layer onto the radially-inner circumferential surface of the annular frame.

Example 215—The method of any example herein, particularly any one of examples 213-214, wherein the hermetic layer is formed on the annular frame so as to be coupled thereto without sutures.

Example 216—The method of any example herein, particularly any one of examples 213-215, wherein the providing the inner skirt on the radially-inner circumferential surface of the annular frame comprises providing a scrim layer in a region along the axial direction where the cusp edge portions are to be coupled to the inner skirt.

Example 217—A method of assembling a prosthetic heart valve having a plurality of leaflets comprises coupling an inner skirt to a radially-inner circumferential surface of an annular frame, the annular frame being radially collapsible and expandable between a radially-compressed configuration and a radially-expanded configuration, the annular frame having an inflow end and an outflow end separated from the inflow end along an axial direction of the frame, the inner skirt comprising a hermetic layer constructed such that, when the prosthetic heart valve is implanted in a patient, ingrowth of cells from surrounding native tissue of the patient into the hermetic layer is prevented; forming a plurality of commissure assemblies with the plurality of leaflets, each leaflet having a first portion, first and second tabs, and a second portion, the first and second tabs being on opposite sides of the first portion with respect to a centerline of the first portion, each of the tabs having a base edge and an outer edge, the outer edges of the tabs being substantially parallel to each other, the second portion of each leaflet having a half-elliptical or semi-elliptical shape that defines a cusp edge, the cusp edge of each leaflet extending from the base edge of the first tab to the base edge of the second tab, the cusp edge of each leaflet being curved along its entire length between the base edges of the first and second tabs, each commissure assembly being formed by paired tabs of adjacent leaflets; coupling each commissure assembly to the annular frame, the inner skirt being disposed between the annular frame and the second portion of each leaflet along a radial direction of the annular frame, and the inner skirt extending along the axial direction of the frame from at least apices of the cusp edges of the leaflets to at least the plurality of commissure assemblies; and coupling a cusp edge portion of the second portion of each leaflet at the respective cusp edge to the inner skirt via one or more sutures, a suture line formed by the one or more sutures following a curvature of the cusp edge, the suture line being continuous from apices of the cusp edges substantially to the commissure assemblies.

Example 218—The method of any example herein, particularly example 217, wherein, prior to the coupling the inner skirt to the annular frame, the hermetic layer of the inner skirt is formed by extrusion or casting.

Example 219—The method of any example herein, particularly any one of examples 217-218, wherein the coupling the inner skirt to the annular frame is via one or more sutures.

Example 220—The method of any example herein, particularly any one of examples 217-219, wherein the coupling the inner skirt to a radially-inner circumferential surface of the annular frame comprises providing a scrim layer in a region along the axial direction where the cusp edge portions are to be coupled to the inner skirt.

Example 221—The method of any example herein, particularly any one of examples 213-220, can further comprise providing an outer skirt over a portion of a radially-outer circumferential surface of the annular frame, the outer skirt comprising a second hermetic layer of hydrophobic polymer material, the second hermetic layer being substantially nonporous or having pores therein that are sized to discourage cellular ingrowth.

Example 222: The method of any example herein, particularly example 221, wherein the providing the outer skirt comprises coupling the outer skirt to the annular frame using one or more sutures.

Example 223—The method of any example herein, particularly any one of examples 221-222, wherein the second hermetic layer of the outer skirt is formed by extruding or casting.

Example 224—The method of any example herein, particularly example 221, wherein the providing the outer skirt comprises electrospinning, dip-coating, or spray-coating the second hermetic layer onto the portion of the radially-outer circumferential surface of the annular frame.

Example 225—The method of any example herein, particularly any one of examples 213-220, further comprising providing an outer skirt over a radially-outer circumferential surface portion of the annular frame, the outer skirt comprising polyethylene terephthalate (PET).

Example 226—The method of any example herein, particularly any one of examples 213-225, wherein, prior to the coupling the cusp edge portion of each leaflet to the inner skirt, the second portions of adjacent leaflets are not directly coupled together.

Example 227—The method of any example herein, particularly any one of examples 213-226, wherein the inner skirt substantially covers an entirety of the radially-inner circumferential surface of the annular frame between the inflow and outflow ends.

Example 228—The method of any example herein, particularly example 227, further comprises forming openings in the inner skirt at locations corresponding to commissure windows of the annular frame, and wherein the coupling each commissure assembly to the annular frame comprises: inserting the tabs of the commissure assembly through one of the opening in the inner skirt and through one of the commissure windows, such that first portions of the tabs are disposed on a radially-outer side of the annular frame; separating and folding the first portions of the tabs such that the first portions extend away from each other along a circumferential direction of the annular frame; and using one or more sutures to attach the first portions of the tabs to the respective commissure window, to portions of the leaflets on a radially-inner side of the commissure window, or to any combination thereof.

Example 229—The method of any example herein, particularly example 228, can further comprise coupling one or more protective covers over the first portions of the tabs on the radially-outer side of the annular frame, each protective cover comprising a third hermetic layer of hydrophobic polymer material, the third hermetic layer being substantially nonporous or having pores therein that are sized to discourage cellular ingrowth.

Example 230—The method of any example herein, particularly example 229, wherein the one or more protective covers is a single annular cover that wraps around a radially-outer circumferential surface portion of the annular frame.

Example 231—A method of assembling a prosthetic heart valve having a plurality of leaflets comprises encapsulating an annular frame with a hermetic layer, the annular frame being radially collapsible and expandable between a radially-compressed configuration and a radially-expanded configuration, the annular frame having an inflow end and an outflow end separated from the inflow end along an axial direction of the frame, the hermetic layer being constructed such that, when the prosthetic heart valve is implanted in a patient, ingrowth of cells from surrounding native tissue of the patient into the hermetic layer is prevented; forming a plurality of commissure assemblies with the plurality of leaflets, each leaflet having a first portion, first and second tabs, and a second portion, the first and second tabs being on opposite sides of the first portion with respect to a centerline of the first portion, each of the tabs having a base edge and an outer edge, the outer edges of the tabs being substantially parallel to each other, the second portion of each leaflet having a half-elliptical or semi-elliptical shape that defines a cusp edge, the cusp edge of each leaflet extending from the base edge of the first tab to the base edge of the second tab, the cusp edge of each leaflet being curved along its entire length between the base edges of the first and second tabs, each commissure assembly being formed by paired tabs of adjacent leaflets; coupling each commissure assembly to the annular frame; and coupling a cusp edge portion of the second portion of each leaflet at the respective cusp edge to the hermetic layer via one or more sutures, a suture line formed by the one or more sutures following a curvature of the cusp edge, the suture line being continuous from apices of the cusp edges substantially to the commissure assemblies.

Example 232—The method of any example herein, particularly example 231, further comprising coupling an outer skirt over a portion of a radially-outer circumferential surface of the annular frame.

Example 233—The method of any example herein, particularly example 232, wherein the outer skirt comprises polyethylene terephthalate (PET).

Example 234—The method of any example herein, particularly any one of examples 232-233, wherein the coupling of the outer skirt to the annular frame is via one or more sutures.

Example 235—The method of any example herein, particularly any one of examples 232-234, wherein the outer skirt substantially covers an entirety of the radially-outer circumferential surface of the annular frame between the inflow and outflow ends.

Example 236—The method of any example herein, particularly any one of examples 231-235, wherein the encapsulating comprises encapsulating a scrim layer in a region along the axial direction where the cusp edge portions are to be coupled to the hermetic layer.

Example 237—The method of any example herein, particularly any one of examples 231-236, further comprises forming openings in the hermetic layer at locations corresponding to commissure windows of the annular frame, and wherein the coupling each commissure assembly to the annular frame comprises inserting the tabs of the commissure assembly through one of the opening in the hermetic layer and through one of the commissure windows, such that first portions of the tabs are disposed on a radially-outer side of the annular frame; separating and folding the first portions of the tabs such that the first portions extend away from each other along a circumferential direction of the annular frame; and using one or more sutures to attach the first portions of the tabs to the respective commissure window, to portions of the leaflets on a radially-inner side of the commissure window, to the hermetic layer, or to any combination thereof.

Example 238—The method of any example herein, particularly example 237, further comprises coupling one or more protective covers over the first portions of the tabs on the radially-outer side of the annular frame, each protective cover comprising a second hermetic layer of hydrophobic polymer material, the second hermetic layer being substantially nonporous or having pores therein that are sized to discourage cellular ingrowth.

Example 239—The method of any example herein, particularly example 238, wherein the one or more protective covers is a single annular cover that wraps around a radially-outer circumferential surface portion of the encapsulated annular frame.

Example 240—The method of any example herein, particularly any one of examples 231-239, wherein the encapsulating comprises electrospinning, dip-coating, or spray-coating the hermetic layer onto the annular frame.

Example 241—The method of any example herein, particularly any one of examples 231-240, wherein, prior to the coupling the cusp edge portion of each leaflet to the hermetic layer, the second portions of adjacent leaflets are not directly coupled together.

Example 242—The method of any example herein, particularly any one of examples 213-241, wherein each hermetic layer, some of the hermetic layers, or one of the hermetic layers comprises a lamination of sublayers.

Example 243—The method of any example herein, particularly any one of examples 213-242, wherein each hermetic layer, some of the hermetic layers, or one of the hermetic layers is substantially nonporous or has pores therein that are sized to discourage cellular ingrowth; and/or each hermetic layer, some of the hermetic layers, or one of the hermetic layers comprises a hydrophobic polymer material.

Example 244—The method of any example herein, particularly any one of examples 213-243, wherein each hermetic layer, some of the hermetic layers, or one of the hermetic layers comprises polytetrafluoroethylene (PTFE), expanded PTFE (ePTFE), urethane, polyurethane (PU), thermoplastic PU (TPU), silicone, or combinations or copolymers thereof.

Example 245—The method of any example herein, particularly any one of examples 61-103 and 209-244, wherein at least 90% of the pores in each hermetic layer, in some of the hermetic layers, or in one of the hermetic layers are less than or equal to 20 in size, 10 μm in size, 8 μm in size, or 5 μm in size.

Example 246—The method of any example herein, particularly any one of examples 61-103 and 209-245, wherein at least 90% of the pores at an inner-diameter side of each hermetic layer, of some of the hermetic layers, or of one of the hermetic layers are less than or equal to 20 μm in size, 10 μm in size, 8 μm in size, or 5 μm in size.

Example 247—The method of any example herein, particularly any of examples 61-103 and 209-246, wherein each hermetic layer, some of the hermetic layers, or one of the hermetic layers comprises a woven or knitted base material coated with or encapsulated by the hydrophobic polymer material.

Example 248—The prosthetic heart valve of any example herein, particularly any of examples 1-60 and 138-208, wherein at least 90% of the pores in each hermetic layer, in some of the hermetic layers, or in one of the hermetic layers are less than or equal to 20 μm in size, 10 μm in size, 8 μm in size, or 5 μm in size.

Example 249—The prosthetic heart valve of any example herein, particular any of examples 1-60 and 138-208, wherein at least 90% of the pores at an inner-diameter side of each hermetic layer, of some of the hermetic layers, or of one of the hermetic layers are less than or equal to 20 μm in size, 10 μm in size, 8 μm in size, or 5 μm in size

Example 250—The prosthetic heart valve of any example herein, particularly any of examples 1-60, 138-208, and 249, wherein each hermetic layer, some of the hermetic layers, or one of the hermetic layers comprises a woven or knitted base material coated with or encapsulated by the hydrophobic polymer material.

Example 251—A prosthetic heart valve comprises an annular frame comprising an inflow end, an outflow end, and a longitudinal axis extending from the inflow end to the outflow end, the annular frame movable between a radially expanded configuration and a radially compressed configuration; a valvular structure supported at least partially within the frame, the valvular structure comprising one or more leaflets that open and close to regulate blood flow through the prosthetic heart valve; and an outer skirt disposed around an outer circumferential surface of the annular frame. The outer skirt comprises a first fabric layer having a tubular shape, the first fabric layer comprising at least a first fabric section having a woven structure and at least a second fabric section having a floating structure; and a second fabric layer having a tubular shape and a woven structure, the second fabric layer disposed between the annular frame and the first fabric layer to isolate the floating structure of the first fabric layer from the annular frame.

Example 252—The prosthetic heart valve of any example herein, particularly example 251, wherein the woven structure of the first fabric section is resiliently stretchable in a circumferential direction of the first fabric layer.

Example 253—The prosthetic heart valve of any example herein, particularly example 252, wherein the woven structure of the first fabric section comprises a leno weave structure.

Example 254—The prosthetic heart valve of any example herein, particularly any of examples 251 to 253, wherein the floating structure comprises a plurality of floating yarns.

Example 255—The prosthetic heart valve of any example herein, particularly example 254, wherein the floating yarns are resiliently stretchable in a longitudinal direction of the tubular shape of the first fabric layer.

Example 256—The prosthetic heart valve of any example herein, particularly any of examples 251 to 255, wherein the woven structure of the second fabric layer comprises a plain weave structure.

Example 257—The prosthetic heart valve of any example herein, particularly any of examples 251 to 256, wherein the first fabric layer comprises a plurality of the first fabric sections and a plurality of the second fabric sections, and wherein the first fabric sections and the second fabric sections are formed as stripes extending in a circumferential direction of the tubular shape of the first fabric layer.

Example 258—The prosthetic heart valve of any example herein, particularly example 257, wherein the stripes formed by the first fabric sections alternate with the stripes formed by the second fabric sections in a longitudinal direction of the tubular shape of the first fabric layer.

Example 259—The prosthetic heart valve of any example herein, particularly any of example 257 and 258, wherein at least one of the second fabric sections is disposed between two of the first fabric sections such that the floating warp yarns of the at least one of the second fabric sections extend between and are connected to the two of the first fabric sections.

Example 260—The prosthetic heart valve of any example herein, particularly any of examples 251 to 259, wherein the first fabric layer and the second fabric layer comprise polyethylene terephthalate (PET).

Example 261—The prosthetic heart valve of any example herein, particularly any of examples 251 to 260, wherein the second fabric layer comprises a flap folded over a fabric end section of the first fabric layer.

Example 262—The prosthetic heart valve of any example herein, particularly any of examples 251 to 261, wherein the outer skirt is attached to the annular frame by one or more sutures.

Example 263—The prosthetic heart valve of any example herein, particularly any of examples 251 to 262, further comprising a hermetic layer disposed around an inner circumferential surface of the annular frame, the hermetic layer having a pore structure selected to prohibit cell ingrowth from surrounding tissue into the hermetic layer.

Example 264—A prosthetic heart valve comprises an annular frame that is radially collapsible and expandable between a radially-compressed configuration and a radially-expanded configuration, the annular frame having an inflow end and an outflow end separated from the inflow end along an axial direction of the frame; a valvular structure supported within and coupled to the annular frame, the valvular structure comprising a plurality of leaflets; and a hermetic layer having at least a portion thereof disposed on a radially inner circumferential surface of the annular frame, the hermetic layer constructed to prevent ingrowth of cells from surrounding native tissue of a patient into the hermetic layer when the prosthetic heart valve is implanted in the patient.

Example 265—The prosthetic heart valve of any example herein, particularly example 264, wherein the hermetic layer is substantially nonporous or has pores therein that are sized to discourage cellular ingrowth.

Example 266—The prosthetic heart valve of any example herein, particularly any of examples 264-265, wherein the hermetic layer is formed of a hydrophobic polymer material comprising polytetrafluoroethylene (PTFE), expanded PTFE (ePTFE), urethane, polyurethane (PU), thermoplastic PU (TPU), silicone, or combinations or copolymers thereof.

Example 267—The prosthetic heart valve of any example herein, particularly any of examples 264-266, wherein the hermetic layer comprises a lamination of sublayers.

Example 268—The prosthetic heart valve of any example herein, particularly any of examples 264-267, further comprises an inner skirt disposed on the radially inner circumferential surface of the annular frame; wherein the inner skirt comprises the hermetic layer; wherein each leaflet has tabs on opposite sides and a cusp edge portion that is curved along at least a portion thereof to form an apex at a centerline of the leaflet; wherein the valvular structure is coupled to the frame by a plurality of commissure assemblies formed by paired tabs of adjacent leaflets; wherein the inner skirt is disposed between the frame and the cusp edge portion of each leaflet along a radial direction and extends along the axial direction of the frame from at least the apices of the cusp edge portions to at least the plurality of commissure assemblies; and wherein the cusp edge portion of each leaflet is attached to the inner skirt.

Example 269—The prosthetic heart valve of any example herein, particularly example 268, wherein the inner skirt further comprises a scrim layer disposed in a region along the axial direction where the cusp edge portions attached to the inner skirt.

Example 270—The prosthetic heart valve of any example herein, particularly any of examples 268-269, further comprises one or more protective covers, each protective cover comprising a hermetic layer of hydrophobic polymer material, the hermetic layer of hydrophobic polymer material being substantially nonporous or having pores therein that are sized to discourage cellular ingrowth, the one or more protective covers being disposed on respective radially outer circumferential surface portions of the annular frame where the commissure assemblies are formed.

Example 271—The prosthetic heart valve of any example herein, particularly any of examples 264-270, further comprises an outer skirt disposed over at least a portion of a radially outer circumferential surface of the frame, the outer skirt extending along the axial direction of the frame.

Example 272—The prosthetic heart valve of any example herein, particularly example 271, wherein the outer skirt comprises a hermetic layer of hydrophobic polymer material, the hermetic layer of hydrophobic polymer material being substantially nonporous or having pores therein that are sized to discourage cellular ingrowth.

Example 273—The prosthetic heart valve of any example herein, particularly any of examples 264-267, wherein the hermetic layer encapsulates the annular frame.

Example 274—The prosthetic heart valve of any example herein, particularly any of examples 264-273, wherein each of the leaflets comprises a first portion; first and second tabs on opposite sides of the first portion with respect to a centerline of the first portion, each of the tabs having a base edge and an outer edge, the outer edges of the first and second tabs being substantially parallel to each other; and a second portion having a half-elliptical or semi-elliptical shape that defines a cusp edge, the cusp edge extending from the base edge of the first tab to the base edge of the second tab, wherein the cusp edge is curved along its entire length between the base edges of the first and second tabs.

Example 275—A method of assembling a prosthetic heart valve comprises disposing a valvular structure comprising a plurality of leaflets within an annular frame that is configured for expansion between a radially-compressed configuration and a radially-expanded configuration; coupling the valvular structure to the annular frame via a plurality of commissure assemblies formed with the plurality of leaflets; and disposing at least a portion of a hermetic layer on a radially inner circumferential surface of the annular frame, wherein the hermetic layer is constructed to prevent ingrowth of cells from surrounding native tissue of a patient into the hermetic layer when the prosthetic heart valve is implanted in the patient.

The subject matter has been described with a selection of implementations and examples, but these preferred implementations and examples are not to be taken as limiting the scope of the subject matter since many other implementations and examples are possible that fall within the scope of the subject matter. The scope of the claimed subject matter is defined by the claims.

1. A prosthetic heart valve comprising:
an annular frame that is radially collapsible and expandable between a radially-compressed configuration and a radially-expanded configuration, the annular frame having an inflow end and an outflow end separated from the inflow end along an axial direction of the annular frame;
a valvular structure supported within and coupled to the annular frame, the valvular structure comprising a plurality of leaflets; and
a hermetic layer having at least a portion thereof disposed on a radially inner circumferential surface of the annular frame, the hermetic layer constructed to prevent ingrowth of cells from surrounding native tissue of a patient into the hermetic layer when the prosthetic heart valve is implanted in the patient.

2. The prosthetic heart valve of claim 1, wherein the hermetic layer is substantially nonporous or has pores therein that are sized to discourage cellular ingrowth.

3. The prosthetic heart valve of claim 1, wherein the hermetic layer is formed of a hydrophobic polymer material comprising polytetrafluoroethylene (PTFE), expanded PTFE (ePTFE), urethane, polyurethane (PU), thermoplastic PU (TPU), silicone, or combinations or copolymers thereof.

4. The prosthetic heart valve of claim 1, wherein the hermetic layer comprises a lamination of sublayers.

5. The prosthetic heart valve of claim 1, further comprising an inner skirt disposed on the radially inner circumferential surface of the annular frame;
wherein the inner skirt comprises the hermetic layer;
wherein each leaflet has tabs on opposite sides and a cusp edge portion that is curved along at least a portion thereof to form an apex at a centerline of the leaflet;
wherein the valvular structure is coupled to the annular frame by a plurality of commissure assemblies formed by paired tabs of adjacent leaflets;
wherein the inner skirt is disposed between the annular frame and the cusp edge portion of each leaflet along a radial direction and extends along the axial direction of the annular frame from at least the apices of the cusp edge portions to at least the plurality of commissure assemblies; and
wherein the cusp edge portion of each leaflet is attached to the inner skirt.

6. The prosthetic heart valve of claim 5, wherein the inner skirt further comprises a scrim layer disposed in a region along the axial direction where the cusp edge portions are attached to the inner skirt.

7. The prosthetic heart valve of claim 5, further comprising:
one or more protective covers, each protective cover comprising a hermetic layer of hydrophobic polymer material, the hermetic layer of hydrophobic polymer material being substantially nonporous or having pores therein that are sized to discourage cellular ingrowth, the one or more protective covers being disposed on respective radially outer circumferential surface portions of the annular frame where the commissure assemblies are formed.

8. The prosthetic heart valve of claim 1, further comprising:
an outer skirt disposed over at least a portion of a radially outer circumferential surface of the annular frame, the outer skirt extending along the axial direction of the annular frame.

9. The prosthetic heart valve of claim 8, wherein the outer skirt comprises a hermetic layer of hydrophobic polymer material, the hermetic layer of hydrophobic polymer material being substantially nonporous or having pores therein that are sized to discourage cellular ingrowth.

10. The prosthetic heart valve of claim 1, wherein the hermetic layer encapsulates the annular frame.

11. The prosthetic heart valve of claim 1 wherein each of the leaflets comprises:
a first portion;
first and second tabs on opposite sides of the first portion with respect to a centerline of the first portion, each of the tabs having a base edge and an outer edge, the outer edges of the first and second tabs being substantially parallel to each other; and
a second portion having a half-elliptical or semi-elliptical shape that defines a cusp edge, the cusp edge extending from the base edge of the first tab to the base edge of the second tab, wherein the cusp edge is curved along its entire length between the base edges of the first and second tabs.

12. A method of assembling a prosthetic heart valve, the method comprising:
disposing a valvular structure comprising a plurality of leaflets within an annular frame that is configured for expansion between a radially-compressed configuration and a radially-expanded configuration;
coupling the valvular structure to the annular frame via a plurality of commissure assemblies formed with the plurality of leaflets; and
disposing at least a portion of a hermetic layer on a radially inner circumferential surface of the annular frame, wherein the hermetic layer is constructed to prevent ingrowth of cells from surrounding native tissue of a patient into the hermetic layer when the prosthetic heart valve is implanted in the patient.

13. A prosthetic heart valve comprising:
an annular frame comprising an inflow end, an outflow end, and a longitudinal axis extending from the inflow end to the outflow end, the annular frame movable between a radially expanded configuration and a radially compressed configuration;
a valvular structure supported at least partially within the frame, the valvular structure comprising one or more leaflets that open and close to regulate blood flow through the prosthetic heart valve; and
an outer skirt disposed around an outer circumferential surface of the annular frame, the outer skirt comprising:
a first fabric layer having a tubular shape, the first fabric layer comprising at least a first fabric section having a woven structure and at least a second fabric section having a floating structure; and
a second fabric layer having a tubular shape and a woven structure, the second fabric layer disposed between the annular frame and the first fabric layer to isolate the floating structure of the first fabric layer from the annular frame.

14. The prosthetic heart valve of claim 13, wherein the woven structure of the first fabric section is resiliently stretchable in a circumferential direction of the first fabric layer.

15. The prosthetic heart valve of claim 13, wherein the floating structure comprises a plurality of floating yarns, and wherein the floating yarns are resiliently stretchable in a longitudinal direction of the tubular shape of the first fabric layer.

16. The prosthetic heart valve of claim 13, wherein the woven structure of the first fabric section comprises a leno structure, and wherein the woven structure of the second fabric layer comprises a plain weave structure.

17. The prosthetic heart valve of claim 13, wherein the first fabric layer comprises a plurality of the first fabric sections and a plurality of the second fabric sections, wherein the first fabric sections and the second fabric sections are formed as stripes extending in a circumferential direction of the tubular shape of the first fabric layer.

18. The prosthetic heart valve of claim 17, wherein at least one of the second fabric sections is disposed between two of the first fabric sections such that the floating yarns of the at least one of the second fabric sections extend between and are connected to the two of the first fabric sections.

19. The prosthetic heart valve of claim 13, wherein the first fabric layer and the second fabric layer comprise polyethylene terephthalate (PET).

20. The prosthetic heart valve of claim 13, further comprising a hermetic layer disposed around an inner circumferential surface of the annular frame, the hermetic layer having a pore structure selected to prohibit cell ingrowth from surrounding tissue into the hermetic layer.
